SEQUENCE LISTING (131 sequences)

SEQ ID NO: 1 - DNA, 32 bp, synthetic construct
  misc_feature 1..32: note = Oligonucleotide: PCR-amplification of pE194
  source 1..32: mol_type = other DNA
tatatacagctggattcacaaaaaataggcac

SEQ ID NO: 2 - DNA, 33 bp, synthetic construct
  misc_feature 1..33: note = Oligonucleotide: PCR-amplification of pE194
  source 1..33: mol_type = other DNA
tatatacagctggattatgtcttttgcgcagtc

SEQ ID NO: 3 - DNA, 30 bp, synthetic construct
  misc_feature 1..30: note = Oligonucleotide: PCR-amplification of functional mRFP cassette
  source 1..30: mol_type = other DNA
tatatggatccgtaatcagggtatcgaggc

SEQ ID NO: 4 - DNA, 31 bp, synthetic construct
  misc_feature 1..31: note = Oligonucleotide: PCR-amplification of functional mRFP cassette
  source 1..31: mol_type = other DNA
tatatggatccctcattaggcgggctactaa

SEQ ID NO: 5 - DNA, 1027 bp, synthetic construct
  misc_feature 1..1027: note = homology regions: Fusion of 5-prime and 3-prime regions of aprE gene with flanking BsaI sites
  source 1..1027: mol_type = other DNA
ggtctcgacccgaagttcttttttaacatataggtaaaacaatacgaaaaaaggcgccaagtattgaagaattgcagcagccgcggcatttcccttttcgattgaagcaaaaaacgtatattgaacagtaagcattccaaaaatggaaaatactaaaatcgaacaaatatctgtttttttcttccatatctgacacacatgttgaaaaccgtttttcattgaaacatataacaagagaatgactcccgatgccagaagcctgacagagacaagcgagccggcttcaaccgctcccctttcaaatatgtactgtgcagcgcttcccgataatccccacaatgaagcccctgcaagcaccatcaatacgcctttcacatgagctgatttcatatctttcacccgtttctgtatgcgatatattgcatattttaatagatgatcgacaaggccgcaacctccttcggcaaaaaatgatctcataaaataaatgaatagtattttcataaaatgagctcaataacatattctaacaaatagcatatagaaaaagctagtgtttttagcactagctttttcttcattctgatgaaggttgttcaatattttgaatccgttccatgatcgtcggatggccgtatttaaaaatcttgacgagaaacggcgggtttgcctcgctcagcccggcttttgagagctcttgaaacgtcgaaaccgctgcatcgctgttttgcgtcagttcaatcgcatactggtcagcagctttttcctgatgcctcgaaactgcgttcgtaaatggagacgacgcgaaagagatgacccccatcagcatcagaagaagcggaagtgcggctagatcggattttcctgcaatatgaaggcttcttccatagcggccgatgatccgcttgtacagcttgtcgatcacataaaagacagcaagggataaaagcagatacccgccaagtcctatgtaaacatgcttcatcacatagtgccccatttcgtgcgccatgatgctcaggagacc

SEQ ID NO: 6 - DNA, 21 bp, synthetic construct
  misc_feature 1..21: note = Oligonucleotide: PCR-amplification of aprE genomic region
  source 1..21: mol_type = other DNA
ccggttgtcattgatccttta

SEQ ID NO: 7 - DNA, 21 bp, synthetic construct
  misc_feature 1..21: note = Oligonucleotide: PCR-amplification of aprE genomic region
  source 1..21: mol_type = other DNA
atcctcctgcaaaaaccgtat

SEQ ID NO: 8 - DNA, 1022 bp, synthetic construct
  misc_feature 1..1022: note = homology regions: Fusion of 5-prime and 3-prime regions of amyB gene with flanking BsaI sites
  source 1..1022: mol_type = other DNA
ggtctcgacccacaaggctgtcaaacgaaaaagcgtatcaggagattaacgacacgcaagaaatgatcgaaaaaatcagcggacacctgcctgtacacttgcgtcctccatacggcgggatcaatgattccgtccgctcgctttccaatctgaaggtttcattgtgggatgttgatccggaagattggaagtacaaaaataagcaaaagattgtcaatcatgtcatgagccatgcgggagacggaaaaatcgtcttaatgcacgatatttatgcaacgtccgcagatgctgctgaagagattattaaaaagctgaaagcaaaaggctatcaattggtaactgtatctcagcttgaagaagtgaagaagcagagaggctattgaataaatgagtagaaagcgccatatcggcgcttttcttttggaagaaaatatagggaaaatggtatttgttaaaaattcggaatatttatacaatatcatatgtttcacattgaaaggggaggagaatctagaagagcagagaggacggatttcctgaaggaaatccgtttttttattttgcccgtcttataaatttctttgattacattttataattaattttaacaaagtgtcatcagccctcaggaaggacttgctgacagtttgaatcgcataggtaaggcggggatgaaatggcaacgttatctgatgtagcaaagaaagcaaatgtgtcgaaaatgacggtatcgcgggtgatcaatcatcctgagactgtgacggatgaattgaaaaagcttgttcattccgcaatgaaggagctcaattatataccgaactatgcagcaagagcgctcgttcaaaacagaacacaggtcgtcaagctgctcatactggaagaaatggatacaacagaaccttattatatgaatctgttaacgggaatcagccgcgagctggaccgtcatcattatgctttgcagcttgtcacaaggaaatctctcaatatcggccagtgcgacggcattatctcaggagacc

SEQ ID NO: 9 - DNA, 22 bp, synthetic construct
  misc_feature 1..22: note = Oligonucleotide: PCR-amplification of amyB genomic region
  source 1..22: mol_type = other DNA
agaagcatgaagggcatgcgac

SEQ ID NO: 10 - DNA, 21 bp, synthetic construct
  misc_feature 1..21: note = Oligonucleotide: PCR-amplification of amyB genomic region
  source 1..21: mol_type = other DNA
caacaacaggctgtctgacgg

SEQ ID NO: 11 - DNA, 1022 bp, synthetic construct
  misc_feature 1..1022: note = homology regions: Fusion of 5-prime and 3-prime regions of sigF gene with flanking BsaI sites
  source 1..1022: mol_type = other DNA
ggtctcgacccaaaatcattcgccttgagcaatcagagcagcgtgcacttgaaacgttgggggtggcgtcatgaaaaatgaaatgaacattcagtttacagcgctcagccaaaatgaatcgtttgcacgggtgacagtcgctgcttttatcgctcagcttgacccgacgatggatgaactgaccgaaattaaaacggtcgtatccgaagcggtcacaaacgcgatcattcacggttatgaaaactcagggcagggaaacgtatatatttccgtcactctcgaggaccatattgtctatttaacgatccgcgacgaaggagtcggcatccctgatcttgaagaagcgcgccagcccctgttcacgacaaagcctgaactcgagcggtcgggaatgggctttacgatcatggaaaatttcatggatgatatttcgatcgactcctcacctgagatgggaaccacaatacacttaacaaagcacttatcaaaaagcaaagcgctttgcaattaatgaggctgctcatgttgcaggcagcctcggatgtccgatgaaaaaccggacgctcttgggagcgttccggttttttttgtgtggtaatttatggtcttttgcgcctttctgaatgataaatgggatgtacttcatactacaactataaccatcatataggaagtgacccagatggaacgtcaagtttttatcagactccgccaccggcttgaggcagatccggatgaattgattttgctcggccatatcgcacaggtagcgggtgaccgcggatacaaagaaaagcttgaacggctgcctatttatcaggtcagcaaagcggatcaaagcatggtggtgctggacgtgatgaaggtgattgaagctgtacataaatcgtttcctgaccttgatgtccaaaccgtcggcggttctgaaaccatcgtggagatccaatatccgaaaaggggtctgtcgcccgtgcttttcatcgccgtctggctgctcttgttcgtcggtgcctcaggagacc

SEQ ID NO: 12 - DNA, 19 bp, synthetic construct
  misc_feature 1..19: note = Oligonucleotide: PCR-amplification of sigF genomic region
  source 1..19: mol_type = other DNA
agcagctcggcggagaaat

SEQ ID NO: 13 - DNA, 21 bp, synthetic construct
  misc_feature 1..21: note = Oligonucleotide: PCR-amplification of sigF genomic region
  source 1..21: mol_type = other DNA
accttgcccgtcaccatttcg

SEQ ID NO: 14 - DNA, 1363 bp, synthetic construct
  misc_feature 1..1363: note = homology regions: Fusion of 5-prime and 3-prime regions of pga genes with flanking BsaI sites
  source 1..1363: mol_type = other DNA
ggtctcgacccgaacactgaaattttagaccgggctgggatatcgagacatatataggggcgtttaatggcgaatacagtaacaatgagaatagtaagaaaaattaaaaatgttaaagtttgatgaattatcattgaaaaaaattaatggctttttaaatcctaggattttaacctaaaatctgaagaaataaggtggatcgaacgactcacaaaatatttggatttgtcaatgaatcccgctttatgctaaaagagattttcattttttgatagatggtctgattgtcataggacggatttgttttgaagagggaacattggtgactttttaacctgttcgaaaagagcgaaaatactaaaagaaaagagacatcccggctgacagcccatttaaaggggattgcggccgggggaaaaaagagatcctgaatccatccttcaacctttcatctgaaatagggagaaaagtacaaaaatcataatgtcgaattttgaaagcgcatacttaaaacgctgacaaaaatctgataggaattaagaactttcgatttccaaaaatatcaataaaaagataggcattaatgactcgggcgaggtgatctttgtcacggaaaatttcgtcgtcttctgttacataatgccgattgtgatttcatagtgaaccctgatcccggttataaaagacctgtgaaaagcggccggtttgaaagggaaacacgacaattttcttaaccggtcagtgtataaagttttatagaaaatcaggaggatatatacatggttttggggttcatgtttattgtattcttttgaagggaataaaaactgacaaatttcgactgaagcaaaatttgaaaatgcatcaccttaccaattcgggatgggaaccgcacctcatgttcatgacctctttagaatatttcccttcatctttttaatccgcgcttaggtgaaaaagctgatcatgctgtgctgagcgtttcttctcgctatgacgctgctgtacatgcaaaaaaagtcctttaaatatcccagttgaatgacgatgaaagaggaaagaagaggaggaacagatcaattgataaaaaaagcggcaaacaaaaagttggttttgttttgtggaattgcggtgctttggatgtctttatttttaacgaatcataatgatgtacgcgccgatacgatcggcgagaaaatagcggaaactgccagacagcttgagggtgcgaaatacagctacggcggagagaagccgaaaacggggtttgactcgtcaggctttgtgcaatatgtgtttcaatcgctcgatattacgcttccgagaacggtaaaggaacaatcgactcttggctcaggagacc

SEQ ID NO: 15 - DNA, 20 bp, synthetic construct
  misc_feature 1..20: note = Oligonucleotide: PCR-amplification of pga genomic region
  source 1..20: mol_type = other DNA
aaagccttctcctctctatt

SEQ ID NO: 16 - DNA, 20 bp, synthetic construct
  misc_feature 1..20: note = Oligonucleotide: PCR-amplification of pga genomic region
  source 1..20: mol_type = other DNA
ttcttgaaaaagacaaggtc

SEQ ID NO: 17 - DNA, 2421 bp, Bacillus licheniformis
  misc_feature 1..2421: note = CDS: vpr gene
  source 1..2421: mol_type = unassigned DNA
ttgagaaaaagtatcgtgcgctattttgttatggcttttattctattatttgcgttatccacattcctcaccggagtgcaggcaacttccgttcccgataaaaagtcgcctgagcttgagaaagctgaaatctacggtgatattgatgtgacgtctgataaacagacgacggttatcgtggaactgaaagaaaagtcgcttgccgaagcaaaagcggacggagaaaaacaaacgaaggcttctctaaaaacagctcgaagcaaagcgaaaagcaaagcgctgaaaacacttaaaaaagcgaaagtaaaccgcgaatacgaccgtgtattttcgggcttttctatgaaactgccggccagtgaaattccaaagctgctcgccgtcaaggaagttaaagcggtttatccaaacgcaacttacaaacctgacagcgtaaaaggaaaagacgtgacacttgcagcagacgccatttatccacagatggataaaagtgccccgttcatcggagcggatcaggcatggaaatcgggctataccggtaaaggcattaaggtagccgttattgatacgggcgtggattacacccatcccgacttaaagaataatttcggcccatataagggctatgactttgtggataatgattatgatccacaggaaacaccgacaggcgacccgcgcggcggagcgactgatcacggcactcatgttgcaggaacaatcgtcgcgaacggacagattaaaggcgtggcgcctgaagcaacgcttcttgcttaccgcgtgctcggccccggcggatcaggcacaaccgagaatgtgattgcgggcattgaaaaagccgttgcagacggagcaaaagtgatgaacctctcattgggaaattcgctgaacagccctgattatgcaacaagcatcgcactggactgggcgatggctgaaggggttgtcgccgttacgtcaaacggtaacagcggaccggaaaactggacggtcggctcgccgggaacatcaagggatgcgatttccgtcggcgcatcacagcttccgtataatgagtattcggtgacgcttccttcgtattcatcagcaaaggtgatgggataccaagaggaaaaggatcttgaagcattgaacggtcaagaggtggaactcgttgaagccggcctcggacaagctgacgatttctcaggcaaagatgtgaaaggaaaagttgcggttatccagagaggcgtcataccatttgttgataaggctgaaaatgccaaaaacgccggcgctatcggagccgtgatttacaataatgcaacaggggaaatcgaggccaacgtcatggggatggccgtaccgaccgtcaaattgtcaaaagaagagggcgaaaagcttgttcaacagatcaaagaagggaaacactccgttgtcttttctttcaaattggacaaaaagctgggggaaacaattgcctcattctcgtcccgcgggcctgtcatggatacatggatgattaaacctgacgtttcagcgccaggcgtcaacatcgtcagcaccattccgacccacgatccgaaaaacccgtacggttacggttcaaaacagggaacaagcatggcttccccgcatgtcgcgggaacggcagccatattaaagcaggccaaaccggattggacgcctgagcaaatcaaaggcgtactgatgaatacggcggaaaaactgacggatgaaaatggaaagcctctccctcacaatacgcaaggagcgggcagcatccgcatcatggaggcccttaaagcttcatccattgtaacgccgggcagccattcatacggaacattcctgaaagacaaagggaaacagacgaaaaaacaagcgttcacgattgaaaacctttcttcacacagaaaagcctatcagctcgaatactcctttaaaggaacgggcatcacggtatcaggaacggaacgagtcgtggtaccggccaatcaaacaggtaaagcagcggcaaaagtaaccgtcaattccgcgaaaacgaaagcaggcacatatgaaggcacggtttacatccgtgaagacggaagaaaagtcgccgaaatcccgctcctattgatcatcaaagagccagactacccgcgcgtcacatccgtaacagttgaaccgggagcaaagcagggcgcttacacgatcgaagcctacctgccgggcggggctgaagagctcgcatttctcgtctatgatgaaaacctgaaccttctcggccaggccggcgtttacaaaaaccagggcaaaggctatcaatcttatcaatggaacggcaaaatcaatgacgccgcatcccttaagtccggaaaatactatatgcttgcctatgcatccgccaaagggaaatcaagctacgtattgacggaagacccttttatcgtcgaataa

SEQ ID NO: 18 - DNA, 1053 bp, synthetic construct
  misc_feature 1..1053: note = homology regions: Fusion of 5-prime and 3-prime regions of vpr with flanking BsaI sites
  source 1..1053: mol_type = other DNA
ggtctcgacccggatccactagtctatctcttctcttttttccgaaaagccgcctgcctgataagcatcggcagcctgatatgtaccgccggcgaacgccgtcaccttcatctgattttctctgacaggcatcaccgcattcagcagcacggctgtccataattttaaaaatgatatcaaacctttcataccgatccctccagtttcgttttgataaaactagcaactctattaaactttcttgctctatcttatcccagcaaaatgaaaatgtttgtcacaatgtgtgtgcaaaatgattctagtttttagaagttttgttgaaaactgaaggaatcgcatgattcagcggatacaaaccatgaatgtaacttactcacagcttatcctaaggataaacacatattacccacaggatatatccacatatccacatacttattcaatatttagtataagaacgtatattccctacaatatctatacacaagtttattcacttatacacagtaaattgtgcataaatctaatgacaagccttgttgagaaccactcaacaaggcttttttatgttaaaatacggataatgcgttcaggagaagctccccttctcttcaaaacgtgaaaaaagcaatcggaggacatcgtgtatatgctttcttttatcgtattattcggcttatccttcattattgtctgctttatatttttcacgactttgtacttcgccgtcaacctgcagaagcgcgagcccaagccttttcaaaaagctgcggagcaaaccgtcgataccatcatcctcattccgctcagctggctgtttaccgctttatacatatgcattctgtttattcttttcccaatccgccattttctcgatttttttcagcaaaaacgctaaattgactgatgaaacgcttcggccagcagccggtatgaatccaatctgtcttgaaaatcgtgggtgatcgtcaccgccatgatttcgtccgttccgtaagcgccggccagttcaagcagctgttcctagctcgagccatggctcaggagacc

SEQ ID NO: 19 - DNA, 24 bp, synthetic construct
  misc_feature 1..24: note = Oligonucleotide: PCR-amplification of vpr genomic region
  source 1..24: mol_type = other DNA
ctgtcacccacttcccattatgag

SEQ ID NO: 20 - DNA, 24 bp, synthetic construct
  misc_feature 1..24: note = Oligonucleotide: PCR-amplification of vpr genomic region
  source 1..24: mol_type = other DNA
gtgaccgaaggctttccatcattg

SEQ ID NO: 21 - DNA, 4299 bp, Bacillus licheniformis
  misc_feature 1..4299: note = CDS: bpr (bacillopeptidase) gene
  source 1..4299: mol_type = unassigned DNA
ttgaaaagaaagctgagaaaaaaggcgttttccaccattttaagcggtttgttaatcggctcgctgttcatgcccgccgtttcagatgccgcggcaaagcccgcacttacttccatgaaagaacaagcagccgcagggaaggggaaaatttcaaaaactcttgtcaaacagttcaaaaaagaagatcaagtcacatttttgattaaactgaaagatcaagtcgatacaccaaaagtggccaaacaggcggaaaagaacgcgaagaaaaagtcgctgagcgctgctaaaacagaatatcaaaaacgctcggctgttgtttccgctttaagagtcaaagcggatgagtcgcaaagcgatctgaaacgttatttgaaaaagcaggaaaaacaggggaacgtaaaaaaaatcagatcctattatattgtcaacggcatggcagtccatgcgacgaaagaggtcatggaacaagtagcggcttttccggaagtagaaaaagtgcttccgaacgaaaaaaggcagctgatcaaaccgactaaacagtctgtgaaaaaatcagcagccaaggatgaaaaagaaatcgaatggaatatcaatcgagtcgacgcaccaaaagcctggaaattgggatatgacggatcgggcacggttgtcgcgtcgattgatacgggcgtgcagtgggatcatccggccttgaaagaaaaataccgcggatatgacccgaagcatcccgatcagcctaaccacgaattcagttggtatgatgcggtttcaggcgcttctgaaccatttgacgatctggagcacggcacgcatgtaaccgggacgatggtcggctcagagcctgacggccaaaaccaaatcggcgtggcgccgggtgcgaagtggattgctgtgaaagctttttcagatgacggcggaacggacgaagaccttattgcagcaggtgaatggattttggcgccaaaagataaagacggcaacccgcaccctgaaatggcgccggatgtcgtcaataactcatggtcagggggagccgggattgatgagttttacagggatatcgtaaaagcgtggcgagctgccggcatctttcctgagttctctgcggggaacgtcgatttcgccaatccgggaggacctgcttcgattgccaatccggcaaactaccctgaagcgttcgcaaccggagcgacagacatcgataatcgcttagccgatttttcacttcaaggtccttccccgtatgatgagactaagcctgaaatatcggcgcctggtgtcaacattcgctcatctgtgcctggaagcggctatcaagacggctgggacggcacttcaatggctggcccgcatgtcgcagccgccgccgctcttatcaagcaagccgattcgtcgatcactgttgatgagaccgaaaagattttgatggaaactgcaacaccgctcacagacagcaaatttaccgaatcgccgaacaacggatacggccacggactcgtgaacgtgtttgacgcagtgtccgctgtgacaggcgggttgggcagagcggaaggacaagtcggaaaagaaggcgaagacaaaagtccgccggccttaaaccatcaagagatcaccgaaatttactcaggctcgaaaacgcctcttaaagttgatgtagaagacgatgtcagcatcatatcggtcaaattgagctataaaacagatgacgccgattgggagacgatcgctgccaaacaaacttccggcgactataaaaaaggaacgtacgaggtggctgtaactgtttctgaagggaaaaccctttcctacaagtggattgtcaccgatttcggaggcaacaaaacggaatccaaagtgtacgaagtcccgatttccccggctgtgacaacagggtataaacaagatttcgagaattccgccagcggctggctcacatcaggcgtgaagaattcatgggagcgcggaattcctcagtcaggcccgaacgccgcagcatcgggaaaaaacgtattcgcaacaaatctgacagggccgtatgaaagttcggctaatatgaatcttctcatgcctcctgtcagcgttccaaaaaatcaaaagctttatctcacatacaaatattggcacgatattgaagaggactttgattacggcttcgtttacgtacagcctgaaggaaaaggtgaatggatcccggctgccgagtacagcggcaagacatcagaatggaaagacggacagatcgatttgtcggaatacgggggacagacgattaaggtcatgttcaaccttcaatctgacgacagcattgaaggtgacgggctgtacatcgatgatgtagcgcttgtcaaggaagtgaagagcgccggtacgaaaaaacgattgggcgttgaaaaacagccggccaaaatgaaggataagaaaacaaagaaacgaatgattgatccgaaaaaagcaaaaccggcagaagcccttcaggaaaaaaccgaaacgaaaaaagcagctcctgcggttcttccggtgcgcgctcaggtcagcgtgctggaaacgggcaaatcgacatattccaaccaggcaacaggcgcatacagcttggctcatgcacctggaacctatacgctgaaagcggaagcgtacggatatgaatcaaacgcccaaaccgtaaaaatcgagtccgacaagacaacaacggcagactttgttttaaaagaactaaaaaaagggacgctcaccggaacgataaaaaataaaaaaacgggcgaacccgtccggcatgcaaagttgtatatcgttgaagatgcagctgtaaaaccagtccagacggatgatgacggaagctattcattaacggcctatgaaggctcgtatacggtaaaggtctctgcaaacggatattacagcagcgagttttccgttgatttaaaaggcgatgtctcgaaagatatcgatctcgatcctttcatcggctatccgggggaaataggctatgatgacggaaccggggaaaacgcctgggctttctatgaatccggcaacgggctggccgttaaaatgacgcttgaaaacgggcaggaaaaagccatgctgaaaggcggtttgtttaaattctgggatacagaattccctgatccgggcggcactgattttgcagtagaggtctatgatgcttcgggtgaaaaaggctctccgggcaaaaaaatcgccggcccgttcaaggctgaagcgctccgcacaggcgaatggacgaccgttgatttaggtgacgaaggcatcatcgtcggaaaagacttttacctcgtatacgtccagaaagaggatctggccaactcacctggacttgctacagacgaagacggagaatattccggccgcaactggcagtatacggacggctcatggtcaaaagcgccatcagaccaaggcaactttatgatccgcgctttggtcgactatgaattgtcggtgcctgtgattacgtttcctaaagacggattcatcaccaaccaaaagaacgctgtcattgaagggacatcctcgccgaatacgaccgttcatctctttaacggggatgaagaagccggaacagcggaaacggcggcagacggcactttctcaaaagaaataccgttgaataaaggggaaaatgtcatcaccgcgaaatcgtcatcggcttccggaacgactgatgcttcagaaccggtccggatcgtccttgaccagaaaaagccgaagcttacaatcgacacgccggaaagcggctcaaaactcaataaagagaccgtcaccgtgaaaggaacagtatcagacgatcacttagaatctgttcatgtaaacggcaaaaaagcagctgttgacaatggcgaatattctgcaaggattatgcttgacaacggcaaaaatgaaatcaaagtaacggcatcggatgcggcaggcaacaaaacgaccaaaaaggtcacggtagatgtcaactttgaagcgctgcaaatcaccggcttgaaaccgaccgaagatctcgagctcaaaacgggagaaaccgtgaaaatcgagtttgaaagcgccgctgatttggatgctgtctttttgatcagaatgccgctgaccaatttcaaaaccgctgcccaaaacgtaacggagctgccgatcagagaagtctcaaaaggaaaatatgaaggatattggaccgctacttcaactgcaaaagcaaaaggagcggaaatcgaggtcatcgtcagagatgattacggcaatgaaacgagacaaacggcaaaaggcaagctgtatatcaatgaaaagctgaaataa

SEQ ID NO: 22 - DNA, 1053 bp, synthetic construct
  misc_feature 1..1053: note = homology regions: Fusion of 5-prime and 3-prime regions of bpr with flanking BsaI sites
  source 1..1053: mol_type = other DNA
ggtctcgacccggatccactagtctatcaaagcttggcgggcagctgatatcttccctgcatttgcagcgggcaatgtcagcgaattcgaaccgggcggaccgggttcagttgaaaacccgtccaactatccgcaatctttcgcgattggcgcagttgacagctcaaacgcgttagccgacttttcattgcaagggccttcgccatatgacgaaatcaagcccgatatttctgcacctggcgtcagcatccgctctgcttatccgggacataagtatgctgcaatgaacgggacatcgatggcgaccccgcatgtatccggcatcgtcgccttaatgagggaagccaatccggacctcacagtcgatgaaatcgagcgcatcctgctgaaaacggcgacgcctctgactgacaaaacgtttaaaaaatccccgaacaacggctacggatacggcttggtcaacgctttaaaagcttttgacgcagcaaaaaaaactaaatgataaaaaaggacaaaacctttaagcgtaaaggtgaaaagacgctgtctttaatggcagcgtttttttcgttttacgatcgacaaattcagtacgaaaacttcaaaaaatgtacgatttacgcaacattaattgacagactttacctttgggcttgatttatacttaggaaaacaaacactaaggtcaccgagccgcagaaaggggaaggatgtggaaatctatttagatgcgatatggctgttaaacttttgttttgacttgctgcttttaatgatgaccgcatttattttaaagcgaagggttaaaaagcggaggctgatcctaggggcatttgtcgcgtcaagcatcgttctgtttatgtttacacctttttcaccgtacgtccttcatcctgccggcaaactgtcgttttcggttgtgatcgttcttgtggcatttggttttaagcggttccggttttttttgcagaatttgttttctttttattttgccacttttttaatgggaggagggattatcggagcgtagctcgagccatggctcaggagacc

SEQ ID NO: 23 - DNA, 19 bp, synthetic construct
  misc_feature 1..19: note = Oligonucleotide: PCR-amplification of bpr genomic region
  source 1..19: mol_type = other DNA
gccccgaaagatgcgaaag

SEQ ID NO: 24 - DNA, 22 bp, synthetic construct
  misc_feature 1..22: note = Oligonucleotide: PCR-amplification of bpr genomic region
  source 1..22: mol_type = other DNA
caaaacccacacagacaaacag

SEQ ID NO: 25 - DNA, 954 bp, Bacillus licheniformis
  misc_feature 1..954: note = CDS: ispA gene
  source 1..954: mol_type = unassigned DNA
atgcaaatgcgattaattccttatgtcactgacacaaagctgatggacgtgaatgaactgccggaaggaatcgatgcgattaaggcaccggagctgtggtcacaaggttttaaaggaaaagatatcactgtcgcagtgcttgatacaggctgtgacgcaacccatccagatttggcagaccgaatcatcggaggcagaaacttcactgatgatgacaacggaaaagaagatcagtttcatgattataatggacatggcacccatgtggcgggtacgattgccgcaaacgatcaaaacggaggcatttccggcgtagcacccgaagcgaacctattaatcgtcaaggtgctcggcggtgaagacggcagcggggattatgaatggatcatcaacgggatcaactacgccgttgagcaaaaagccgacattatttcaatgtcgctcggcggtcctgccgacgttccggagttgaaggaagcggtgacaaacgccgtgaagagcggagtgctcgtcgtctgcgccgcaggaaacgaaggcgacggcaatgaccgtacagaggagtactcataccctgctgcatacaacgaagtcatcgccgtcggatccgtgtcattgacgcgtgagtcttccgaattttcaaatgcgaacaaagaaattgaccttgttgcacctggagaagaaatcctctctacattgcccgaccatcaatacggaaagctgacgggaacatcgatggctacaccgcacgtcagcggcgcgctcgctctcatcaagtcagctgaagaagaggcgtttaaacggaaactgacagaacccgaactgtatgctcagttaatccgccgcacccttcctcttgattactcaaaagcgctgatcggcaacggattcttatatttgtcagcgccggaggtactggcggaaaaagccggcgaagcaaaacttctttccctttaa

SEQ ID NO: 26 - DNA, 1040 bp, synthetic construct
  misc_feature 1..1040: note = homology regions: Fusion of the 5-prime region of the ispA gene and the 3-prime end of the ispA gene with flanking BsaI sites
  source 1..1040: mol_type = other DNA
ggtctcgacccactagtttggacatttttgggcagcagcgtatctccatacaggagcctcacctaacgaagagctgtatttaccggacacaatgagattacagaaaaaaaggataactgtcaataagaaaagcaactttcatgctctctattatgtgttgattcccgttttcgtcaaaattaaaccagctctttcgcccaggtgccgagctccgttcttatttttcgctttcctgctcaaggtcgtcctttgacagcaggtgatagttttcgtgcagtaaaagcgcgtattctccttcatattttggatgcggcacaaactccgcctgctcgattgataattcgtctcttttttcgtcttcttgttctttcaccctgtatccactcctttttttaataggatatccttttgccgggataatatgttgaagcgaaatctcatatatctagtgactctctcgcccttccaccgatttcacaggtcttttcgcttacaattccagcattgttcgcaattacgtacagaggagtactcataccctgctgcatacaacgaagtcatcgccgtcggatccgtgtcattgacgcgtgagtcttccgaattttcaaatgcgaacaaagaaattgaccttgttgcacctggagaagaaatcctctctacattgcccgaccatcaatacggaaagctgacgggaacatcgatggctacaccgcacgtcagcggcgcgctcgctctcatcaagtcagctgaagaagaggcgtttaaacggaaactgacagaacccgaactgtatgctcagttaatccgccgcacccttcctcttgattactcaaaagcgctgatcggcaacggattcttatatttgtcagcgccggaggtactggcggaaaaagccggcgaagcaaaacttctttccctttaacagtctaaaggaggctgccgacaatgtcggcggcctttttcatggccatgtataaagctgaatctttttaattgcaagaattcaaaaattattttctcgagccatggctcaggagacc

SEQ ID NO: 27 - DNA, 24 bp, synthetic construct
  misc_feature 1..24: note = Oligonucleotide: PCR-amplification of ispA genomic region
  source 1..24: mol_type = other DNA
ctaccacctccttgattaatacgt

SEQ ID NO: 28 - DNA, 22 bp, synthetic construct
  misc_feature 1..22: note = Oligonucleotide: PCR-amplification of ispA genomic region
  source 1..22: mol_type = other DNA
ataacctcctcattcacagtgg

SEQ ID NO: 29 - DNA, 3123 bp, synthetic construct
  misc_feature 1..3123: note = Rreplication origin pBS72 modified
  source 1..3123: mol_type = other DNA
ggttgcggtctcaccacgcgctagtttgtctgcaactgaaaagtttataccttacctggaacaaatggttgaaacatacgaggctaatatcggcttattaggaatagtccctgtactaataaaatcaggtggatcagttgatcagtatattttggacgaagctcggaaagaatttggagatgacttgcttaattccacaattaaattaagggaaagaataaagcgatttgatgttcaaggaatcacggaagaagatactcatgataaagaagctctaaaactattcaataaccttacaatggaattgatcgaaagggtggaaggttaatggtacgaaaattaggggatctacctagaaagccacaaggcgataggtcaagcttaaagaacccttacatggatcttacagattctgaaagtaaagaaacaacagaggttaaacaaacagaaccaaaaagaaaaaaagcattgttgaaaacaatgaaagttgatgtttcaatccataataagattaaatcgctgcacgaaattctggcagcatccgaagggaattcatattacttagaggatactattgagagagctattgataagatggttgagacattacctgagagccaaaaaactttttatgaatatgaattaaaaaaaagaaccaacaaaggctgagacagactccaaacgagtctgtttttttaaaaaaaatattaggagcattgaatatatattagagaattaagaaagacatgggaataaaaatattttaaatccagtaaaaatatgataagattatttcagaatatgaagaactctgtttgtttttgatgaaaaaacaaacaaaaaaaatccacctaacggaatctcaatttaactaacagcggccaaactgagaagttaaatttgagaaggggaaaaggcggatttatacttgtatttaactatctccattttaacattttattaaaccccatacaagtgaaaatcctcttttacactgttcctttaggtgatcgcggagggacattatgagtgaagtaaacctaaaaggaaatacagatgaattagtgtattatcgacagcaaaccactggaaataaaatcgccaggaagagaatcaaaaaagggaaagaagaagtttattatgttgctgaaacggaagagaagatatggacagaagagcaaataaaaaacttttctttagacaaatttggtacgcatataccttacatagaaggtcattatacaatcttaaataattacttctttgatttttggggctattttttaggtgctgaaggaattgcgctctatgctcacctaactcgttatgcatacggcagcaaagacttttgctttcctagtctacaaacaatcgctaaaaaaatggacaagactcctgttacagttagaggctacttgaaactgcttgaaaggtacggttttatttggaaggtaaacgtccgtaataaaaccaaggataacacagaggaatccccgatttttaagattagacgtaaggttcctttgctttcagaagaacttttaaatggaaaccctaatattgaaattccagatgacgaggaagcgcatgtaaagaaggctttaaaaaaggaaaaagagggacttccaaaggttttgaaaaaagagcacgatgaatttgttaaaaaaatgatggatgagtcagaaacaattaatattccagaggctttacaatatgacacaatgtatgaagatatactcagtaaaggagaaattcgaaaagaaatcaaaaaacaaatacctaatcctacaacatcttttgagagtatatcaatgacaactgaagaggaaaaagtggacagtactttaaaaagcgaaatgcaaaatcgtgtctctaagccttcttttgatacctggtttaaaaacactaagatcaaaattgaaaataaaaattgtttattacttgtaccgagtgaatttgcatttgaatggattaagaaaagatatttagaaacaattaaaacagtccttgaagaagctggatatgttttcgaaaaaatcgaactaagaaaagtgcaataaactgctgaagtatttcagcagttttttttatttagaaatagtgaaaaaaatataatcagggaggtatcaatatttaatgagtactgatttaaatttatttagactggaattaataattaacacgtagactaattaaaatttaatgagggataaagaggatacaaaaatattaatttcaatccctattaaattttaacaagggggggattaaaatttaattagaggtttatccacaagaaaagaccctaataaaatttttactagggttataacactgattaatttcttaatgggggagggattaaaatttaatgacaaagaaaacaatcttttaagaaaagcttttaaaagataataataaaaagagctttgcgattaagcaaaactctttactttttcattgacattatcaaattcatcgatttcaaattgttgttgtatcataaagttaattctgttttgcacaaccttttcaggaatataaaacacatctgaggcttgttttataaactcagggtcgctaaagtcaatgtaacgtagcatatgatatggtatagcttccacccaagttagcctttctgcttcttctgaatgtttttcatatacttccatgggtatctctaaatgattttcctcatgtagcaaggtatgagcaaaaagtttatggaattgatagttcctctctttttcttcaacttttttatctaaaacaaacactttaacatctgagtcaatgtaagcataagatgtttttccagtcataatttcaatcccaaatcttttagacagaaattctggacgtaaatcttttggtgaaagaatttttttatgtagcaatatatccgatacagcaccttctaaaagcgttggtgaatagggcattttacctatctcctctcattttgtggaataaaaatagtcatattcgtccatctacctatcctattatcgaacagttgaactttttaatcaaggatcagtcctttttttcattattcttaaactgtgctcttaactttaacaactcgatttgtttttccagatcccgctcagagacccgttg

SEQ ID NO: 30 - DNA, 1221 bp, synthetic construct
  misc_feature 1..1221: note = functional fragment: Type-II mRFP cassette
  source 1..1221: mol_type = other DNA
ggttgcggtctcaacctttatggttttggtcggcactgccgacagcctcgcagagcacacactttatgaatataaagtatagtgtgttatactttacttggaagtggttgccggaaagagcgaaaatgcctcacatttgtgccacctaaaaaggagcgatttacatatgtcgagggtcttcggtaccttgtcggataaagctgtgttatattatgtcttggtgttaaatacacacgcttaacgatttatgcagagggtgctgcaggcggcagttctgtacaaaaatgacctaagcggaggaaaaaaaccattatattaggaggaaataacatggcctcttcagaggatgttattaaagaatttatgcggtttaaggtgaggatggaaggctcggtgaacggacatgagttcgaaattgagggagaaggtgaaggccgcccttatgaaggtactcagacagcgaaattgaaagtcacgaaaggcggaccgctgccgtttgcttgggacattctctcacctcaatttcaatatggctcaaaagcctacgtaaaacacccggctgacatccctgattacttaaagctatccttcccggagggctttaaatgggaacgagttatgaattttgaggacggcggcgtcgttactgtcacacaggattcttcccttcaggatggcgaatttatttacaaagtaaaacttcgtggaactaacttcccaagcgatggtcccgtgatgcaaaaaaaaacaatgggatgggaagcatctacggaacgtatgtatccggaggatggagccttaaagggtgaaatcaaaatgcgcctgaaacttaaagatggcggacactatgacgcggaagttaaaacaacatatatggctaaaaaaccagtccaactgccgggagcatataagacggatataaagttggacattaccagccataatgaagattacacgattgtggaacagtatgagagagcagagggcagacatagcacaggcgcgtaagaattaatgaaaaataagcggcagcctgcttttccatgcgggctgccgcttatcgggttattgcggccgcgtttataatgaagaccctagccatattctaacaaatagcatatagaaaaagctagtgtttttagcactagctttttcttcattctgatgaaggaataaaaaaagcacctgaaaaggtgtctttttttccacagagacccgttg

SEQ ID NO: 31 - DNA, 1817 bp, synthetic construct
  misc_feature 1..1817: note = functional fragment: modified kanamycin resistance gene and ColE1 replication origin
  source 1..1817: mol_type = other DNA
ggttgcggtctcagctctagttcaacaaacgggccatattgttgtataagtgatgaaatactgaatttaaaacttagtttatatgtggtaaaatgttttaatcaagtttaggaggaattaattatgaagtgtaataatggaccaataataatgactagagaagaaagaatgaagattgttcatgaaattaaggaacgaatattggataaatatggggatgatgttaaggctattggtgtttatggctctcttggtcgtcagactgatgggccctattcggatattgagatgatgtgtgtcatgtcaacagaggaagcagagttcagccatgaatggacaaccggtgagtggaaggtggaagtgaattttgatagcgaagagattctactagattatgcatctcaggtggaatcagattggccgcttacacatggtcaatttttctctattttgccgatttatgattcaggtggatacttagagaaagtgtatcaaactgctaaatcggtagaagcccaaacgttccacgatgcgatttgtgcccttatcgtagaagagctgtttgaatatgcaggcaaatggcgtaatattcgtgtgcaaggaccgacaacatttctaccatccttgactgtacaggtagcaatggcaggtgccatgttgattggtctgcatcatcgcatctgttatacgacgagcgcttcggtcttaactgaagcagttaagcaatcagatcttccttcaggttatgaccatctgtgccagttcgtaatgtctggtcaactttccgactctgagaaacttctggaatcgctagagaatttctggaatgggattcaggagtggacagaacgacacggatatatagtggatgtgtcaaaacgcataccattttgaatcgttaagggatcaactttgggagagagttcaaaattgatcctttttttataacaggaattctgagcaaaaggccagcaaaaggccaggaaccgtaaaaagcgcgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctcgccgttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgacccgtgcgccttatccggtaactatcgtcttgagtccaacccggtaagacacgacttatcgccactggcacgagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaacgagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtacaattcaaatctttttgttccattaaagggcgcgattgctgaatacctagagacccgttg

SEQ ID NO: 32 - DNA, 100 bp, synthetic construct
  misc_feature 1..100: note = Promoter: Psy flanked by BpiI restriction sites
  source 1..100: mol_type = other DNA
gaagaccttcgaatagcctattatgttcttgaaaaattgacaacaactagtaattttagtataatggactttacaaaggaggtagataatataagtcttc

SEQ ID NO: 33 - DNA, 45 bp, synthetic construct
  misc_feature 1..45: note = Oligonucleotide: Quickchange G to C, BsaI site removal
  source 1..45: mol_type = other DNA
caagacaaaaagtggaaaagtcagaccatggagagaaaagaaaat

SEQ ID NO: 34 - DNA, 45 bp, synthetic construct
  misc_feature 1..45: note = Oligonucleotide: Quickchange G to C, BsaI site removal
  source 1..45: mol_type = other DNA
attttcttttctctccatggtctgacttttccactttttgtcttg

SEQ ID NO: 35 - DNA, 47 bp, synthetic construct
  misc_feature 1..47: note = Oligonucleotide: Quickchange G to C, BpiI site removal
  source 1..47: mol_type = other DNA
aaaaggattgaaggatgcttaggtagacgagttattaatagctgaat

SEQ ID NO: 36 - DNA, 47 bp, synthetic construct
  misc_feature 1..47: note = Oligonucleotide: Quickchange G to C, BpiI site removal
  source 1..47: mol_type = other DNA
attcagctattaataactcgtctacctaagcatccttcaatcctttt

SEQ ID NO: 37 - DNA, 1015 bp, synthetic construct
  misc_feature 1..1015: note = functional fragment: Type-II mRFP cassette
  source 1..1015: mol_type = other DNA
acatatgtcgagggtcttcggtaccttgtcggataaagctgtgttatattatgtcttggtgttaaatacacacgcttaacgatttatgcagagggtgctgcaggcggcagttctgtacaaaaatgacctaagcggaggaaaaaaaccattatattaggaggaaataacatggcctcttcagaggatgttattaaagaatttatgcggtttaaggtgaggatggaaggctcggtgaacggacatgagttcgaaattgagggagaaggtgaaggccgcccttatgaaggtactcagacagcgaaattgaaagtcacgaaaggcggaccgctgccgtttgcttgggacattctctcacctcaatttcaatatggctcaaaagcctacgtaaaacacccggctgacatccctgattacttaaagctatccttcccggagggctttaaatgggaacgagttatgaattttgaggacggcggcgtcgttactgtcacacaggattcttcccttcaggatggcgaatttatttacaaagtaaaacttcgtggaactaacttcccaagcgatggtcccgtgatgcaaaaaaaaacaatgggatgggaagcatctacggaacgtatgtatccggaggatggagccttaaagggtgaaatcaaaatgcgcctgaaacttaaagatggcggacactatgacgcggaagttaaaacaacatatatggctaaaaaaccagtccaactgccgggagcatataagacggatataaagttggacattaccagccataatgaagattacacgattgtggaacagtatgagagagcagagggcagacatagcacaggcgcgtaagaattaatgaaaaataagcggcagcctgcttttccatgcgggctgccgcttatcgggttattgcggccgcgtttataatgaagaccctagccatattctaacaaatagcatatagaaaaagctagtgtttttagcactagctttttcttcattctgatgaagggagctc

SEQ ID NO: 38 - DNA, 36 bp, synthetic construct
  misc_feature 1..36: note = Oligonucleotide: PCR-amplification of functional pUK57S
  source 1..36: mol_type = other DNA
tattagaattcaaatctttttgttccattaaagggc

SEQ ID NO: 39 - DNA, 35 bp, synthetic construct
  misc_feature 1..35: note = Oligonucleotide: PCR-amplification of functional pUK57S
  source 1..35: mol_type = other DNA
tatagaattcgccactacgtaaccaacatgattaa

SEQ ID NO: 40 - DNA, 747 bp, synthetic construct
  misc_feature 1..747: note = functional fragment: GFPmut2 AF302837 with flanking BpiI restriction sites (data base AF30283799)
  source 1..747: mol_type = other DNA
attagaagacctatatgagtaaaggagaagaacttttcactggagttgtcccaattcttgttgaattagatggcgatgttaatgggcaaaaattctctgtcagtggagagggtgaaggtgatgcaacatacggaaaacttacccttaaatttatttgcactactgggaagctacctgttccatggccaacacttgtcactactttcgcgtatggtcttcaatgctttgcgagatacccagatcatatgaaacagcatgactttttcaagagtgccatgcccgaaggttatgtacaggaaagaactatattttacaaagatgacgggaactacaagacacgtgctgaagtcaagtttgaaggtgatacccttgttaatagaatcgagttaaaaggtattgattttaaagaagatggaaacattcttggacacaaaatggaatacaactataactcacataatgtatacatcatggcagacaaaccaaagaatggaatcaaagttaacttcaaaattagacacaacattaaagatggaagcgttcaattagcagaccattatcaacaaaatactccaattggcgatggccctgtccttttaccagacaaccattacctgtccacacaatctgccctttccaaagatcccaacgaaaagagagatcacatgatccttcttgagtttgtaacagctgctgggattacacatggcatggatgaactatacaaataatagcttgtcttcatta

SEQ ID NO: 41 - DNA, 37 bp, synthetic construct
  misc_feature 1..37: note = Oligonucleotide: PCR-amplification of promoter PaprE
  source 1..37: mol_type = other DNA
tatatgaagaccttcgactcgggacctctttccctcg

SEQ ID NO: 42 - DNA, 41 bp, synthetic construct
  misc_feature 1..41: note = Oligonucleotide: PCR-amplification of promoter PaprE
  source 1..41: mol_type = other DNA
tatagaagacttatatctcactctcctcctctttattcaga

SEQ ID NO: 43 - protein, 1023 aa, Bifidobacterium mongoliense
  REGION 1..1023: note = Beta Galactosidase (lactase) protein (data base WP_033513636)
MTEETRVENASEAWLTDPTIFAVNRVPAHSEHRYYAHMPQGDERSDLRQSLDGEWRVEVVQASDIDFHEEPFVAENFDDSAFERIQVPGHLQTAGLMNHKYVNIQYPWDGHENPLEPNVPESNHVALYRRKFTVSDRLAGAKAAGGTVSIVFHGMATAIYVWVNGLFAGYGEDGFTPNEFDVTDLLHDGENVVAVACYEYSSASWLEDQDFWRLHGLFRSVELAAQPHVHIDNMQIETDYDAKTGTASFDAVLSVRNAADAATISATLKDADGDVVWQTAHCVDGRTSISSGSLQDVTPWSAENPALYELDVIIIDHAGNIAEVAVQRIGFRRFRIEDGIMTINGRRIVFKGADRHEFDAKRGRAITKQDMIDDVMFCKRHNINAIRTSHYPNQDYWYELCDEYGIYLIDETNLETHGSWCLPGDVLTEETAVPGSKPQWEGACVDRVNSMMRRDYNHPSVLIWSLGNESYAGDVFRAMYRHAHDIDPNRPVHYEGVTHNRDYDDVSDIETHMYVHADAVEEYLKNDPKKPYLLCEYMHAMGNSVGNMDEYTALERYPHYQGGFIWDFIDQAILARQSDGTERLCYGGDFGDRPSDYEFSGDGLVFADRRPTPKAQEVKQLYANVHLDVTESGVVVRNDNLFTSTGGYVFALRVLADGEPVWQSDRRFDVPANETASFAIDWPVDQYRADAKELVLEVSQRLAEPTDWAPAGYELSFGQAVIDGMRPMAEAKKPIDGVVTVGRWNAGVQGSGREMLFSRTQGGMVSYAFDGREFVLRRPTITTFRALTDNDRGAGHGFERVQWLGAGRYARCVGNEIEQVDDDTLKATYVYELATPQRTRVTVAYTADTTGRVNLRVNYPGKEGELPTMPAFGIEWALPVEYSRLRFFGAGPEETYRDRKHAKLGIWSTDAFADHAPYLMPQETGNHEEVRWAEITDGHGHGLRVNRAAGADPFAMSLQPYSSFMLEEAQHQDELPVPKHMFLRVLAAQMGVGGDDSWMSPVHPQYQIPADRPISLDVDLELI

SEQ ID NO: 44 - DNA, 3072 bp, synthetic construct
  misc_feature 1..3072: note = CDS: codon optimized variant Lactase WP_033513636
  source 1..3072: mol_type = other DNA
atgacggaggaaacgcgtgttgaaaacgcctcggaagcttggctcacagatccaactatatttgccgtcaaccgagtgccggcccactcagaacaccggtattacgcccacatgccccaaggtgatgaacgttcggatctgagacagtccctggacggcgaatggcgcgtggaagttgtgcaagcatccgacatcgattttcacgaagagccgttcgttgcggaaaattttgacgattcggcttttgaaagaattcaggttccggggcatctgcagacggccggtcttatgaaccataaatatgtgaacatccagtatccttgggatggacacgaaaatccgttggaaccgaacgtgccagaatccaatcatgtcgcactgtatagaaggaaatttacagtctctgatcgtctggcgggtgcaaaggccgctgggggtaccgtctcgattgtttttcatggtatggccacagcgatctatgtgtgggtcaatggattgttcgcgggatacggtgaagatggctttactcccaatgaatttgacgtcacggacctcctgcatgacggcgaaaatgtggtagcggtcgcatgctatgagtactcctctgccagctggctcgaggaccaagatttctggcgcctgcacggattatttcggtccgtcgagctggccgctcagccccatgtgcatattgacaatatgcagattgaaacggactatgatgccaaaaccggcacggcgagctttgatgccgttttgtccgttaggaacgccgctgacgcagctacaattagcgctactcttaaagacgctgatggcgatgtagtctggcagacggctcactgtgttgatggccgcacatcaatttctagcggctcgcttcaggatgtcacaccttggtcggcggaaaacccggcactttatgaattggatgttattatcatcgatcatgccggcaacatcgcggaagtcgccgtgcagcggatcggatttcggcgttttagaatagaggacggaattatgactattaacggaaggcgcatcgtgtttaaaggagcggatcgccatgagttcgatgcgaagcgcgggcgagcaatcacaaagcaagatatgatagacgatgtgatgttttgtaaacgacataatatcaacgcaatcagaacgagtcattacccgaaccaggattactggtatgaattatgcgatgaatatggaatttacctaatcgacgaaacgaatctggaaacgcatgggagctggtgccttccgggagacgtattaaccgaagagacggcggtaccaggcagcaaaccgcaatgggaaggggcttgtgtcgatcgggtaaattcaatgatgcgccgggactataaccatccgagcgtccttatttggagtttaggaaacgagagctatgcgggagacgtgttcagagccatgtacagacacgctcacgatatcgatccgaacagaccggtacattatgaaggagtaacccacaatcgtgattatgatgacgtttcggatattgagacacatatgtatgtccacgccgacgcggtcgaagagtaccttaaaaatgatcctaaaaagccgtacctcttgtgcgagtatatgcacgcgatggggaactctgtcgggaacatggatgaatataccgctcttgaacgctacccgcattaccagggaggctttatctgggacttcatcgatcaagccatcctggcgagacaaagcgatggaacggaacgcctctgctatggcggagactttggcgatcggccgtcggactatgaatttagcggcgacggccttgttttcgcggataggcgcccgacgccgaaagcacaagaggtgaaacagctttacgcaaatgtgcatttggacgttacagaaagcggagttgtcgtccgaaatgacaacctttttacatcaacagggggctatgtctttgctttgagggtcctcgcagacggcgagccggtgtggcagagcgacagacgcttcgatgtgccggcaaatgaaacagcgtcatttgcgatcgattggcctgtagaccagtatcgcgctgatgcaaaagaacttgtgttagaagtaagccagcgtttagctgagcccacagactgggcccctgccgggtacgaactgtcatttggacaagccgttatcgacggcatgcgacctatggcagaggcaaaaaagcctattgatggggttgtcactgtaggtcgctggaatgcaggcgttcagggctctggccgcgagatgcttttcagccgcacccaaggaggcatggtttcatatgcatttgatggtcgcgaatttgtcctcagacggccgaccattacaaccttcagagcgttgacggataacgatcgcggcgctggtcatggatttgaaagggttcagtggttgggcgcaggccggtatgccaggtgcgtcggcaatgagatcgaacaagtcgatgacgacacattaaaggctacctacgtgtatgaattggcgacgcctcagcgcacgagggtcaccgtcgcctacacggcggacacgacaggacgggtgaatctccgggtcaactatccgggcaaggagggagaacttccaactatgccggcattcggtatcgagtgggcgcttcctgtcgaatattcgcgtctgcgctttttcggcgcggggccggaagagacgtatcgggaccgcaaacacgccaaactgggaatctggtccacagacgcgtttgcagatcatgcaccgtacttgatgccccaggaaacaggcaaccacgaagaggtcagatgggccgaaattacagatggacatgggcacggcttaagggtcaatcgtgcggctggagcagatccatttgcgatgtcactccaaccttactcctctttcatgctggaggaagcgcaacatcaagacgagttgcctgtccccaaacatatgttcctgagagttctggcggctcaaatgggcgtcggtggggatgactcatggatgtctccggtgcatcctcaataccaaatcccggccgaccggcctatctcattagatgtagacctggagctgatataa

SEQ ID NO: 45 - protein, 1023 aa, Bifidobacterium adolescentis
  REGION 1..1023: note = Beta Galactosidase (lactase) protein (data base WP_055309314)
MANETRIEHASETWLADSTVFEVNRVPAHSDHKCYAHDSQTNEWSDLRQSLDGEWRVEVVQASDIEFNEEPFVRENFDDSAFERIQVPGHLQMAGLMNNKYVNIQYPWDGHENPAEPNIPENNHVALYRKTFTMANRLADTKNAGGTVSIVFHGMATAIYVWVNGMFVGYGEDGFTPNEFDITEMLHDGENVVAVACYEYSSASWLEDQDFWRLHGLFRSVELAAQPHVHIENMQIESDWDPESGSASLDAALTVRNAADAATISATLKDSDGNVVWETANCADPDTSISTGSLNGIRPWSAEDPVLYEFEVTVIDHAGNIAEVAVQKVGFRRFRIEDGIMTINGKRIVFKGADRHEFDPKRGRAITEQDMIDDVVFCKRHNLNAIRTSHYPNQERWYELCDEYGIYLIDETNLETHGSWCLPGDVLTEETAVPGSKAHWEGACVDRVNSMVRRDYNHPSVLIWSLGNESYTGDVFRAMYKRVHDIDPNRPVHYEGVTHNRDYNDVTDIETRMYAHADAIEEYLKNDPQKPYISCEYMHAMGNSCGNMDEYTALERYPKYQGGFIWDFIDQAIYATQPDGTTSLRYGGDFGDRPSDYEFSGNGLVFADRKPTPKAQEVKQLYSNVHIDVAEDSVTIKNDNLFTSTGEYTFVLRVLADGEPVWQSERRFDVPAGSTEKLDVDWPLDLYRDGASELVLEVSQRLAKATNWAVAGYELAFGQTVVAGSKKASAPVKPVDGIVTVGRWNVGVQGSGREVLLSRTQGGLVSYTFNNREFVLRRPAVTTFRALTDNDRGAGHGFERAQWLGAGRYARCIGNEIEQIDENTVKASYTYELATPQRTKVTVSYTADTTGRVNLHVEYPGEPGDLPTIPAFGIEWTLPVQYSNLRFFGAGPEETYQDRKHAKLGVWSTDAFKDHAPYLMPQETGNHEDVRWAEITDEKGHGLRISRAEGAEPFAMSLQPYSSFMLEEAQHQDELPAPKHMFLRVLAEQMGVGGDDSWMSPVHPQYHIPADQPISLDVDLDLI

SEQ ID NO: 46 - DNA, 3072 bp, synthetic construct
  misc_feature 1..3072: note = CDS: codon optimized variant Lactase WP_055309314
  source 1..3072: mol_type = other DNA
atggcgaatgaaacgcggatcgaacatgcatcggaaacctggctggctgatagcacggtctttgaggtgaaccgcgttccggcacactcagatcacaaatgctacgctcacgactcacaaacgaacgaatggagtgaccttagacagtcgctcgatggcgaatggcgcgttgaggtagtccaggcgtcggacattgagtttaacgaagagccgttcgtgagggaaaatttcgacgatagcgcattcgaacgcattcaagttcctggacaccttcaaatggcgggactcatgaataacaagtacgtcaatatacagtatccttgggacggacatgaaaatcctgccgagccgaacattccggaaaacaatcacgtcgcgctttaccggaaaacttttacgatggccaacagactcgccgataccaagaacgctggtggcacggtcagcattgtgtttcatggcatggccacggccatctatgtttgggtgaatggcatgtttgtcggctacggagaagatgggtttacgcctaatgaatttgacattacagaaatgcttcatgacggtgagaacgtcgttgctgtcgcttgttatgagtattcatccgcttcgtggctagaggaccaggacttttggcgtctgcacggactgtttcgcagcgttgaacttgcagcgcaacctcacgtccatattgaaaacatgcaaatagaatccgactgggatccagagtccggcagcgcatcactggacgctgcgttgactgtacgtaatgcagccgacgcggcaaccatctccgccacgttaaaagatagtgacggaaacgtggtctgggagacagccaactgtgctgatccggacacgtccatctcaacaggatcgctgaacggaattcgcccgtggtctgcggaagatccggttctttacgaattcgaggtgacggtaattgaccacgcgggaaacatcgcagaggtcgctgtccagaaagtcggcttcagacgcttccggatcgaggatggaatcatgacgatcaatggtaagagaatcgtttttaaaggcgcagaccggcatgaattcgatccgaaacgaggccgcgcgatcacagaacaagatatgattgatgacgttgtcttttgcaaaaggcataatttgaacgcaatccggaccagccattaccctaaccaggaacggtggtacgaactttgcgacgaatatggtatttacctcatcgatgaaacgaatctggaaacccatgggtcgtggtgcctcccgggcgatgtattgaccgaagagaccgctgtccccgggtcaaaagctcattgggagggggcgtgcgtcgacagagttaattctatggttcgacgcgactataaccacccatccgtcttgatctggtctctgggaaatgaaagctatacgggcgatgttttcagagcgatgtataagcgagtgcatgatattgaccctaatcggcctgtgcattatgagggagttacacataatcgcgactacaacgatgtcaccgacattgaaacgaggatgtacgcgcacgcggatgccattgaggaatatcttaagaatgacccgcagaagccgtatattagctgcgaatacatgcacgccatgggtaattcctgcggcaatatggacgaatatacggctcttgaacggtatcctaaatatcaaggtggctttatatgggactttatagatcaggcgatttatgctacacagcccgatggtaccacttcattgcgttacgggggagattttggggatcgcccgagcgattacgagttttcaggtaatggcttggtgttcgccgatagaaaaccgacgccgaaagcccaggaagttaaacagctgtacagcaatgtacacatcgatgtggctgaggattctgttacaattaaaaatgataacttgtttacgtcgacaggggaatatacttttgtcctcagagtcttagccgatggagaacctgtttggcagtcagaacggagattcgatgttccagcgggctccacagaaaagttagatgtggattggcctttggacctgtatcgtgatggcgcgtctgaactggtcttagaagtttcccaacgtctagcgaaagcgactaattgggcggtggccggatatgaactggcatttgggcaaacggttgtcgccggcagcaagaaagcctcagcaccagtcaaaccagtggatgggatagtgacagtgggccggtggaacgtaggagtccaaggcagcgggagggaggtcctgctttcacgcacacagggcggattagtttcgtatacgtttaacaatagagaatttgttttacgcaggccggccgtaaccactttccgggccctgacagataatgataggggcgcgggccatggatttgaacgtgcccaatggctgggagcaggaagatatgcacgctgtatcgggaatgaaattgagcaaatcgatgaaaatacagtcaaggcgtcgtatacgtatgaactcgcaacacctcaaagaacaaaagtgacggtgtcttatacagcggatacaaccggcagagtcaacttacacgtagaatatccgggagaacccggcgacctcccgacgattccggcctttggcatcgaatggacgctgcccgtccagtactctaacctgaggtttttcggagcaggcccggaagagacatatcaagatcgcaaacatgccaaattaggtgtctggagcaccgacgcattcaaagaccacgcaccgtacttgatgccgcaggagacaggcaaccatgaagatgtcaggtgggcggaaatcacagacgagaaagggcatgggttgcgcatctctagggcggagggagcggaaccctttgctatgagcctgcagccgtatagttcgtttatgcttgaagaggctcaacatcaggatgaacttcctgccccgaagcatatgttcctccgtgtgttggctgaacagatgggagtaggcggagatgacagctggatgtctccggtacatccccaataccatatcccggcagaccagccgatctcattagatgtcgatcttgaccttatctaa

SEQ ID NO: 47 - protein, 30 aa, Bacillus cereus
  REGION 1..30: note = signal peptide of AmyB
MKNQFQYCCIVILSVVMLFVSLLIPQASSA

SEQ ID NO: 48 - protein, 27 aa, Bacillus subtilis
  REGION 1..27: note = signal peptide of AmyE
MFAKRFKTSLLPLFAGFLLLFHLVLAG

SEQ ID NO: 49 - protein, 29 aa, Bacillus licheniformis
  REGION 1..29: note = signal peptide of AmyL
MKQQKRLYARLLTLLFALIFLLPHSAAAA

SEQ ID NO: 50 - protein, 33 aa, Geobacillus stearothermophilus
  REGION 1..33: note = signal peptide of AmyM
MKKKTLSLFVGLMLLIGLLFSGSLPYNPNAAEA

SEQ ID NO: 51 - protein, 31 aa, Bacillus amyloliquefaciens
  REGION 1..31: note = signal peptide of AmyQ
MIQKRKRTVSFRLVLMCTLLFVSLPITKTSA

SEQ ID NO: 52 - protein, 34 aa, Geobacillus stearothermophilus
  REGION 1..34: note = signal peptide of AmyS
MLTFHRIIRKGWMFLLAFLLTALLFCPTGQPAKA

SEQ ID NO: 53 - protein, 29 aa, Bacillus subtilis
  REGION 1..29: note = signal peptide of AprE
MRSKKLWISLLFALTLIFTMAFSNMSAQA

SEQ ID NO: 54 - protein, 27 aa, Bacillus licheniformis
  REGION 1..27: note = signal peptide of AprE
MMRKKSFWLGMLTAFMLVFTMASIASA

SEQ ID NO: 55 - protein, 29 aa, Bacillus pumilus
  REGION 1..29: note = signal peptide of AprE
MKKKNVMTSVLLAVPLLFSAGFGGSMAQA

SEQ ID NO: 56 - protein, 27 aa, Bacillus clausii
  REGION 1..27: note = signal peptide of AprH
MKKPLGKIVASTALLISVAFSSSIASA

SEQ ID NO: 57 - protein, 22 aa, Bacillus subtilis
  REGION 1..22: note = signal peptide of AspB
MKLAKRVSALTPSTTLAITAKA

SEQ ID NO: 58 - protein, 29 aa, Bacillus subtilis
  REGION 1..29: note = signal peptide of BglC
MKRSISIFITCLLITLLTMGGMIASPASA

SEQ ID NO: 59 - protein, 28 aa, Bacillus subtilis
  REGION 1..28: note = signal peptide of BglS
MPYLKRVLLLLVTGLFMSLFAVTATASA

SEQ ID NO: 60 - protein, 30 aa, Bacillus subtilis
  REGION 1..30: note = signal peptide of Bpr
MRKKTKNRLISSVLSTVVISSLLFPGAAGA

SEQ ID NO: 61 - protein, 46 aa, Streptomyces sp.
  REGION 1..46: note = signal peptide of CelA
MGFGSAPIALCPLRTRRNALKRLLALLATGVSIVGLTALAGPPAQA

SEQ ID NO: 62 - protein, 49 aa, Bacillus licheniformis
  REGION 1..49: note = signal peptide of CelA
MMAEKVFSKNKIIGGKRMSYMKRSISVFIACFMVAVLGISGIIAPKASA

SEQ ID NO: 63 - protein, 35 aa, Bacillus subtilis
  REGION 1..35: note = signal peptide of Csn
MKISMQKADFWKKAAISLLVFTMFFTLMMSETVFA

SEQ ID NO: 64 - protein, 27 aa, Bacillus subtilis
  REGION 1..27: note = signal peptide of Epr
MKNMSCKLVVSVTLFFSFLTIGPLAHA

SEQ ID NO: 65 - protein, 41 aa, Bacillus licheniformis
  REGION 1..41: note = signal peptide of ForD
MKNHLYEKKKRKPLTRTIKATLAVLTMSIALVGGATVPSLA

SEQ ID NO: 66 - protein, 25 aa, Bacillus licheniformis
  REGION 1..25: note = signal peptide of GGT
MRRLAFLVVAFCLAVGCFFSPVSKA

SEQ ID NO: 67 - protein, 32 aa, Bifidobacterium bifidum
  REGION 1..32: note = signal peptide of LacZ
MAVRRLGGRIVAFAATVALSIPLGLLTNSAWA

SEQ ID NO: 68 - protein, 31 aa, Bacillus subtilis
  REGION 1..31: note = signal peptide of LipA
MKFVKRRIIALVTILMLSVTSLFALQPSAKA

SEQ ID NO: 69 - protein, 25 aa, Bacillus subtilis
  REGION 1..25: note = signal peptide of LytB
MKSCKQLIVCSLAAILLLIPSVSFA

SEQ ID NO: 70 - protein, 27 aa, Bacillus subtilis
  REGION 1..27: note = signal peptide of LytD
MKKRLIAPMLLSAASLAFFAMSGSAQA

SEQ ID NO: 71 - protein, 21 aa, Bacillus subtilis
  REGION 1..21: note = signal peptide of Pel
MKKVMLATALFLGLTPAGANA

SEQ ID NO: 72 - protein, 51 aa, Bacillus subtilis
  REGION 1..51: note = signal peptide of PhoD
MAYDSRFDEWVQKLKEESFQNNTFDRRKFIQGAGKIAGLSLGLTIAQSVGA

SEQ ID NO: 73 - protein, 19 aa, Bacillus subtilis
  REGION 1..19: note = signal peptide of PhrK
MKKLVLCVSILAVILSGVA

SEQ ID NO: 74 - protein, 31 aa, Bacillus subtilis
  REGION 1..31: note = signal peptide of Vpr
MKKGIIRFLLVSFVLFFALSTGITGVQAAPA

SEQ ID NO: 75 - protein, 32 aa, Bacillus subtilis
  REGION 1..32: note = signal peptide of WapA
MKKRKRRNFKRFIAAFLVLALMISLVPADVLA

SEQ ID NO: 76 - protein, 25 aa, Bacillus subtilis
  REGION 1..25: note = signal peptide of YbdN
MVKKWLIQFAVMLSVLSTFTYSASA

SEQ ID NO: 77 - protein, 26 aa, Bacillus subtilis
  REGION 1..26: note = signal peptide of YckD
MKRITINIITMFIAAAVISLTGTAEA

SEQ ID NO: 78 - protein, 28 aa, Bacillus subtilis
  REGION 1..28: note = signal peptide of YddT
MRKKRVITCVMAASLTLGSLLPAGYASA

SEQ ID NO: 79 - protein, 29 aa, Bacillus subtilis
  REGION 1..29: note = signal peptide of YfhK
MKKKQVMLALTAAAGLGLTALHSAPAAKA

SEQ ID NO: 80 - protein, 21 aa, Bacillus subtilis
  REGION 1..21: note = signal peptide of YfjS
MKWMCSICCAAVLLAGGAAQA

SEQ ID NO: 81 - protein, 34 aa, Bacillus subtilis
  REGION 1..34: note = signal peptide of YhfM
MKKIVAAIVVIGLVFIAFFYLYSRSGDVYQSVDA

SEQ ID NO: 82 - protein, 28 aa, Bacillus subtilis
  REGION 1..28: note = signal peptide of YjfA
MKRLFMKASLVLFAVVFVFAVKGAPAKA

SEQ ID NO: 83 - protein, 26 aa, Bacillus subtilis
  REGION 1..26: note = signal peptide of YkwD
MKKAFILSAAAAVGLFTFGGVQQASA

SEQ ID NO: 84 - protein, 42 aa, Bacillus subtilis
  REGION 1..42: note = signal peptide of YncM
MAKPLSKGGILVKKVLIAGAVGTAVLFGTLSSGIPGLPAADA

SEQ ID NO: 85 - protein, 32 aa, Bacillus subtilis
  REGION 1..32: note = signal peptide of YnfF
MIPRIKKTICVLLVCFTMLSVMLGPGATEVLA

SEQ ID NO: 86 - protein, 25 aa, Bacillus subtilis
  REGION 1..25: note = signal peptide of YobB
MKIRKILLSSALSFGMLISAVPALA

SEQ ID NO: 87 - protein, 30 aa, Bacillus subtilis
  REGION 1..30: note = signal peptide of YvcE
MRKSLITLGLASVIGTSSFLIPFTSKTASA

SEQ ID NO: 88 - protein, 26 aa, Bacillus licheniformis
  REGION 1..26: note = signal peptide of YvfO
MKNVLAVFVVLIFVLGAFGTSGPAEA

SEQ ID NO: 89 - protein, 44 aa, Bacillus subtilis
  REGION 1..44: note = signal peptide of YwbN
MSDEQKKPEQIHRRDILKWGAMAGAAVAIGASGLGGLAPLVASA

SEQ ID NO: 90 - DNA, 1140 bp, Bacillus licheniformis
  misc_feature 1..1140: note = CDS: aprE
  source 1..1140: mol_type = unassigned DNA
atgatgaggaaaaagagtttttggcttgggatgctgacggccttaatgctcgtgttcacgatggccttcagcgattccgcgtctgctgctcagccggcgaaaaatgttgaaaaggattatattgtcggatttaagtcgggagtgaaaaccgcatccgtcaaaaaggacatcatcaaagagagcggcggaaaagtggacaagcagtttagaatcatcaacgcggcaaaagcgaagctagacaaagaagcgcttgaggaagtcaaaaatgatccggatgtcgcttatgtggaagaggatcacgtagctcatgctttggcgcaaaccgttccttacggcattcctctcattaaagcggacaaagtgcaggctcaaggctacaagggagcgaacgtaaaagtcgccgtcctggatacaggaatccaagcttctcatccggacttgaacgtagtcggcggagcaagcttcgtagctggcgaagcttataacaccgacggcaacggacacggcacgcatgttgccggtacagtagctgcgcttgacaatacaacgggtgtattaggcgttgcgccgaacgtatccttgtacgcggttaaagtgctgaattcaagcggaagcggatcttacagcggcattgtaagcggaatcgagtgggcgacgacaaacggcatggatgttatcaacatgagccttggaggaccatcaggctcaacagcgatgaaacaggcggttgacaatgcatatgcaagaggggttgtcgttgtggcggctgctgggaacagcggatcttcaggaaacacgaatacaatcggctatcctgcgaaatacgactctgtcatcgcagttggcgcggtagactctaacagcaacagagcttcattttccagcgtcggagcagagcttgaagtcatggctcctggcgcaggcgtgtacagcacttacccaaccagcacttatgcaacattgaacggaacgtcaatggcttctcctcatgtagcgggagcagcagctttgatcttgtcaaaacatccgaacctttcagcttcacaagtccgcaaccgtctctccagtacggcgacttatttgggaagctccttctactatggaaaaggtctgatcaatgtcgaagctgccgctcaataa

SEQ ID NO: 91 - DNA, 1146 bp, Bacillus subtilis
  misc_feature 1..1146: note = CDS: aprE
  source 1..1146: mol_type = unassigned DNA
gtgagaagcaaaaaattgtggatcagcttgttgtttgcgttaacgttaatctttacgatggcgttcagcaacatgtctgcgcaggctgccggaaaaagcagtacagaaaagaaatacattgtcggatttaaacagacaatgagtgccatgagttccgccaagaaaaaggatgttatttctgaaaaaggcggaaaggttcaaaagcaatttaagtatgttaacgcggccgcagcaacattggatgaaaaagctgtaaaagaattgaaaaaagatccgagcgttgcatatgtggaagaagatcatattgcacatgaatatgcgcaatctgttccttatggcatttctcaaattaaagcgccggctcttcactctcaaggctacacaggctctaacgtaaaagtagctgttatcgacagcggaattgactcttctcatcctgacttaaacgtcagaggcggagcaagcttcgtaccttctgaaacaaacccataccaggacggcagttctcacggtacgcatgtagccggtacgattgccgctcttaataactcaatcggtgttctgggcgtagcgccaagcgcatcattatatgcagtaaaagtgcttgattcaacaggaagcggccaatatagctggattattaacggcattgagtgggccatttccaacaatatggatgttatcaacatgagccttggcggacctactggttctacagcgctgaaaacagtcgttgacaaagccgtttccagcggtatcgtcgttgctgccgcagccggaaacgaaggttcatccggaagcacaagcacagtcggctaccctgcaaaatatccttctactattgcagtaggtgcggtaaacagcagcaaccaaagagcttcattctccagcgcaggttctgagcttgatgtgatggctcctggcgtgtccatccaaagcacacttcctggaggcacttacggcgcttataacggaacgtccatggcgactcctcacgttgccggagcagcagcgttaattctttctaagcacccgacttggacaaacgcgcaagtccgtgatcgtttagaaagcactgcaacatatcttggaaactctttctactatggaaaagggttaatcaacgtacaagcagctgcacaataa

SEQ ID NO: 92 - DNA, 4302 bp, Bacillus subtilis
  misc_feature 1..4302: note = CDS: bpr
  source 1..4302: mol_type = unassigned DNA
atgaggaaaaaaacgaaaaacagactcatcagctctgttttaagtacagttgtcatcagttcactgctgtttccgggagcagccggggcaagcagtaaagtcacctcaccttctgttaaaaaggagcttcaatctgcggaatccattcaaaacaagatttcgagttcattaaagaaaagctttaaaaagaaagaaaaaacgacttttctgattaaatttaaagatctggctaacccagaaaaagcggcaaaagcggctgttaaaaaagcgaaatcgaagaagctgtctgccgctaagacggaatatcaaaagcgttctgctgttgtgtcatctttaaaagtcacagccgatgaatcccagcaagatgtcctaaaatacttgaacacccagaaagataaaggaaatgcagaccaaattcattcttattatgtggtgaacgggattgctgttcatgcctcaaaagaggttatggaaaaagtggtgcagtttcccgaagtggaaaaggtgcttcctaatgagaaacggcagctttttaagtcatcctccccatttaatatgaaaaaagcacagaaagctattaaagcaactgacggtgtggaatggaatgtagaccaaatcgatgccccaaaagcttgggcacttggatatgatggaactggcacggttgttgcgtccattgataccggggtggaatggaatcatccggcattaaaagagaaatatcgcggatataatccggaaaatcctaatgagcctgaaaatgaaatgaactggtatgatgccgtagcaggcgaggcaagcccttatgatgatttggctcatggaacccacgtgacaggcacgatggtgggctctgaacctgatggaacaaatcaaatcggtgtagcacctggcgcaaaatggattgctgttaaagcgttctctgaagatggcggcactgatgctgacattttggaagctggtgaatgggttttagcaccaaaggacgcggaaggaaatccccacccggaaatggctcctgatgttgtcaataactcatggggagggggctctggacttgatgaatggtacagagacatggtcaatgcctggcgtgcggccgatattttccctgagttttcagcggggaatacggatctctttattcccggcgggcctggttctatcgcaaatccggcaaactatccagaatcgtttgcaactggagcgactgatatcaataaaaagctcgctgacttttctcttcaagggccatctccatatgatgaaataaagccggaaatatctgcaccgggcgttaatattcgttcatccgttcccggtcagacatatgaggatggttgggacggcacatcaatggcagggccgcatgtatccgctgttgctgcactgctgaaacaggcgaatgcctcactttctgttgatgagatggaggatatattaaccagcacggctgaaccgctcacggattcaacatttcctgattcaccgaataacggatatggccatggtctggtgaatgcttttgatgctgtatccgctgttacagatggattagggaaagcggaaggacaagtttctgtagagggggatgaccaagagcctcctgtctatcagcatgagaaagtaactgaagcttatgaaggtggcagcctaccactgactttgacagctgaagacaatgtgagtgtgacatctgtaaagctgtcctacaagcttgatcaaggtgaatggacagaaataacggctaaacgaatcagcggtgatcatctaaaaggaacgtatcaggcagagatcccagatataaaaggaactaaactaagctataagtggatgattcacgattttggcggtcatgtcgtttcgtctgacgtatacgatgtaacagtgaaaccaagcatcacggcgggatataagcaggactttgaaactgcacccggcggctgggttgcgagcggaacaaataataactgggaatggggagttccgtcaactggcccaaatacagcagcatccggagaaaaagtatatggaacgaatttgacaggaaattatgccaactcagcaaacatgaaccttgttatgcctcctattaaagcacctgattcaggaagtctgttccttcaatttaaaagctggcacaatttagaggatgattttgattacggctacgtttttgttcttccggaaggtgaaaagaattgggagcaggctggtgtctataacggaaagacatcaagctggacggacgaagaaattgatttatcggcttataaaggccaaaacattcaagtgatgtttaaccttcaatctgatgaaagcattgcaaaagagggctggtatattgatgatgtagtgctttctgacaaatcagccggaaaaacagtcaaaaagaataagctgggcgtcgaaaagccgtctggaaagcaaaagaaaaagccagtaaatccgaaaaaggctaagccatctgcaaacacagcggtaaaacatcagaacaaggctatacagcctcaagttttgccgctcaaggcacaagtcagtgtagtggaaacaggaaaatcaacatattcagatcaatccacagggcagtacacgctgaagcacaaagcgggagactatacgcttatggcagaagcatatggttatcagtcgaaaacacaaaaagtatctttaaagacggatcagacgacacaagctaattttacgttagaagagatgaagaagggcacattaaaaggcacggtcatcaataaaacgacaggagagccggtaacaggcgcttccgtttatgttgtagaggatgctgcggtggaaccggctatgacaaacgacaaaggtgaatatatgctggaggcctatgaaggcgcttatacgattaaagtcgctgcaccgggttattacagtgatgaattttccgttgagttaaaaggtgatgttacaaaagaaactgcattgaagcctttcgtcggttatccgggtgaaattgcatatgatgacggaacagcggagaatgccaattcctattttgctgccggtaacggatgggcggtaaaaatgacgctcgctgacggcaaggacaaaggcatgcttacaggagggctgttcagattctgggatacagagttccctgatccgggcggcacagagtttaaggttgaggtatacgatgccacaggaaaagacggagcaccgggcaagaaaattgcagggccatttaacgcggaagctcttcgcaatggcgagtggactaaggtagatctcagttcaaaagggattatggtcgataaagatttttatctcgtatatatccagtcgaaacctgatccgtattcacctggattggcaatggatgaaaccggccagaattccggccgcaactggcagtatatagatggaaagtggcagccaggtgacaaagcggatggcaactatatgattcgcgcattagttgattatgaagctgctgtacctgagattacttcaccgacagacaaatcatacacaaataaggatagcgtcactgtaaaaggaaacgcttctcctggcacaacggtacacatttataatggagagaaagaagcaggagaaacgaaagctgctgcggatggcacgttccatgcaggcatcatactcaacaagggtgaaaatgagctgacggcaactgcatcaactgacaacggaacaacagatgcctccagcccaatcacggtcacgcttgatcaagaaaagcctgaattaacactggacaatccaaaggatggcgggaaaacaaataaagaaacgctgactgtcaaaggggctgtatccgatgacaatctgaaagacgtcaaggtgaatggcaaaaaagcaacagtagctgatggttcatactcagcccgtattcttttggaaaatggaagaaatgaaatcaaggtaattgctacagacttggcaggcaacaaaacgacgaaaaagacagtcattgatgtgaactttgacaagcctgtcatttccggcttaattccgggagaggataaaaacttaaaagccggtgaatctgtgaaaatcgctttctcaagcgctgaggatttagatgcaacgtttaccattcgtatgccgctgaccaatgcaagagcgagtgtgcaaaatgccaccgaactcccgttaagagaaatctctccggggagatatgaaggctattggactgccacttcttctattaaagcaaaaggagcaaaagtagaagtgatcgtccgagatgattatggaaatgaaacaagaaaaactgcgaatggaaaacttaatatgaacacagaaaattaa

SEQ ID NO: 93 - DNA, 1776 bp, Bacillus licheniformis
  misc_feature 1..1776: note = CDS: epr
  source 1..1776: mol_type = unassigned DNA
atgaaaaagttatggaaaatcgcggtttcggctgcaatgttcgtcggttttttcgcaaattcaccccgcattcaagcggaaagcaataagcaggaaaacgaggtcattgttgtatataaaaataccagcggaaaagaaaccgtcatcgaacaggcagacacggtagaacacgtttaccggcacattcccgcagccgctgtcactgcggacgacaaaacagtgcgcgaacttgaacacgatcccgacgtcctgtatgtcgaagacaacctcccggtagctgctgccgacagcaccgctctaaaagctttctccagcagcacagcgcaaaacgcctccgcgttttcacagtggaacatcaagctgattcaagccgcactggcttggaataaaggtttgaccggaaaacaggtgaagatcgcggttattgacagcgggatttccccccatgaggagctgtcgatcgccggcggtgcatcgatggtcggctataccgcttcataccgtgacgataatggccacggaacccatgttgccggaatcatcggagcgaagcataacggacgggggatcgacggcatcgcgcccggcgcgcagctgtatgccgtaaaagcgctggaccggaatggtgcgggggatctgaaaggtatcttacaaggcatcgattggtcgatccagcacggaatcgatatcatcaatatgagcctcgtcgtttcaggcgacagtcaagttctccacgatgcagtagataaagcatacaaacggggaatcattttagtcggagcgagcggaaatgccggaaacggaaaatccgtttattacccggccgcctacagcagcgtcattgccgtctcggcaaccaatgaaaaaaatcagatcgcctcattttccaacaccggcagcgccgtcgagttttccgctcccggcacatcgattatcagtacatcttccgatcgcggctatgcaatcggaagcggcacttcacaggcaacgccgcacgtcaccggcatgtttgcacttttgaaacagctgtacccaaccgcttcaaatgctgagctgcgcaagaaaatgcagttttatacaagcgatttgggcgctcccggccgtgatcatttgttcggctacggattaatccgcttcaaagaggtcacacagccattggaaaaagcgcaaaaagccgtcggacaagcggaaaagacaaagaaaaaagcggacattcaaaccgcacaaaaagcgatcgaaccgctccctgcagacgcagataaaaccgctttgaaaaagagactcaatactgttaaagagcagcttaaaaaaacagccgaaagcaaagtgaaactcgctgagaagcaaaaaaagaaaaccaatgccgactccgcccaaaaagcggtcaacgaactcgacagcggcactttcaaaacgaatttgcaaaaacggatcaacgcggtccgttcaagcctcttgaagaccgcaaagcaggccgttgccaaagctgaaaaagcggcaacagactcaaacctcggcaaagcgcaaaaagcgatcaatgagcttcccgccggaaaagacaaatcaaatctgcaaaaaaggctgaacaccgcgaaaaagcaagcagctgcagcctataataaaaaagtttctgcagcaaaggccaaagtcaaaacggccgaacaaaagagaacgaaaaaaacaaaatcagcagcgcaatcagcagtaggcaagctgaaagcatccgccgaaaaaacgaagctgcaaaaacggatcaatgccattaagctgaagtag

SEQ ID NO: 94 - DNA, 1938 bp, Bacillus subtilis
  misc_feature 1..1938: note = CDS: epr
  source 1..1938: mol_type = unassigned DNA
atgaaaaacatgtcttgcaaacttgttgtatcagtcactctgtttttcagttttctcaccataggccctctcgctcatgcgcaaaacagcagcgagaaagaggttattgtggtttataaaaacaaggccggaaaggaaaccatcctggacagtgatgctgatgttgaacagcagtataagcatcttcccgcggtagcggtcacagcagaccaggagacagtaaaagaattaaagcaggatcctgatattttgtatgtagaaaacaacgtatcatttaccgcagcagacagcacggatttcaaagtgctgtcagacggcactgacacctctgacaactttgagcaatggaaccttgagcccattcaggtgaaacaggcttggaaggcaggactgacaggaaaaaatatcaaaattgccgtcattgacagcgggatctccccccacgatgacctgtcgattgccggcgggtattcagctgtcagttatacctcttcttacaaagatgataacggccacggaacacatgtcgcagggattatcggagccaagcataacggctacggaattgacggcatcgcaccggaagcacaaatatacgcggttaaagcgcttgatcagaacggctcgggggatcttcaaagtcttctccaaggaattgactggtcgatcgcaaacaggatggacatcgtcaatatgagccttggcacgacgtcagacagcaaaatccttcatgacgccgtgaacaaagcatatgaacaaggtgttctgcttgttgccgcaagcggtaacgacggaaacggcaagccagtgaattatccggcggcatacagcagtgtcgttgcggtttcagcaacaaacgaaaagaatcagcttgcctccttttcaacaactggagatgaagttgaattttcagcaccggggacaaacatcacaagcacttacttaaaccagtattatgcaacgggaagcggaacatcccaagcgacaccgcacgccgctgccatgtttgccttgttaaaacagcgtgatcctgccgagacaaacgtccagcttcgcgaggaaatgcggaaaaacatcgttgatcttggtaccgcaggccgcgatcagcaatttggctacggcttaatccagtataaagcacaggcaacagattcagcgtacgcggcagcagagcaagcggtgaaaaaagcggaacaaacaaaagcacaaatcgatatcaacaaagcgcgagaactcatcagccagctgccgaactccgacgccaaaactgccctgcacaaaagactggataaagtacagtcatacagaaatgtaaaagatgcgaaagacaaagtcgcaaaggcagaaaaatataaaacacagcaaaccgttgacacagcacaaactgccatcaacaagctgccaaacggaacagacaaaaagaaccttcaaaaacgcttagaccaagtaaaacgatacatcgcgtcaaagcaagcgaaagacaaagttgcgaaagcggaaaaaagcaaaaagaaaacagatgtggacagcgcacaatcagcaattggcaagctgcctgcaagttcagaaaaaacgtccctgcagaaacgccttaacaaagtgaagagcaccaatttgaagacggcacagcaatccgtatctgcggctgaaaagaaatcaactgatgcaaatgcggcaaaagcacaatcagccgtcaatcagcttcaagcaggcaaggacaaaacggcattgcaaaaacggttagacaaagtgaagaaaaaggtggcggcggctgaagcaaaaaaagtggaaactgcaaaggcaaaagtgaagaaagcggaaaaagacaaaacaaagaaatcaaagacatccgctcagtctgcagtgaatcaattaaaagcatccaatgaaaaaacaaagctgcaaaaacggctgaacgccgtcaaaccgaaaaagtaa

SEQ ID NO: 95 - DNA, 951 bp, Bacillus licheniformis
  misc_feature 1..951: note = CDS: mpr
  source 1..951: mol_type = unassigned DNA
ttggttagtaaaaagagtgttaaacgaggtttgatcacaggtctcattggtatttctatttattctttaggtatgcacccggcccaagccgcgccatcgcctcatactcctgtttcaagcgatccttcatacaaagcggaaacatcggttacttatgacccaaacattaagagcgatcaatacggcttgtattcaaaagcgtttacaggcaccggcaaagtgaatgaaacaaaggaaaaagcggaaaaaaagtcacccgccaaagctccttacagcattaaatcggtgattggttctgatgatcggacaagggtcaccaacacaaccgcatatccgtacagagcgatcgttcatatttcaagcagcatcggttcatgcaccggatggatgatcggtccgaaaaccgtcgcaacagccggacactgcatctatgacacatcaagcggttcatttgccggtacagccactgtttcgccgggacggaacgggacaagctatccttacggctcagttaaatcgacgcgctactttattccgtcaggatggagaagcggaaacaccaattacgattacggcgcaatcgaactaagcgaaccgatcggcaatactgtcggatacttcggatactcgtacactacttcatcacttgttgggacaactgttaccatcagcggctacccaggcgataaaacagcaggcacacaatggcagcattcaggaccgattgccatctccgaaacgtataaattgcagtacgcaatggacacgtacggaggacaaagcggttcaccggtattcgaacaaagcagctccagaacgaactgcagcggtccgtgctcgcttgccgtacacacaaatggagtatacggcggctcctcgtacaacagaggcacccggattacaaaagaggtgttcgacaatttgaccaactggaaaaacagcgcacaataa

SEQ ID NO: 96 - DNA, 942 bp, Bacillus subtilis
  misc_feature 1..942: note = CDS: mpr
  source 1..942: mol_type = unassigned DNA
atgaaattagttccaagattcagaaaacaatggttcgcttacttaacggttttgtgtttggctttggcagcagcggtttcttttggcgtaccggcaaaagcggcagagaacccgcaaacttctgtatcgaataccggtaaagaagctgatgctacgaaaaaccaaacgtcaaaagcagatcaggtttccgccccttatgagggaaccggaaaaacaagtaaatcgttatacggcggccaaacggaactggaaaaaaacattcaaaccttacagccttcgagcattatcggaactgatgaacgcaccagaatctccagcacgacatcttttccatatagagcaaccgttcaactgtcaatcaagtatcccaacacttcaagcacttatggatgtaccggatttttagtcaatccaaatacagtcgtcacggctggacattgtgtgtacagccaggatcatggatgggcttcgacgataaccgccgcgccgggccgcaatggttcgtcatatccgtacggtacttattcaggcacgatgttttactccgtcaaaggatggacggaaagcaaagacaccaactatgattacggagctattaaattaaacggttctcctggaaacacggttggctggtacggctaccggactacaaacagcagcagtcccgtgggcctttcctcgtcagtgacaggattcccatgtgacaaaacctttggcacgatgtggtctgatacaaagccgattcgctccgctgaaacgtataagctgacctatacaaccgatacgtacggctgccaaagcggctcgcctgtttatcgaaactacagtgatacagggcagacagctattgccattcacacgaacggaggatcgtcatataacttgggaacaagggtgacgaacgatgtattcaacaatattcaatattgggcaaatcaataa

SEQ ID NO: 97 - DNA, 1617 bp, Bacillus subtilis
  misc_feature 1..1617: note = CDS: nprB
  source 1..1617: mol_type = unassigned DNA
ttgcgcaacttgaccaagacatctctattactggccggcttatgcacagcggcccaaatggtttttgtaacacatgcctcagctgaagaaagcatcgaatacgaccatacgtatcaaaccccttcatacatcatcgaaaagtcaccgcagaagccggtacaaaacacaacccagaaagaatcgctattttcctatcttgacaagcatcaaacgcagtttaagctcaaagggaatgcgaacagccattttcgcgtttcgaaaaccataaaggatccaaagacaaaacaaacgttttttaaattaacggaggtttacaaaggaattccgatttacggctttgaacaagcggtcgcgatgaaggaaaacaaacaagtgaaaagtttctttggaaaggtgcatccgcaaatcaaggacgtctccgtcacaccgtctatttctgagaaaaaagcaatacatacagcaaggcgtgagctcgaggcttccattggaaaaatcgaatatcttgatggggaaccaaaaggcgaattatatatctatccacacgacggtgaatatgatctcgcctaccttgtgagactctcgacatctgaacctgagcctggctattggcattatttcatcgatgccaaaaacggaaaggtcatcgagtcctttaatgccattcatgaagcggcaggtacaggaatcggcgtgtcaggtgatgaaaaaagctttgacgtcacagaacaaaatgggcgcttttatttggctgacgaaacaaggggaaaagggatcaatacatttgacgcgaagaacctgaacgaaaccttgtttacgcttttgtctcaactgatcgggtatacgggcaaagaaatagtcagcggcacgtccgtatttaatgaacctgcggctgtagacgcacacgcaaatgcgcaagccgtttacgattattacagcaagacatttggccgtgattcttttgatcaaaacggagcaaggattacgtctaccgtgcatgtcggcaaacaatggaacaatgctgcgtggaacggtgtccagatggtatacggggatggagacggttcgaaatttaagccgctgtctggatcgctcgacattgtcgcgcatgaaatcacacacgcagtcacacagtattccgccggtcttttatatcaaggagaacccggtgcattaaatgagtccatttctgacattatgggcgcgatggctgaccgtgatgattgggagatcggcgaagatgtctatactcctggtattgcaggagattcattgcggtcattggaggacccatctaagcagggaaatccagatcattactcgaaccgctacacaggaacagaggattatggcggagtccatatcaattcgtccattcacaataaagcagcttatcttcttgcagaaggaggcgtgcaccacggtgtacaggttgaagggattgggcgtgaagcaagtgaacaaatttactatcgggctttaacatattatgtaacggcatctacagatttcagcatgatgaagcaagcggcgattgaagctgccaatgatttatacggtgaaggctcgaagcaatcagcttcagtcgaaaaggcgtatgaggctgtcggcattctatga

SEQ ID NO: 98 - DNA, 1566 bp, Bacillus subtilis
  misc_feature 1..1566: note = CDS: nprE
  source 1..1566: mol_type = unassigned DNA
gtgggtttaggtaagaaattgtctgttgctgtcgctgcttcgtttatgagtttatcaatcagcctgccaggtgttcaggctgctgaaggtcatcagcttaaagagaatcaaacaaatttcctctccaaaaacgcgattgcgcaatcagaactctctgcaccaaatgacaaggctgtcaagcagtttttgaaaaagaacagcaacatttttaaaggtgacccttccaaaaggctgaagcttgttgaaagcacgactgatgcccttggatacaagcactttcgatatgcgcctgtcgttaacggagtgccaattaaagattcgcaagtgatcgttcacgtcgataaatccgataatgtctatgcggtcaatggtgaattacacaatcaatctgctgcaaaaacagataacagccaaaaagtctcttctgaaaaagcgctggcactcgctttcaaagctatcggcaaatcaccagacgctgtttctaacggagcggccaaaaacagcaataaagccgaattaaaagcgatagaaacaaaagacggcagctatcgtcttgcttacgacgtgacgattcgctatgtcgagcctgaacctgcaaactgggaagtcttagttgacgccgaaacaggcagcattttaaaacagcaaaataaagtagaacatgccgccgccactggaagcggaacaacgctaaagggcgcaactgttcctttgaacatctcttatgaaggcggaaaatatgttctaagagatctttcaaaaccaacaggcacccaaatcatcacatatgatttgcaaaacagacaaagccgccttccgggcacgcttgtctcaagcacaacgaaaacatttacatcttcatcacagcgggcagccgttgacgcacactataacctcggtaaagtgtacgattatttttattcaaactttaaacgaaacagctatgataacaaaggcagtaaaatcgtttcttccgttcactacggcactcaatacaataacgctgcatggacaggagaccagatgatttacggtgatggcgacggttcattcttctctccgctttccggctcattagatgtgacagcgcatgaaatgacacatggcgtcacccaagaaacagccaacttgatttatgaaaatcagccaggtgcattaaacgagtctttctctgacgtattcgggtattttaacgatacagaagactgggacatcggtgaagacattacggtcagccagcctgctcttcgcagcctgtccaaccctacaaaatacaaccagcctgacaattacgccaattaccgaaaccttccaaacacagatgaaggcgattatggcggtgtacacacaaacagcggaattccaaacaaagccgcttacaacaccatcacaaaacttggtgtatctaaatcacagcaaatctattaccgtgcgttaacaacgtacctcacgccttcttccacgttcaaagatgccaaggcagctctcattcagtctgcccgtgacctctacggctcaactgatgccgctaaagttgaagcagcctggaatgctgttggattgtaa

SEQ ID NO: 99 - DNA, 2421 bp, Bacillus subtilis
  misc_feature 1..2421: note = CDS: vpr
  source 1..2421: mol_type = unassigned DNA
ttgaaaaaggggatcattcgctttctgcttgtaagtttcgtcttattttttgcgttatccacaggcattacgggcgttcaggcagctccggcttcttcaaaaacgtcggctgatctggaaaaagccgaggtattcggtgatatcgatatgacgacaagcaaaaaaacaaccgttatagtggaattaaaagaaaaatccttggcagaagcgaaggaagcgggagaaagccaatcgaaaagcaagctgaaaaccgctcgcaccaaagcaaaaaacaaagcaatcaaagcagtgaaaaacggaaaagtaaaccgggaatatgagcaggtattctcaggcttctctatgaagcttccagctaatgagattccaaaacttctagcggtaaaagacgttaaggcagtgtacccgaacgtcacatataaaacagacaatatgaaggataaagacgtcacaatctccgaagacgccgtatctccgcaaatggatgacagtgcgccttatatcggagcaaacgatgcatgggatttaggctacacaggaaaaggcatcaaggtggcgattattgacactggggttgaatacaatcacccagatctgaagaaaaactttggacaatataaaggatacgattttgtggacaatgattacgatccaaaagaaacaccaaccggcgatccgaggggcgaggcaactgaccatggcacacacgtagccggaactgtggctgcaaacggaacgattaaaggcgtagcgcctgatgccacacttcttgcttatcgtgtgttagggcctggcggaagcggcacaacggaaaacgtcatcgcgggcgtggaacgtgcagtgcaggacggggcagatgtgatgaacctgtctctcggaaactctttaaacaacccggactgggcgacaagcacagcgcttgactgggccatgtcagaaggcgttgtcgctgttacctcaaacggcaacagcggaccgaacggctggacagtcggatcgccgggcacatcaagagaagcgatttctgtcggtgcgactcagctgccgctcaatgaatacgccgtcactttcggctcctactcttcagcaaaagtgatgggctacaacaaagaggacgacgtcaaagcgctcaataacaaagaagttgagcttgtcgaagcgggaatcggcgaagcaaaggattttgaagggaaagacctgacaggcaaagtcgccgttgtcaaacgaggcagcattgcatttgtggataaagcggataacgctaaaaaagccggtgcaatcggcatggttgtgtataacaacctctctggagaaattgaagccaatgtgccaggcatgtctgtcccaacgattaagctttcattagaagacggcgaaaaactcgtcagcgccctgaaagctggtgagacaaaaacaacattcaagttgacggtctcaaaagcgctcggtgaacaagtcgctgatttctcatcacgcggccctgttatggatacgtggatgattaagcctgatatttccgcgccaggggtcaatatcgtgagcacgatcccaacacacgatcctgaccatccatacggctacggatcaaaacaaggaacaagcatggcatcgcctcatattgccggagcggttgccgttattaaacaagccaaaccaaagtggagcgttgaacagattaaagccgccatcatgaataccgctgtcactttaaaggatagcgatggggaagtatatccgcataacgctcaaggcgcaggcagcgcaagaattatgaacgcaatcaaagccgattcgctcgtctcacctggaagctattcatacggcacgttcttgaaggaaaacggaaacgaaacaaaaaatgaaacgtttacgattgaaaatcaatcttccattagaaagtcatacacacttgaatactcatttaatggcagcggcatttccacatccggcacaagccgtgttgtgattccggcacatcaaaccgggaaagccactgcaaaagtaaaggtcaatacgaagaaaacaaaagctggcacctatgaaggaacggttatcgtcagagaaggcggaaaaacggtcgctaaggtacctacattgctgattgtgaaagagcccgattatccgagagtcacatctgtctctgtcagcgaagggtctgtacaaggtacctatcaaattgaaacctaccttcctgcgggagcggaagagctggcgttcctcgtctatgacagcaaccttgatttcgcaggccaagccggcatttataaaaaccaagataaaggttaccagtactttgactgggacggcacgattaatggcggaaccaaacttccggccggagagtattacttgctcgcatatgccgcgaacaaaggcaagtcaagccaggttttgaccgaagaacctttcactgttgaataa

SEQ ID NO: 100 - DNA, 3060 bp, Bacillus licheniformis
  misc_feature 1..3060: note = CDS: wprA
  source 1..3060: mol_type = unassigned DNA
atggcagccgaagacagcgttccgaataacgaaaccacattgaccagcgcgtcccctgttgaagcttcgtttcaaagcgatgacgaggtgcattggtataaagtcaatccttcaaatcaggaaatcgcaaactatacgcacttccgcgtcaaattgaaatcagatgcagagctgaacatttccgtctactccagcctggaaaatgcaactggtcatcaaacgtttgaccgatacaacggctactcctatgaaaataatcctgctttaatcgattttccgattgcctggaaaggtccttactacataaaagtagaaaatcaccatgatgaggaaaacgaaaccacttcaattacagatatttcttacaccatcagctatgaaggcgtcaccctgcctccgtcaatccaagaggcagaagaagagtgtccggcagaattaagcgtttccgaaagggaaacaggcaagggcatattaaaacagttaagaacgatcagggatgaagttctttcaaaaactgaaaaaggaaaagagctgtcttccctttactataaagcagctccattcatcagcgcaaaaatgctctttaacaaatcgatgagagacagtgtctacaaagacctggtgcagctgaagccgctgtttgcagatgtcgctaaaaacggacaagtgagcgcatactcgattacgaatgatgatcaaaaagcgatcagtcgcctgtacgaaacagctcgcgcgtccgttcccgagccgttgaaaaaacagctggatcaagtcgcgaaagacatcggcatcgaacaattaacaggcagcaaagtatcggctgtgcttgaaaaagcggggatggcgacagcttcatcaagcgcgcccgaaaaccgttacatcgtaaaattgaaagaaggcaaaaaaccgggatctttcaaatctaaagcccaatcatccggcgtccaggcattagagcccctcggtaaaagcaaaacggcatttaaagatatgtacgttgtggaaatgaaggaaagccgttcttccggattcaaagcggcggcaaagcaatatcaggcggcagcctccaagatcgccaagatgcctgaagtggaattcgtcgaacaggttcagcaatatgaagcactgtcaagagacacccaatatccatatcaatggtcgctcaaaaataacggcaaaaaccgtgctgcgaatgctgacatacaatttgaacagcttcagaagctgatgaaaggcaaaaagctgaaagatacagtaatcgccgtcgttgacacaggcgttgatcatacccttgcggatttaagcggcagcgtcaaaaaagacgaaggctataactatgtcggccgcacggcggatgcgatggatgacaatggccacggcacacacgtgtcaggcatcattgcagccgcgcaagacaaccatttttcgatggcgggaatcaatgcttatgccaaaattctgcctgtcaaagtgctggattcttcaggcagtggagatacggaacagattgcaaacggcatcatctatgccgccgaccacggtgcaaaagtcatcaatttaagtcttggcgggccatacagccgggtgatggaatatgcgcttaaatatgcggcttctaaaaatgtgacgatcgttgccgccaccggaaatgacggagtatcggagatttcctaccctgcatcttcgaaatatacgctttcagtcggggcgaccaataatctcgatcttgtctcggactactccaattatggaaaaggtctcgatatggtggcgccgggaaccgatattccaagcctcgttccggacgggaatgtcacttatatgagcggaacatcgatggcggcgccgcacgtggcagctgcagcaggacttcttttgtcacagaatccgtccttgaaaccaaagcaaatcgcaagcctattgaccgagacgacagcagatgtggcatttgaagagcaggataatccaaacccggattatgacctggatatagaaccggctgcacaaattcccggatatgacttcgtctccgggtggggaaggctgaatgtttttcatgcagccagcgtttttgagctgaacatgaaggttcatcccgttttaaaccgccatacggcagtgacaggcacagccaaaagcggtgtgacggtcaaaatcttgcgagggaagcaagtattggggacgggcacggccggaaaatcaggcgcgttttcagtgaaaattccggcccagaaggcggggcaagttcttcatgtcgcggcatcgggccatcaggcggaaacctcgctcagaaccgtcgtggaaaaagcgccgaaaaacccgtccgtcaaacgcatcacgaacaaagatactgccgtaacgggtagaacggcagccggctacacgatcaaagtgaaaaacgcgtccaaaaaagtgatcgcgcaaggcaaagcggatgcatccggagcgtttaaagtgaaaatcaacaagcaaaaagaatatgccgttttgtatgtctccgcatctgctgatgaccacagagaaagcggcgatgtcaaaatgacggtggctgacgtcatcccgccaggcgccccgaaagtttatcaggtttccgataaaagtacggtgattcagggaaaaacagaagcaaacgcgcaagtcagcgcaaaagctaaaggaaagacgatcgcttccggcaaagcgaacggaaaaggtgaatacaagctgaaaatcagcaggcaaaaagccggaaccgtcatcggtgtaacagcaaaggataaagccggaaatgtcagcaaagcgacagccgtcactgtgcttgacaaaaccccgccgtctgcgccgaaggtgaatccggtgacaaacaagagcacggccgttaaggggaaagcagaagcgaacgccgccatcatcgtcaaatcaggaaagaaaacgatcggaaccggcaaggccgataaaaaaggcgcgttttttgtcaaaataaaaaaacaaaaggcaaacaccgttttagcggtcactgcaaaagataaagccggcaatacgagcaaagtgagcaaaataaaagtcaaaaaggcaaaataa

SEQ ID NO: 101 - DNA, 2685 bp, Bacillus subtilis
  misc_feature 1..2685: note = CDS: wprA
  source 1..2685: mol_type = unassigned DNA
atgaaacgcagaaaattcagctcggttgtggcggcagtgcttatttttgcactgattttcagccttttttctccgggaaccaaagctgcagcggccggcgcgatcgatcaggcggctgctctggaaaacggcaaagagcagacaggcgccatgaaggagccggaacaggtgaaatggtacaaagtgaccccgggagcaacggatattcagaaaaactcacatatggcactgaccgtaaagagtgattcagtactgaatgtatctgtatatccaagtaaggaaaaagcgcttaaagatgagacgtttgagatgtaccgttctttcacagcggaggatggaaaaagcgaagtcatttttccatacgcgtggagcggcccttactatgtaaaagttgaatacctcggagaagaagaaccagaggacggcggaacggcagaagcagctgcagaagccaagtatacgattgggtataaaggcaccaaaaaacagccgtcagatttagaagaggaagaagcttgtccggttgaaatgagtgtcgatcagaagaaatcaggaaaaggcatcctggataagttgagatcgattcgtgatgagcagctgagccaaacagcagaaggcaaagaactgacaagcctttattacaaagcagcaccgtttatcgttgcaaagctcgcactcaataaaacagcaagaaatgaaatctatcaggatcttgtgacattaaagccgttatttgacgatgtgtcagaaaacggagcatcatcttcgtataaggtcactgaaaaggatcaaaaagcgatcaaccggctatatgataaagctttacaatcagtcccgtcattccttaaagaggagataaagaaacaagcggaccgactaaatatgaagcagctgcaaggcaaaacagcaggagccattttaacagaaaacaatattgcagcaaaaagtgaagttcagacaacaaaggttattttcaaggtgaaggacaataaaagcctctcatccgtacataatgaaatgaagggcttttctgcaagcgcgcaatcgaaaaaagacatatctaatgtgaaaaaggcaaagaaactgtttgacaatctgtattcatttgaacttccgaaagacgagaaacagaacggcgcatatacggcaagcgccaaaagggtcaaaagcgctgctgcgacactatccaagatgtccaatgtagagtttgcggaacccgtacaggaatacaaaagcttggcaaacgatattcagtacccttatcaatggccgcttaaaaacaacggtgaaaacggcggtgtcaaaaatgcggatgtgaaatatgagcctgccaacacattgctgtccaaacgcaagcttaacgatacactcattgcagtagtagacacaggcgtagacagcacgcttgccgatttaaaaggaaaagtaagaacagatctcggacacaattttgtcggacgaaataacaatgcaatggatgatcaggggcatgggacgcatgtcgcaggcattattgcagcccaaagcgataacggctattcaatgactggattgaatgccaaagcaaaaatcatccctgtaaaagtgcttgattccgcaggttccggagatactgaacaaattgctctcggcatcaaatatgctgctgacaaaggagcaaaggtgattaatttaagtttaggcggaggctacagccgcgtgcttgaatttgctttgaagtacgcagctgacaaaaatgtcttgattgccgcagccagcgggaatgatggagaaaatgccttatcttatcctgcatcttctaaatatgtgatgtcagtcggcgcaacgaatcgcatggatatgaccgctgatttctctaactatggaaaaggtctggacatctctgctccagggtctgatatcccgagcttagtgccgaacggaaatgtcacgtacatgagcggaacgtctatggcgacgccatatgctgccgccgctgcgggactgctgtttgctcaaaatcctaagctaaaaagaacagaagttgaggatatgttgaaaaagacggcagatgatatttcctttgaaagtgtcgatggcggagaagaagagttgtatgacgattatggcgatccgattgaaattccgaagacacctggtgtagactggcattcaggctacgggcggctgaatgtcatgaaggctgtcagcgcagctgatttacagcttaaggtcaacaagctggaaagcactcaaacagctgtcagaggaagcgcgaaggaaggcacccttatcgaggtgatgaacggcaaaaagaaactcggcagcgccaaagccggaaaagacaatgcgttcaaggtgaatatcgcgactcaaaaacaggatcaagtactgtatctgaaagcaacaaaaggcgatgcgaaaacatcgtataaagttgttgtcgtcaaaggaaaaccttccggcacaccgaaagtaaacgcggtgaaaacgaaggatacggcagtaaaagggaaggcaaacagcaaagcgatgatcagagtgaaaaacaaatcaaagaaagtcattgcttctgccaaagctgacgcaaaaggaacgttttcggtgaaaatcaaaaaacaaaaagccggaacggtgctgtacgtcacggctgtggatacagataaaaaagaaagcaaggaagcaaaagttgttgttgaaaagtaa

SEQ ID NO: 102 - DNA, 960 bp, Bacillus subtilis
  misc_feature 1..960: note = CDS: ispA
  source 1..960: mol_type = unassigned DNA
atgaatggtgaaatccgcttgatcccgtatgtgacgaatgagcagatcatggatgtaaacgagcttcctgagggcattaaagtgattaaggcgccagaaatgtgggcaaaaggggtaaaaggcaaaaatattaaagttgctgtattagacacaggctgcgacacaagccaccctgatttaaaaaatcaaatcatcggcggaaagaacttcacggatgacgacggcggcaaggaagatgcgatttccgactacaacggacacggcacacacgtcgccggaacaattgcagctaatgattcaaacggaggcattgccggagtcgcgcctgaggcaagcctattgattgtgaaggttcttggcggcgagaacggcagcggacaatacgaatggataattaacggcatcaactacgcggtggaacagaaagtcgacatcatctcaatgtcccttggagggccaagcgatgtgccagagctaaaagaagcagtgaaaaacgcagtgaaaaacggagtgcttgtcgtttgtgcagcgggaaatgaaggtgacggcgacgaacgcacagaagagctttcctaccccgcagcttataatgaagtgattgcagttggatctgtttctgtagcgcgagaattatcagaattttctaacgcgaataaagagattgaccttgtggcaccaggagaaaacatcttatccacccttcccaacaagaagtacggtaagctgaccggcacttcaatggctgcccctcatgtcagcggtgcgcttgctttaatcaaaagctatgaagaagaatcatttcaaagaaagctttctgaatctgaggttttcgcacagctaatccgcaggacacttcctcttgatattgcaaaaacgctggcaggcaatggattcctgtatttaacagctcctgatgagctcgcagaaaaagcagagcaatcacatttgttgaccctataa

SEQ ID NO: 103 - DNA, 1329 bp, Bacillus subtilis
  misc_feature 1..1329: note = CDS: aprX
  source 1..1329: mol_type = unassigned DNA
atgtttgggtactctatggtacaaatggtgagagcaaatgctcacaagctagattggccgttacgggaaaccgtattgcaactttacaagcccttcaaatggactccttgcttcttgcataaatttttcgaaaccaaattgcaaaacagaaaaaaaatgtcagtcattatcgaatttgaggaaggctgtcacgagaccggctttcaaatggctggagaggtgctgcaaaaagaaaaacgcagcaagctaaaaagccgcttcaacaaaatcaactgctgcagcgccgaggttactccttcagcattacattcccttctctctgaatgcagcaatatccgtaaagtgtatttaaaccgcgaagtcaaagcgcttttagacacggcaaccgaagcaagccatgcgaaggaagtcgtcagaaacgggcaaaccctgacaggaaaaggtgttactgtcgctgttgtcgatacaggaatctacccgcatcctgatctagaaggaagaattatcggattcgcggacatggtcaatcaaaaaacagagccgtacgatgataatggtcatggcacacactgtgcaggtgatgtcgcaagcagcggtgcatcctcttccggtcaataccgcggacctgcaccggaagccaatctcatcggcgtaaaggtattaaacaagcagggttcaggaacgttagcagatattatagagggtgttgagtggtgtattcaatacaatgaggacaaccccgatgaaccgattgatattatgagcatgtcgctcgggggcgatgcccttagatatgatcatgaacaggaagatccgttagtcagagcggttgaggaagcgtggagcgcagggattgtcgtctgtgtcgctgccggaaactcaggccctgattcgcagaccattgcaagcccgggcgtgagcgaaaaggtgattacagtaggtgcgcttgacgataacaatactgcaagcagcgatgatgatactgtcgcttccttctcaagccgcggcccgacagtatacgggaaagaaaagcccgatattttagcgccgggtgttaatattatttcccttcgctctccgaattcttatattgataagctccaaaagtcaagccgtgtagggtctcagtactttacaatgtcaggaacctcgatggcaacaccgatttgcgcaggaatcgcagctttaattcttcagcaaaacccggatcttacccctgatgaagtaaaagagcttttgaaaaacggcacggacaaatggaaagatgaagatccgaatatctacggagcgggcgctgtcaacgcagaaaattccgttcccggccaataa

SEQ ID NO: 104 - DNA, 1329 bp, Bacillus licheniformis
  misc_feature 1..1329: note = CDS: aprX
  source 1..1329: mol_type = unassigned DNA
atgttcggttcctacatggttcaaatggtaaggtccaacgcccataagctggaccggccgctgagagaaagcgtgctgaaactctataaaccttttaaatggacgccttgctttttacacggttttttcgaaaaaatattaatgaaaaacaaaaaaataccagtcattattgaatttgaaaaagactgtcagcataaaggctatcaattagtcaatgaaatgatcggcagcaaaagaagaaacaaaatcaagcatcgcttctccaacgttagctgctgcagcgctgaagtcaccccttcttctttgcaatcgcttctatctgagtgcggcgacattcgcaaaatttatttaaatcgcaaagtaaaagcgctcctcgacgtagcggttgaatcaagccatgccaaagaagtggtcagaaacaatcagacgctgacaggcaaaggagtgacagtcgctgtcattgatacgggcgtataccctcacgaagatcttgaaggcaggatcagggcttttcaagactttatcaaccagagaacagaaccctatgatgacaatgggcacggcacacactgcgccggtgatgcttgcggaaacggagcggcctcatcgggtcagtaccgcggacctgctcctgaagcagaacttgtcggtgtaaaagtattggacaaaatgggatccggatcgctcgaaaccgtcattcaaggcgtagattggtgcattcaattcaataaggaaaatcctgatgatccgatcgacattatttcaatgtcattgggtgcagaagccttgcgctacgagaatgaagaagaagatccagtcgttaaagctgttcatgcagcatgggacgcaggcattgttgtatgtgcggcagccggcaactccggtcctgatgcgcaaacgattgccagcccgggtgtcagcagcaagattattacagtcggagccttggatgacagggatacagtcagccgggaggatgacgatgtcgcctcttattcaagcagagggccgacaatctatggtcaagtcaaaccggacttgctggtaccgggcacaaatattacgtcgcttcgttcacccggatcttttctcgataagctgcaaaaaacaaacagagtcggcacaaaatatatgacattgtccggaacctcgatggctacgccgatctgcgcaggaattgccgcacttatccttcagcaagccccgggcacagaacctgatgaagtcaaacagctgctaatggacggtaccgatttatggaaagatcgcgatccaaatgtttacggtgcagggtacatcaacgcagaacaatcggtgccgcaagactaa

SEQ ID NO: 105 - protein, 379 aa, Bacillus licheniformis
  REGION 1..379: note = protein: AprE
MMRKKSFWLGMLTALMLVFTMAFSDSASAAQPAKNVEKDYIVGFKSGVKTASVKKDIIKESGGKVDKQFRIINAAKAKLDKEALEEVKNDPDVAYVEEDHVAHALAQTVPYGIPLIKADKVQAQGYKGANVKVAVLDTGIQASHPDLNVVGGASFVAGEAYNTDGNGHGTHVAGTVAALDNTTGVLGVAPNVSLYAVKVLNSSGSGSYSGIVSGIEWATTNGMDVINMSLGGPSGSTAMKQAVDNAYARGVVVVAAAGNSGSSGNTNTIGYPAKYDSVIAVGAVDSNSNRASFSSVGAELEVMAPGAGVYSTYPTSTYATLNGTSMASPHVAGAAALILSKHPNLSASQVRNRLSSTATYLGSSFYYGKGLINVEAAAQ

SEQ ID NO: 106 - protein, 381 aa, Bacillus subtilis
  REGION 1..381: note = protein: AprE
MRSKKLWISLLFALTLIFTMAFSNMSAQAAGKSSTEKKYIVGFKQTMSAMSSAKKKDVISEKGGKVQKQFKYVNAAAATLDEKAVKELKKDPSVAYVEEDHIAHEYAQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 107 - protein, 1432 aa, Bacillus licheniformis
  REGION 1..1432: note = protein: Bpr
MKRKLRKKAFSTILSGLLIGSLFMPAVSDAAAKPALTSMKEQAAAGKGKISKTLVKQFKKEDQVTFLIKLKDQVDTPKVAKQAEKNAKKKSLSAAKTEYQKRSAVVSALRVKADESQSDLKRYLKKQEKQGDVKKIRSYYIVNGMAVHATKEVMEQVAAFPEVEKVLPNEKRQLIKPTKQSVKKSAAKDEKEIEWNINRVDAPKAWKLGYDGSGTVVASIDTGVQWDHPALKEKYRGYDPQHPDQPNHEFSWYDAVSGASEPFDDLEHGTHVTGTMVGSEPDGQNQIGVAPGAKWIAVKAFSDDGGTDEDLIAAGEWILAPKDKDGNPHPEMAPDVVNNSWSGGAGIDEFYRDIVKAWRAAGIFPEFSAGNVDFANPGGPASIANPANYPEAFATGATDIDNRLADFSLQGPSPYDETKPEISAPGVNIRSSVPGSGYQDGWDGTSMAGPHVAAAAALIKQADSSITVDETEKILMETATPLTDSKFTESPNNGYGHGLVNVFDAVSAVTDGLGRAEGQVGKEGEDKSPPALNHQEITEIYSGSETPLKADVEDDVSIISVKLSYKTDDADWETIAAKQTSGDYKKGTYEAAVTVSEGKTLSYKWIVTDFGGNKTESKVYEVPISPAVTTGYKQDFENSASGWLTSGVKNSWERGIPQSGPNAAASGKNVFATNLTGPYESSANMNLLMPPVSVPKNQKLYLTYKYWRDIEEDFDYGFVYVQPEGKGEWIPAAEYSGKTSEWKDGQIDLSEYGGQTIKVMFNLQSDDSIEGDGLYIDDVALVKEVKSAGTKKRLGVEKQPAKMKDKKTKKRMIDPKKAKPAEALQEKTETKKAAPAVLPVRAQVSVLETGKSTYSNQATGAYSLAHAPGTYTLKAEAYGYESTAQTVKIESDKTTTADFVLKELKKGTLTGTIKNKKTGEPVRHAKLYIVEDAAVKPVQTDDDGSYSLTAYEGSYTVKVSANGYYSSEFSVDLKGDVSKDIDLDPFIGYPGEIGYDDGTGENAWAFYESGNGLAVKMTLENGQEKAMLKGGLFKFWDTEFPDPGGTDFAVEVYDASGEKGSPGKKIAGPFKAEALRTGEWTTVDLGDEGIIVGKDFYLVYVQKEDLANSPGLATDEDGEYSGRNWQYTDGSWSKAPSDQGNFMIRALVDYELSVPVITSPKDGFITNQKNAVIEGTSSPNTTVHLFNGDEEAGTAETAADGTFSKEIPLNKGENVITAKSSSASGTTDASEPVRIVLDQKKPKLTIDTPESGSKLNKETVTVKGTVSDDHLESVHVNGKKAAVDNGEYSARIMLDNGKNEIKVTASDAAGNKTTKKVTVDVNFEAPQITGLKPAEDLELKTGETVKIEFESAADLDAVFVIRMPLTNFKTAAQNVTELPIREVSKGKYEGYWTATSTAKAKGAEIEVIVRDDYGNETRQTAKGKLYINEKLK

SEQ ID NO: 108 - protein, 1433 aa, Bacillus subtilis
  REGION 1..1433: note = protein: Bpr
MRKKTKNRLISSVLSTVVISSLLFPGAAGASSKVTSPSVKKELQSAESIQNKISSSLKKSFKKKEKTTFLIKFKDLANPEKAAKAAVKKAKSKKLSAAKTEYQKRSAVVSSLKVTADESQQDVLKYLNTQKDKGNADQIHSYYVVNGIAVHASKEVMEKVVQFPEVEKVLPNEKRQLFKSSSPFNMKKAQKAIKATDGVEWNVDQIDAPKAWALGYDGTGTVVASIDTGVEWNHPALKEKYRGYNPENPNEPENEMNWYDAVAGEASPYDDLAHGTHVTGTMVGSEPDGTNQIGVAPGAKWIAVKAFSEDGGTDADILEAGEWVLAPKDAEGNPHPEMAPDVVNNSWGGGSGLDEWYRDMVNAWRAADIFPEFSAGNTDLFIPGGPGSIANPANYPESFATGATDINKKLADFSLQGPSPYDEIKPEISAPGVNIRSSVPGQTYEDGWDGTSMAGPHVSAVAALLKQANASLSVDEMEDILTSTAEPLTDSTFPDSPNNGYGHGLVNAFDAVSAVTDGLGKAEGQVSVEGDDQEPPVYQHEKVTEAYEGGSLPLTLTAEDNVSVTSVKLSYKLDQGEWTEITAKRISGDHLKGTYQAEIPDIKGTKLSYKWMIHDFGGHVVSSDVYDVTVKPSITAGYKQDFETAPGGWVASGTNNNWEWGVPSTGPNTAASGEKVYGTNLTGNYANSANMNLVMPPIKAPDSGSLFLQFKSWHNLEDDFDYGYVFVLPEGEKNWEQAGVYNGKTSSWTDEEIDLSAYKGQNIQVMFNLQSDESIAKEGWYIDDVVLSDKSAGKTVKKNKLGVEKPSGKQKKKPVNPKKAKPSANTAVKHQNKAIQPQVLPLKAQVSVVETGKSTYSDQSTGQYTLKHKAGDYTLMAEAYGYQSKTQKVSLKTDQTTQANFTLEEMKKGTLKGTVINKTTGEPVTGASVYVVEDAAVEPAMTNDKGEYMLEAYEGAYTIKVAAPGYYSDEFSVELKGDVTKETALKPFVGYPGEIAYDDGTAENANSYFAAGNGWAVKMTLADGKDKGMLTGGLFRFWDTEFPDPGGTEFKVEVYDATGKDGAPGKKIAGPFNAEALRNGEWTKVDLSSKGIMVDKDFYLVYIQSKPDPYSPGLAMDETGQNSGRNWQYIDGKWQPGDKADGNYMIRALVDYEAAVPEITSPTDKSYTNKDSVTVKGNASPGTTVHIYNGEKEAGETKAAADGTFHAGIILNKGENELTATASTDNGTTDASSPITVTLDQEKPELTLDNPKDGGKTNKETLTVKGAVSDDNLKDVKVNGKKATVADGSYSARILLENGRNEIKVIATDLAGNKTTKKTVIDVNFDKPVISGLIPGEDKNLKAGESVKIAFSSAEDLDATFTIRMPLTNARASVQNATELPLREISPGRYEGYWTATSSIKAKGAKVEVIVRDDYGNETRKTANGKLNMNTEN

SEQ ID NO: 109 - protein, 591 aa, Bacillus licheniformis
  REGION 1..591: note = protein: Epr
MKKLWKIAVSAAMFVGFFANSPRIQAESNKQENEVIVVYKNTSGKETVIEQADTVEHVYRHIPAAAVTADDKTVRELEHDPDVLYVEDNLPVAAADSTALKAFSSSTAQNASAFSQWNIKLIQAALAWNKGLTGKQVKIAVIDSGISPHEELSIAGGASMVGYTASYRDDNGHGTHVAGIIGAKHNGRGIDGIAPGAQLYAVKALDRNGAGDLKGILQGIDWSIQHGIDIINMSLVVSGDSQVLHDAVDKAYKRGIILVGASGNAGNGKSVYYPAAYSSVIAVSATNEKNQIASFSNTGSAVEFSAPGTSIISTSSDRGYAIGSGTSQATPHVTGMFALLKQLYPTASNAELRKKMQFYTSDLGAPGRDHLFGYGLIRFKEVTQPLEKAQKAVGQAEKTKKKADIQTAQKAIEPLPADADKTALKKRLNTVKEQLKKTAESKVKLAEKQKKKTNADSAQKAVNELDSGTFKTNLQKRINAVRSSLLKTAKQAVAKAEKAATDSNLGKAQKAINELPAGKDKSNLQKRLNTAKKQAAAAYNKKVSAAKAKVKTAEQKRTKKTKSAAQSAVGKLKASAEKTKLQKRINAIKLK

SEQ ID NO: 110 - protein, 645 aa, Bacillus subtilis
  REGION 1..645: note = protein: Epr
MKNMSCKLVVSVTLFFSFLTIGPLAHAQNSSEKEVIVVYKNKAGKETILDSDADVEQQYKHLPAVAVTADQETVKELKQDPDILYVENNVSFTAADSTDFKVLSDGTDTSDNFEQWNLEPIQVKQAWKAGLTGKNIKIAVIDSGISPHDDLSIAGGYSAVSYTSSYKDDNGHGTHVAGIIGAKHNGYGIDGIAPEAQIYAVKALDQNGSGDLQSLLQGIDWSIANRMDIVNMSLGTTSDSKILHDAVNKAYEQGVLLVAASGNDGNGKPVNYPAAYSSVVAVSATNEKNQLASFSTTGDEVEFSAPGTNITSTYLNQYYATGSGTSQATPHAAAMFALLKQRDPAETNVQLREEMRKNIVDLGTAGRDQQFGYGLIQYKAQATDSAYAAAEQAVKKAEQTKAQIDINKARELISQLPNSDAKTALHKRLDKVQSYRNVKDAKDKVAKAEKYKTQQTVDTAQTAINKLPNGTDKKNLQKRLDQVKRYIASKQAKDKVAKAEKSKKKTDVDSAQSAIGKLPASSEKTSLQKRLNKVKSTNLKTAQQSVSAAEKKSTDANAAKAQSAVNQLQAGKDKTALQKRLDKVKKKVAAAEAKKVETAKAKVKKAEKDKTKKSKTSAQSAVNQLKASNEKTKLQKRLNAVKPKK

SEQ ID NO: 111 - protein, 316 aa, Bacillus licheniformis
  REGION 1..316: note = protein: Mpr
MVSKKSVKRGLITGLIGISIYSLGMHPAQAAPSPHTPVSSDPSYKAETSVTYDPNIKSDQYGLYSKAFTGTGKVNETKEKAEKKSPAKAPYSIKSVIGSDDRTRVTNTTAYPYRAIVHISSSIGSCTGWMIGPKTVATAGHCIYDTSSGSFAGTATVSPGRNGTSYPYGSVKSTRYFIPSGWRSGNTNYDYGAIELSEPIGNTVGYFGYSYTTSSLVGTTVTISGYPGDKTAGTQWQHSGPIAISETYKLQYAMDTYGGQSGSPVFEQSSSRTNCSGPCSLAVHTNGVYGGSSYNRGTRITKEVFDNLTNWKNSAQ

SEQ ID NO: 112 - protein, 313 aa, Bacillus subtilis
  REGION 1..313: note = protein: Mpr
MKLVPRFRKQWFAYLTVLCLALAAAVSFGVPAKAAENPQTSVSNTGKEADATKNQTSKADQVSAPYEGTGKTSKSLYGGQTELEKNIQTLQPSSIIGTDERTRISSTTSFPYRATVQLSIKYPNTSSTYGCTGFLVNPNTVVTAGHCVYSQDHGWASTITAAPGRNGSSYPYGTYSGTMFYSVKGWTESKDTNYDYGAIKLNGSPGNTVGWYGYRTTNSSSPVGLSSSVTGFPCDKTFGTMWSDTKPIRSAETYKLTYTTDTYGCQSGSPVYRNYSDTGQTAIAIHTNGGSSYNLGTRVTNDVFNNIQYWANQ

SEQ ID NO: 113 - protein, 538 aa, Bacillus subtilis
  REGION 1..538: note = protein: NprB
MRNLTKTSLLLAGLCTAAQMVFVTHASAEESIEYDHTYQTPSYIIEKSPQKPVQNTTQKESLFSYLDKHQTQFKLKGNANSHFRVSKTIKDPKTKQTFFKLTEVYKGIPIYGFEQAVAMKENKQVKSFFGKVHPQIKDVSVTPSISEKKAIHTARRELEASIGKIEYLDGEPKGELYIYPHDGEYDLAYLVRLSTSEPEPGYWHYFIDAKNGKVIESFNAIHEAAGTGIGVSGDEKSFDVTEQNGRFYLADETRGKGINTFDAKNLNETLFTLLSQLIGYTGKEIVSGTSVFNEPAAVDAHANAQAVYDYYSKTFGRDSFDQNGARITSTVHVGKQWNNAAWNGVQMVYGDGDGSKFKPLSGSLDIVAHEITHAVTQYSAGLLYQGEPGALNESISDIMGAMADRDDWEIGEDVYTPGIAGDSLRSLEDPSKQGNPDHYSNRYTGTEDYGGVHINSSIHNKAAYLLAEGGVHHGVQVEGIGREASEQIYYRALTYYVTASTDFSMMKQAAIEAANDLYGEGSKQSASVEKAYEAVGIL

SEQ ID NO: 114 - protein, 521 aa, Bacillus subtilis
  REGION 1..521: note = protein: NprE
MGLGKKLSVAVAASFMSLSISLPGVQAAEGHQLKENQTNFLSKNAIAQSELSAPNDKAVKQFLKKNSNIFKGDPSKRLKLVESTTDALGYKHFRYAPVVNGVPIKDSQVIVHVDKSDNVYAVNGELHNQSAAKTDNSQKVSSEKALALAFKAIGKSPDAVSNGAAKNSNKAELKAIETKDGSYRLAYDVTIRYVEPEPANWEVLVDAETGSILKQQNKVEHAAATGSGTTLKGATVPLNISYEGGKYVLRDLSKPTGTQIITYDLQNRQSRLPGTLVSSTTKTFTSSSQRAAVDAHYNLGKVYDYFYSNFKRNSYDNKGSKIVSSVHYGTQYNNAAWTGDQMIYGDGDGSFFSPLSGSLDVTAHEMTHGVTQETANLIYENQPGALNESFSDVFGYFNDTEDWDIGEDITVSQPALRSLSNPTKYNQPDNYANYRNLPNTDEGDYGGVHTNSGIPNKAAYNTITKLGVSKSQQIYYRALTTYLTPSSTFKDAKAALIQSARDLYGSTDAAKVEAAWNAVGL

SEQ ID NO: 115 - protein, 802 aa, Bacillus licheniformis
  REGION 1..802: note = protein: Vpr
MRKSIVRYFVMAFILLFALSTFLTGVQATSVPDKKSPELEKAEIYGDIDVTSDKQTTVIVELKEKSLAEAKADGEKQTKASLKTARSKALKTLKKAKVNREYDRVFSGFSMKLPASEIPKLLAVKEVKAVYPNATYKPDSVKGKDVTLAADAIYPQMDKSAPFIGADQAWKSGYTGKGIKVAVIDTGVDYTHPDLKNNFGPYKGYDFVDNDYDPQETPTGDPRGGATDHGTHVAGTIAANGQIKGVAPEATLLAYRVLGPGGSGTTENVIAGIEKAVADGAKVMNLSLGNSLNSPDYATSIALDWAMAEGVVAVTSNGNSGPENWTVGSPGTSRVAISVGASQLPYNEYSVTLPSYSSAKVMGYQEEKDLEALNGQEVELVEAGLGQADDFSGKDVKGKVAVIQRGVIPFVDKAENAKNAGAIGAVIYNNATGEIEANVMGMAVPTVKLSKEEGEKLVQQIKEGKHSVVFSFKLDKKLGETIASFSSRGPVMDTWMIKPDVSAPGVNIVSTIPTHDPKNPYGYGSKQGTSMASPHVAGTAAILKQAKPDWTPEQIKGVLMNTAEKLTDENGKPLPHNTQGAGSIRIMEALKASSIVTPGSHSYGTFLKDKGKQTKKQAFTIENLSSHRKAYQLEYSFKGTGITVSGTERVVVPANQTGKAAAKVTVNSAKTKAGTYEGTVYIREDGRKVAEIPLLLIVKEPDYPRVTSVTVEPGAKQGAYTIEAYLPGGAEELAFLVYDENLNLLGQAGVYKNQGKGYQSYQWNGKINDAASLKSGKYYMLAYASAKGKSSYVLTEDPFIVE

SEQ ID NO: 116 - protein, 806 aa, Bacillus subtilis
  REGION 1..806: note = protein: Vpr
MKKGIIRFLLVSFVLFFALSTGITGVQAAPASSKTSADLEKAEVFGDIDMTTSKKTTVIVELKEKSLAEAKEAGESQSKSKLKTARTKAKNKAIKAVKNGKVNREYEQVFSGFSMKLPANEIPKLLAVKDVKAVYPNVTYKTDNMKDKDVTISEDAVSPQMDDSAPYIGANDAWDLGYTGKGIKVAIIDTGVEYNHPDLKKNFGQYKGYDFVDNDYDPKETPTGDPRGEATDHGTHVAGTVAANGTIKGVAPDATLLAYRVLGPGGSGTTENVIAGVERAVQDGADVMNLSLGNSLNNPDWATSTALDWAMSEGVVAVTSNGNSGPNGWTVGSPGTSREAISVGATQLPLNEYAVTFGSYSSAKVMGYNKEDDVKALNNKEVELVEAGIGEAKDFEGKDLTGKVAVVKRGSIAFVDKADNAKKAGAIGMVVYNNLSGEIEANVPGMSVPTIKLSLEDGEKLVSALKAGETKTTFKLTVSKALGEQVADFSSRGPVMDTWMIKPDISAPGVNIVSTIPTHDPDHPYGYGSKQGTSMASPHIAGAVAVIKQAKPKWSVEQIKAAIMNTAVTLKDSDGEVYPHNAQGAGSARIMNAIKADSLVSPGSYSYGTFLKENGNETKNETFTIENQSSIRKSYTLEYSFNGSGISTSGTSRVVIPAHQTGKATAKVKVNTKKTKAGTYEGTVIVREGGKTVAKVPTLLIVKEPDYPRVTSVSVSEGSVQGTYQIETYLPAGAEELAFLVYDSNLDFAGQAGIYKNQDKGYQYFDWDGTINGGTKLPAGEYYLLAYAANKGKSSQVLTEEPFTVE

SEQ ID NO: 117 - protein, 1019 aa, Bacillus licheniformis
  REGION 1..1019: note = protein: WprA
MAAEDSVPNNETTLTSASPVEASFQSDDEVHWYKVNPSNQEIANYTHFRVKLKSDAELNISVYSSLENATGHQTFDRYNGYSYENNPALIDFPIAWKGPYYIKVENHHDEENETTSITDISYTISYEGVTLPPSIQEAEEECPAELSVSERETGKGILKQLRTIRDEVLSKTEKGKELSSLYYKAAPFISAKMLFNKSMRDSVYKDLVQLKPLFADVAKNGQVSAYSITNDDQKAISRLYETARASVPEPLKKQLDQVAKDIGIEQLTGSKVSAVLEKAGMATASSSAPENRYIVKLKEGKKPGSFKSKAQSSGVQALEPLGKSKTAFKDMYVVEMKESRSSGFKAAAKQYQAAASKIAKMPEVEFVEQVQQYEALSRDTQYPYQWSLKNNGKNRAANADIQFEQLQKLMKGKKLKDTVIAVVDTGVDHTLADLSGSVKKDEGYNYVGRTADAMDDNGHGTHVSGIIAAAQDNHFSMAGINAYAKILPVKVLDSSGSGDTEQIANGIIYAADHGAKVINLSLGGPYSRVMEYALKYAASKNVTIVAATGNDGVSEISYPASSKYTLSVGATNNLDLVSDYSNYGKGLDMVAPGTDIPSLVPDGNVTYMSGTSMAAPHVAAAAGLLLSQNPSLKPKQIASLLTETTADVAFEEQDNPNPDYDLDIEPAAQIPGYDFVSGWGRLNVFHAASVFELNMKVHPVLNRHTAVTGTAKSGVTVKILRGKQVLGTGTAGKSGAFSVKIPAQKAGQVLHVAASGHQAETSLRTVVEKAPKNPSVKRITNKDTAVTGRTAAGYTIKVKNASKKVIAQGKADASGAFKVKINKQKEYAVLYVSASADDHRESGDVKMTVADVIPPGAPKVYQVSDKSTVIQGKTEANAQVSAKAKGKTIASGKANGKGEYKLKISRQKAGTVIGVTAKDKAGNVSKATAVTVLDKTPPSAPKVNPVTNKSTAVKGKAEANAAIIVKSGKKTIGTGKADKKGAFFVKIKKQKANTVLAVTAKDKAGNTSKVSKIKVKKAK

SEQ ID NO: 118 - protein, 894 aa, Bacillus subtilis
  REGION 1..894: note = protein: WprA
MKRRKFSSVVAAVLIFALIFSLFSPGTKAAAAGAIDQAAALENGKEQTGAMKEPEQVKWYKVTPGATDIQKNSHMALTVKSDSVLNVSVYPSKEKALKDETFEMYRSFTAEDGKSEVIFPYAWSGPYYVKVEYLGEEEPEDGGTAEAAAEAKYTIGYKGTKKQPSDLEEEEACPVEMSVDQKKSGKGILDKLRSIRDEQLSQTAEGKELTSLYYKAAPFIVAKLALNKTARNEIYQDLVTLKPLFDDVSENGASSSYKVTEKDQKAINRLYDKALQSVPSFLKEEIKKQADRLNMKQLQGKTAGAILTENNIAAKSEVQTTKVIFKVKDNKSLSSVHNEMKGFSASAQSKKDISNVKKAKKLFDNLYSFELPKDEKQNGAYTASAKRVKSAAATLSKMSNVEFAEPVQEYKSLANDIQYPYQWPLKNNGENGGVKNADVKYEPANTLLSKRKLNDTLIAVVDTGVDSTLADLKGKVRTDLGHNFVGRNNNAMDDQGHGTHVAGIIAAQSDNGYSMTGLNAKAKIIPVKVLDSAGSGDTEQIALGIKYAADKGAKVINLSLGGGYSRVLEFALKYAADKNVLIAAASGNDGENALSYPASSKYVMSVGATNRMDMTADFSNYGKGLDISAPGSDIPSLVPNGNVTYMSGTSMATPYAAAAAGLLFAQNPKLKRTEVEDMLKKTADDISFESVDGGEEELYDDYGDPIEIPKTPGVDWHSGYGRLNVMKAVSAADLQLKVNKLESTQTAVRGSAKEGTLIEVMNGKKKLGSAKAGKDNAFKVNIATQKQDQVLYLKATKGDAKTSYKVVVVKGKPSGTPKVNAVKTKDTAVKGKANSKAMIRVKNKSKKVIASAKADAKGTFSVKIKKQKAGTVLYVTAVDTDKKESKEAKVVVEK

SEQ ID NO: 119 - protein, 319 aa, Bacillus subtilis
  REGION 1..319: note = protein: IspA
MNGEIRLIPYVTNEQIMDVNELPEGIKVIKAPEMWAKGVKGKNIKVAVLDTGCDTSHPDLKNQIIGGKNFTDDDGGKEDAISDYNGHGTHVAGTIAANDSNGGIAGVAPEASLLIVKVLGGENGSGQYEWIINGINYAVEQKVDIISMSLGGPSDVPELKEAVKNAVKNGVLVVCAAGNEGDGDERTEELSYPAAYNEVIAVGSVSVARELSEFSNANKEIDLVAPGENILSTLPNKKYGKLTGTSMAAPHVSGALALIKSYEEESFQRKLSESEVFAQLIRRTLPLDIAKTLAGNGFLYLTAPDELAEKAEQSHLLTL

SEQ ID NO: 120 - protein, 442 aa, Bacillus subtilis
  REGION 1..442: note = protein: AprX
MFGYSMVQMVRANAHKLDWPLRETVLQLYKPFKWTPCFLHKFFETKLQNRKKMSVIIEFEEGCHETGFQMAGEVLQKEKRSKLKSRFNKINCCSAEVTPSALHSLLSECSNIRKVYLNREVKALLDTATEASHAKEVVRNGQTLTGKGVTVAVVDTGIYPHPDLEGRIIGFADMVNQKTEPYDDNGHGTHCAGDVASSGASSSGQYRGPAPEANLIGVKVLNKQGSGTLADIIEGVEWCIQYNEDNPDEPIDIMSMSLGGDALRYDHEQEDPLVRAVEEAWSAGIVVCVAAGNSGPDSQTIASPGVSEKVITVGALDDNNTASSDDDTVASFSSRGPTVYGKEKPDILAPGVNIISLRSPNSYIDKLQKSSRVGSQYFTMSGTSMATPICAGIAALILQQNPDLTPDEVKELLKNGTDKWKDEDPNIYGAGAVNAENSVPGQ

SEQ ID NO: 121 - protein, 317 aa, Bacillus licheniformis
  REGION 1..317: note = protein: IspA
MQMRLIPYVTDTKLMDVNELPEGIDAIKAPELWSQGFKGKDITVAVLDTGCDATHPDLADRIIGGRNFTDDDNGKEDQFHDYNGHGTHVAGTIAANDQNGGISGVAPEANLLIVKVLGGEDGSGDYEWIINGINYAVEQKADIISMSLGGPADVPELKEAVTNAVKSGVLVVCAAGNEGDGNDRTEEYSYPAAYNEVIAVGSVSLTRESSEFSNANKEIDLVAPGEEILSTLPDHQYGKLTGTSMATPHVSGALALIKSAEEEAFKRKLTEPELYAQLIRRTLPLDYSKALIGNGFLYLSAPEVLAEKAGEAKLLSL

SEQ ID NO: 122 - protein, 442 aa, Bacillus licheniformis
  REGION 1..442: note = protein: AprX
MFGSYMVQMVRSNAHKLDRPLRESVLKLYKPFKWTPCFLHGFFEKILMKNKKIPVIIEFEKDCQHKGYQLVNEMIGSKRRNKIKHRFSNVSCCSAEVTPSSLQSLLSECGDIRKIYLNRKVKALLDVAVESSHAKEVVRNNQTLTGKGVTVAVIDTGVYPHEDLEGRIRAFQDFINQRTEPYDDNGHGTHCAGDACGNGAASSGQYRGPAPEAELVGVKVLDKMGSGSLETVIQGVDWCIQFNKENPDDPIDIISMSLGAEALRYENEEEDPVVKAVHAAWDAGIVVCAAAGNSGPDAQTIASPGVSSKIITVGALDDRDTVSREDDDVASYSSRGPTIYGQVKPDLLVPGTNITSLRSPGSFLDKLQKTNRVGTKYMTLSGTSMATPICAGIAALILQQAPGTEPDEVKQLLMDGTDLWKDRDPNVYGAGYINAEQSVPQD

SEQ ID NO: 123 - protein, 317 aa, Bacillus sp.
  REGION 1..317: note = protein: IspA
MQMRLIPYVTDTKLMDVNELPEGIDAIKAPELWSQGFKGKDITVAVLDTGCDATHPDLADRIIGGRNFTDDDNGKEDQFHDYNGHGTHVAGTIAANDQNGGISGVAPEANLLIVKVLGGEDGSGDYEWIINGINYAVEQKADIISMSLGGPADVPELKEAVTNAVKNGVLVVCAAGNEGDGNDRTEEYSYPAAYNEVIAVGSVSLTRESSEFSNANKEIDLVAPGEEILSTLPDHQYGKLTGTSMATPHVSGALALIKSAEEEAFKRKLTEPELYAQLIRRTLPLDYSKALIGNGFLYLSAPEVLAEKAGEAKLLSL

SEQ ID NO: 124 - protein, 319 aa, Bacillus pumilus
  REGION 1..319: note = protein: IspA
MKGEIRLIPYEVKANVMEAKETPESIQEIKAPELWSSGFKGKGITIAVLDTGCDTEHPDLKDQIIGGKNFTDDDNGDAENVKDYNGHGTHVAGTIAATDQNGGILGVAPEAKLLIVKVLGGENGSGKYEWIINGINYAAEQKVDIISMSLGGPSNEPALQEAIQNAVKSGVLVVCAAGNEGDGDERTEEFSYPAAYNEVIAVGSVSLARESSEFSNANKEIDLVAPGEDILSTLPNHKYGRLTGTSMAAPHVSGALAIIKNAEEEAFQRKLSEPEVYAQLVRRTLPLKQSKALVGNGFLYLTAPDVLLEKTLEADLLSL

SEQ ID NO: 125 - protein, 319 aa, Bacillus megaterium
  REGION 1..319: note = protein: IspA
MKSKVRIIPFQVVEQLNQFNEVPEGVALIGAEQVWKQTKGRGITIAVLDTGCDVTHQDLQERIIGGKNFTQDDQQNPDIFQDYNGHGTHVAGTIAAAHNGTGVVGVAPEVGLLILKVLDGNGSGQYEWIIQGINYAIEQKVDIISMSLGGPSDVPELHEAIQDAVDQNILVVCAAGNEGDGEGSTDEFAYPGSYNEVITVGAVDLEKRSSVFSNSNNEVDVVAPGEKIVSTYLNGKYAALSGTSMATPHVAGALGLIKVLSNEMFGRELTEPELYAQLIRRTLPLNESPREVGNGFVHLTVVEKLLREIQSQMTSEVIR

SEQ ID NO: 126 - protein, 316 aa, Bacillus thuringiensis
  REGION 1..316: note = protein: IspA
MNSSTANKQKGIQLIPFTVDKVVEQVNEIPPGVQLIHAPQVWEKSAKGKDIVVAVLDTGCDMNHIDLKDRIIGGRNFTKDYEGDPNIYLDNNGHGTHVAGTIAATENSVGVLGVAPLAKMLVLKVLAGDGSGSYEQIIEAIHYAVNWRGPNKERVRVISMSLGGPQDVPELHEAIQNAVKQDVLVVCAAGNNGDCNDNTEELDFPGAYSEVIEVGAVNLERKIACFSNSNQEIDLVAPGDEILSTYPEGKYAVLSGTSMATPHVAGALALLIKQCEREYGRKLSEPEIYAQLIKRTVPLGYERTSEGNGLIDLLKE

SEQ ID NO: 127 - protein, 320 aa, Bacillus methanolicus
  REGION 1..320: note = protein: IspA (B. methanolicus MGA3)
MKNYVHVIPHKLVEQLNEVNEIPTGVDIIQAPKIWEKTKGKGITVAVLDTGCDTDHPDLKDRIIAGRNFTKDDGGNPEIFEDYNGHGTHVAGTIAASLNNNGVAGVAPEANLLILKVLGKDGSGQYDWIINAIRYAIEQKVDIISMSLGGPSDIKELHDAIKEAVLTHHILVVCAAGNEGDGEYITDEFAYPGSYNEVISVGAVDLERHSSHFTNSNNQVDIVAPGEKILSTYLNGSYATLSGTSMATPHVSGSLALIKNLSKSAFERELTEPELYAQLIKRTVPLGNSPKLEGNGFIYLTLLDYVKKVFDEKALVELVK

SEQ ID NO: 128 - protein, 319 aa, Bacillus sp.
  REGION 1..319: note = protein: IspA (B. velezensis FZB42)
MNGEMHLIPYVTDEQIMDVNELPEGIKVIKAPELWAKGFKGKDIKIAVLDTGCDINHPDLKDRIVGGKNFTDDDGGKEDAISDYNGHGTHVSGTIAANDSNGGISGVAPEASLLIVKVLGGQNGSGKYEWIINGINYAVEQKADIISMSLGGPSDVPELKEAVTNAVKSGVLVVCAAGNEGDGDERTEELSYPAAYNEVIAVGSVSIARKSSEFSNANKEIDLVAPGENILSTLPNHKYGKLTGTSMAAPHVSGALALIKGLEQDAFQRTLSEAEVYAQLVRRTLPLDIAKTLAGNGFLYLDAPEELMERAEQAQLLSF

SEQ ID NO: 129 - protein, 319 aa, Bacillus amyloliquefaciens
  REGION 1..319: note = protein: IspA (B. amyloliquefaciens XH7)
MNGEMRLIPYVTDEQIMDVNELPEGIKVIKAPELWAKGFKGKDIKIAVLDTGCDVNHPDLKDRIIGGKNFTDDDGGKEDAFSDYNGHGTHVSGTIAANDSNGGISGVAPEASLLIVKVLGGQNGSGKYEWIINGINYAVEQKADIISMSLGGPSDVPELKEAVTNAVKSGVLVVCAAGNEGDGDERTEELSYPAAYNEVIAVGSVSVARKSSEFSNANKEIDLVAPGENILSTLPNHKYGKLTGTSMAAPHVSGALALIKGLEQASFQRTLSEAEVYAQLVRRTLPLDIAKTLAGNGFLYLDAPDVLMEKAEQAQLLSF

SEQ ID NO: 130 - protein, 316 aa, Bacillus cereus
  REGION 1..316: note = protein: IspA
MNSSTANKQKGIQLIPFTVDKVVEQVNEIPPGVQLIHAPQVWEKSAKGKDIVVAVLDTGCDMNHIDLKDRIIGGRNFTKDYEGDPNIYLDNNGHGTHVAGTIAATENGVGVLGVAPLAKMLVLKVLAGDGSGSYEQIIEAIHYAVNWRGPNKERVRVISMSLGGPQDVPELHEAIQNAVKQDVLVVCAAGNNGDCNDNTEELDFPGAYSEVIEVGAVNLERKIACFSNSNQEIDLVAPGDEILSTYPEGKYAVLSGTSMATPHVAGALALLIKQCEREYGRKLSEPEIYAQLIKRTVPLGYERTSEGNGLIDLLKE

SEQ ID NO: 131 - protein, 319 aa, Bacillus mojavensis
  REGION 1..319: note = protein: IspA
MNGEIRLIPYVTNEQIMDVNELPESIKVIKAPEVWAKGVKGKDVKIAVLDTGCDTSHPDLKNQIIGGKNFTDDDGGKEDAISDYNGHGTHVAGTIAANDSNGGIAGVAPEASLLIVKVLGGENGSGKYEWIINGINYAVEQKADIISMSLGGPSDVPELQEAVKNAVKNGVLVVCAAGNEGDGDERTEELSYPAAYNDVIAVGSVSVARESSEFSNANKEIDLVAPGENILSTLPNKKYGKLTGTSMAAPHVSGALALIKSFEEESFQRELSESELYAQLIRRTLPLDIAKTLAGNGFLYLTAPDELSEKAEQSHLLTL

(19) United States
(12) Patent Application Publication
(10) Pub. No.: US 2025/0051822 A1
(43) Pub. Date: Feb. 13, 2025

(54) METHOD FOR IMPROVED PRODUCTION OF INTRACELLULAR PROTEINS IN BACILLUS

(71) Applicant: BASF SE, Ludwigshafen (DE)

(72) Inventors: Max Fabian Felle, Ludwigshafen (DE); Christopher Sauer, Ludwigshafen (DE); Mathis Appelbaum, Ludwigshafen (DE); Stefan Jenewein, Ludwigshafen (DE); Sebastian Schoof, Ludwigshafen (DE); Christian Riedele, Eppelheim (DE)

(21) Appl. No.: 18/720,123

(22) PCT Filed: Dec. 19, 2022
(86) PCT No.: PCT/EP2022/086747

§ 371 Date: Jun. 14, 2024

(30) Foreign Application Priority Data
Dec. 20, 2021 (EP) 21215959.4

(51) Int. Cl. C12P 21/02; C12N 9/38; C12N 15/75

(52) U.S. Cl. CPC C12P 21/02; C12N 9/2471; C12N 15/75; C12Y 302/01023

(57) ABSTRACT

The present invention relates to a method for producing at least one polypeptide of interest in a Bacillus host cell, said method comprising the steps of a) providing a Bacillus host cell comprising a polynucleotide encoding the at least one polypeptide of interest, b) cultivating the Bacillus host cell under conditions which allow for intracellular accumulation of said at least one polypeptide of interest, thereby producing said at least one polypeptide of interest, c) stop of aeration and addition of feed, and d) incubating the cells for at least hours. Moreover, the present invention relates to a Bacillus host cell comprising a polynucleotide encoding at least one polypeptide of interest lacking a functional secretion sequence, wherein the ispA gene has been inactivated in the Bacillus host cell.

### FIELD OF THE INVENTION

The present invention relates to a method for producing at least one polypeptide of interest in a Bacillus host cell, said method comprising the steps of a) providing a Bacillus host cell comprising a polynucleotide encoding the at least one polypeptide of interest, b) cultivating the Bacillus host cell under conditions which allow for intracellular accumulation of said at least one polypeptide of interest, thereby producing said at least one polypeptide of interest, c) stop of aeration and addition of feed, and d) incubating the cells for at least five hours. Moreover, the present invention relates to a Bacillus host cell comprising a polynucleotide encoding at least one polypeptide of interest lacking a functional secretion sequence, wherein the ispA gene has been inactivated in the Bacillus host cell.

### BACKGROUND

Microorganisms of the Bacillus genus are widely applied as industrial workhorses for the production of valuable compounds, such as chemicals, polymers and proteins, in particular proteins like washing- and/or cleaning-active enzymes or enzymes used for feed and food applications or biofuel production processes. The biotechnological production of these useful substances is conducted via fermentation of such Bacillus species and subsequent purification of the product. Bacillus species have been extensively used for production of secreted proteins either native or foreign to the host due to their high secretion capacity of proteins into the fermentation broth (Schallmey et al., Can J Microbiol. 2004; 50(1):1-17). This allows a simple product purification process compared to intracellular production. However, industrial relevant high production of secreted proteins is often limited to proteins that are naturally secreted.

Next to secreted proteins, intracellular production of high amounts of proteins in Bacillus species have been conducted. The production of various Cry-Toxins within Bacillus thuringiensis species are well known examples. Furthermore, WO 1990/09448 A1 describes a method for intracellular expression of homologous or heterologous proteins in Bacillus subtilis by use of a non-functional secretion signal peptide.

Methods for cell disruption and release of intracellularly produced proteins from microbial host cells are well described in the art. Enzymatic methods apply for example lysozyme or protease treatment to destabilize the bacterial cell wall. Other methods are thermal methods, like freezing-thawing, heating, or osmotic driven lysis by low-osmolarity solution, or mechanic disruption methods, like homogenizers, ultrasonication, pressing for example pressing using a ‘French Press’, or chemical methods such as the use of detergents, acid, base, or chaotropic reagents.

These methods represent an additional separate step during the downstream processing, additional costs (e.g. enzymes, devices) and undesirable contamination (e.g. added enzymes), or conditions that are detrimental to the stability of the protein of interest.

Hori et al (Hori, K., Unno, H.; Appl Microbiol Biotechnol 59, 211-216 (2002)) describes a self-disruptive Bacillus megaterium for polyhydroxybutyate production by introduction of a recombinant phage lysis system which is controlled during the fermentation process by inducer molecule dependent induction and glucose starvation which requires a specifically controlled fermentation process.

WO 2014/206829 A2 discloses a method for producing a natively secreted polypeptide, the method comprising the steps of providing a microorganism host cell comprising an exogenous polynucleotide encoding a natively secreted polypeptide without a translationally fused signal peptide; cultivating the microorganism host cell under conditions conducive to the expression of the polypeptide and, optionally, recovering the polypeptide. In Example 4, a Bacillus strain having a deletion of the ispA intracellular protease-encoding gene.

WO 2009/096916 A1 discloses a method of producing an SSI (Streptomyces Subtilisin Inhibitor) protein, comprising: culturing a host cell that has inactivated aprE, nprE, epr, ispA, bpr, vpr, wprA, mpr-ybj F and nprB genes. The SSI protein is fused to a celA signal sequence. The host cell secretes said SSI protein from said cell.

EP 3 805 384 A1 discloses a method for producing an acetolactate decarboxylase (ALDC) enzyme in a host cell comprising a genetic alteration that causes said host cell to produce a decreased amount of an endogenous extracellular serine protease (vpr) and/or a cell wall protease (wprA) and/or a neutral metalloprotease capable of clipping a sequence from a C-terminus of said ALDC enzyme. The host cell further may have decreased amounts of an endogenous major intracellular serine protease enzyme (IspA). The acetolactate decarboxylase enzyme comprises a signal peptide.

The intracellular production of proteins of thermophilic organisms in gram-negative mesophilic production hosts such as E. coli or Pseudomonas species are well known. Target proteins are recovered by cell lysis induced via heat treatment with temperatures >70 resulting recovery of the soluble target protein whereas denatured host cell proteins are separated (JP2615090; Olchon A, de Marco A.; BMC Biotechnol. 2007 Jan. 26; 7:7; Lapidot A, Shoham Y.; J Biotechnol. 1996 Nov. 15; 51(3):259-64.)

Bacillus cells are gram-positive. In gram-positive cells, the peptidoglycan (murein) layer is substantially thicker in Gram-positive bacteria (20 to 80 nm) than in Gram-negative bacteria (7 to 8 nm) which makes the purification of intracellularly produced polypeptides difficult.

The optimization of the Bacillus host cell for the intracellular production of biological compounds is of high relevance, where even small improvements in compound yield, e.g. increased yield in single process steps and/or in reduction of process steps are significant for production costs of compounds in large scale industrial production. Therefore, the present invention relates to a method for producing at least one polypeptide of interest in a Bacillus host cell, said method comprising cultivating the Bacillus host cell under conditions which allow for intracellular accumulation of said at least one polypeptide of interest and the subsequent lysis of the cell under mild conditions. Lysis is achieved by stop of aeration and addition of feed and incubating the cells for at least one hour at an increased temperature, such as a temperature of about 40° C. to 60° C., preferably about 45° C. to 55° C., preferably about 50° C. The cell may be a modified Bacillus host cell in which the ispA gene has been inactivated.

Therefore, the present invention relates to a method for producing one or more polypeptides of interest that are intracellularly accumulated in a Bacillus host cell. The polypeptide of interest can be released from the cell by mild lysis conditions. The present invention further relates to a modified Bacillus host cell with increased release of intracellularly accumulated biological compounds.

### BRIEF SUMMARY OF THE INVENTION

The present invention relates to a method for producing at least one polypeptide of interest in a Bacillus host cell, said method comprising the steps of
a) providing a Bacillus host cell comprising a polynucleotide encoding the at least one polypeptide of interest,
b) cultivating the Bacillus host cell under conditions which allow for intracellular accumulation of said at least one polypeptide of interest, thereby producing said at least one polypeptide of interest,
c) stop of aeration and addition of feed, and
d) incubating the Bacillus host cell for at least five hours.

The present invention also relates to a Bacillus host cell comprising a polynucleotide encoding at least one polypeptide of interest lacking a functional secretion sequence, wherein the ispA gene has been inactivated in the Bacillus host cell.

The present invention further relates to a method of producing a Bacillus host cell, comprising
a) providing a Bacillus cell comprising an ispA gene,
b) inactivating the ispA gene, and
c) introducing at least one polynucleotide encoding at least one polypeptide of interest into the cell.

In an embodiment, of the methods of the present invention, the at least one polypeptide of interest does not comprise a functional secretion sequence.

In an embodiment of the method for producing at least one polypeptide of interest, the Bacillus host cell is a ispA knock-out cell.

In an embodiment of the method for producing at least one polypeptide of interest, the host cell in step d) is incubated at an increased temperature, preferably, at a temperature of 40° C. to about 60° C., more preferably of about 42° C. to about 57° C., even more preferably of about 45° C. to about 55° C., and most preferably of about 50° C.

In an embodiment of the method for producing at least one polypeptide of interest, the cells are incubated in step d) for at least 10 hours.

In an embodiment of the method for producing at least one polypeptide of interest, the cells are incubated in step d) for 5 to 40 hours, preferably for 5 to 30 hours, even more preferably for 10 to 30 hours, and most preferably for 10 to 24 hours.

In an embodiment of the method for producing at least one polypeptide of interest, preferably at least 70%, more preferably at least 80, even more preferably at least 90%, or 100% of cells are lysed after d).

In an embodiment of the present invention, one or more of the following genes have been knocked-out in the Bacillus host cell: one or more extracellular proteases, the intracellular protease aprX, amylase, an amyloglucosidase, a cellulase, a chitinase, a lipase, a phospholipaseesterase, an invertase, an arylsulfatase, a mannanase, a pectinase and/or a pectate lyase.

In an embodiment of the present invention, the one or more extracellular proteases is/are aprE, mpr, bpr, vpr, epr, nprE, nprB and/or wprA, In an embodiment of the present invention, the at least one polypeptide of interest is selected from the group consisting of a B-galactosidase, xylanase, protease, phytase, casein, lactoferrin, dehydrogenase, nitrilase, transaminase, lactase, lipase and esterase.

In an embodiment of the present invention, the at least one polypeptide of interest is an enzyme used for food or feed production, such as a lactase, phytase, xylanase, mannanase, lipase, esterase, casein, lactoferrin, preferably lactase, casein or lactoferrin, most preferably a lactase.

In an embodiment of the present invention, the polypeptide of interest is a B-galactosidase.

In an embodiment of the present invention, the Bacillus host cell is a Bacillus alcalophilus, Bacillus amyloliquefaciens, Bacillus brevis, Bacillus cereus, Bacillus circulans, Bacillus clausii, Bacillus coagulans, Bacillus firmus, Bacillus globigii, Bacillus halodurans, Bacillus lautus, Bacillus lentus, Bacillus licheniformis, Bacillus paralicheniformis, Bacillus megaterium, Bacillus methanolicus, Bacillus methylotrophicus, Bacillus mojavensis, Bacillus pumilus, Geobacillus stearothermophilus (Bacillus stearothermophilus), Bacillus thuringiensis or Bacillus velezensis host cell, preferably a Bacillus licheniformis host cell.

In an embodiment of the present invention, the host cell is a Bacillus licheniformis host cell.

### DESCRIPTION OF THE FIGURES

FIG. 1 Release of ß-Galactosidase (EC 3.2.1.23) by cell lysis upon incubation under temperature controlled and shaken conditions in a thermomixer in 50 mL falcon tubes. The broth was transferred after the end of fermentation, pH was corrected to pH=7.0 with NH₄OH or H₂SO₄ and incubated at the indicated temperatures. Samples were withdrawn at the indicated timepoints and ß-Galactosidase (Lactase) activity was measured. The ß-Galactosidase activity values for each temperature were normalized to the values at t=0 h.

FIG. 2 The process of cell lysis is monitored by measuring the optical density. Declining optical density values measured at 600 nm indicates the lysis of bacterial cells. The incubation of fermentation broth was conducted in a small scale reactor system at a stirrer rate of 500 rpm, with pH-control (pH=7.0, NH₄OH or H₂SO₄) and temperature control at 30° C., 40° C. and 45° C. for the three batches. The values were normalized to the values at t=Oh.

FIG. 3 Release of intracellularly accumulated ß-Galactosidase into in the cultivation supernatant of the ispA knockout strain (BES #176) and a non-ispA knockout strain (BES #175) at different temperatures (30° C. and 50° C.).

### DETAILED DESCRIPTION OF THE INVENTION—DEFINITIONS

As set forth above, the present invention relates to a method for producing at least one polypeptide of interest in a Bacillus host cell, said method comprising the steps of
a) providing a Bacillus host cell comprising at least one polynucleotide encoding a polypeptide of interest,
b) cultivating the Bacillus host cell under conditions which allow for intracellular accumulation of said at least one polypeptide of interest, thereby producing said at least one polypeptide of interest,
c) stop of aeration and addition of feed, and
d) incubating the cells for about at least five hours, preferably at an increased temperature.

It is to be understood that as used in the specification and in the claims, “a” or “an” can mean one or more, depending upon the context in which it is used. Thus, for example, reference to “a cell” can mean that at least one cell can be utilized.

Further, it will be understood that the term “at least one” as used herein means one or more than one. For example two, three, four, five, six, seven, eight, nine, ten or more. Depending on the item the term refers to, the skilled person understands as to what upper limit the term may refer, if any.

The term “about” as used herein means that with respect to any number recited after said term an interval accuracy exists within which a technical effect can be achieved. Accordingly, “about” as referred to herein, preferably, refers to the precise numerical value or a range around said precise numerical value of ±20%, preferably ±15%, more preferably ±10%, and even more preferably ±5%. In an embodiment, the term refers to the exact value.

The term “comprising” as used herein shall not be understood in a limiting sense. The term rather indicates that more than the actual items referred to may be present, e.g., if it refers to a method comprising certain steps, the presence of further steps shall not be excluded. However, the term “comprising” also encompasses embodiments where only the items referred to are present, i.e. it has a limiting meaning in the sense of “consisting of”.

### Host Cell

In step a) of the method of the present invention, a host cell is provided comprising at least one polynucleotide encoding a polypeptide of interest. The host cell shall be a Bacillus host cell, i.e. a host cell belonging to the genus Bacillus.

For example, the Bacillus host cell may be a Bacillus alcalophilus, Bacillus amyloliquefaciens, Bacillus brevis, Bacillus cereus, Bacillus circulans, Bacillus clausii, Bacillus coagulans, Bacillus firmus, Bacillus globigii, Bacillus halodurans, Bacillus lautus, Bacillus lentus, Bacillus licheniformis, Bacillus paralicheniformis, Bacillus megaterium, Bacillus methanolicus, Bacillus methylotrophicus, Bacillus mojavensis, Bacillus pumilus, Geobacillus stearothermophilus (Bacillus stearothermophilus), Bacillus subtilis, Bacillus thuringiensis or Bacillus velezensis. In one embodiment, the bacterial host cell is a Bacillus amyloliquefaciens, Bacillus velezensis Bacillus lentus, Bacillus licheniformis, Bacillus pumilus, Bacillus stearothermophilus or Bacillus subtilis cell.

In a preferred embodiment, the host cell is a Bacillus licheniformis cell, Bacillus pumilus cell or a Bacillus subtilis cell.

In one embodiment, the host cell belongs to the species Bacillus licheniformis, such as a host cell of the Bacillus licheniformis strain as deposited under American Type Culture Collection number ATCC14580 (which is the same as DSM13, see Veith et al. “The complete genome sequence of Bacillus licheniformis DSM13, an organism with great industrial potential.” J. Mol. Microbiol. Biotechnol. (2004) 7:204-211). Alternatively, the host cell may be a host cell of Bacillus licheniformis strain ATCC31972. Alternatively, the host cell may be a host cell of Bacillus licheniformis strain ATCC53757. Alternatively, the host cell may be a host cell of Bacillus licheniformis strain ATCC53926. Alternatively, the host cell may be a host cell of Bacillus licheniformis strain ATCC55768. Alternatively, the host cell may be a host cell of Bacillus licheniformis strain DSM394. Alternatively, the host cell may be a host cell of Bacillus licheniformis strain DSM641.

Alternatively, the host cell may be a host cell of Bacillus licheniformis strain DSM1913. Alternatively, the host cell may be a host cell of Bacillus licheniformis strain DSM11259. Alternatively, the host cell may be a host cell of Bacillus licheniformis strain DSM26543.

Preferably, the Bacillus licheniformis strain is selected from the group consisting of Bacillus licheniformis ATCC 14580, ATCC 31972, ATCC 53757, ATCC 53926, ATCC 55768, DSM 13, DSM 394, DSM 641, DSM 1913, DSM 11259, and DSM 26543.

In another preferred embodiment, the host cell is a Bacillus velezensis host cell. For example, the host cell may be a host cell of the Bacillus velezensis strain FZB42.

In another preferred embodiment, the host cell is a Bacillus amyloliquefaciens host cell. For example, the host cell may be a host cell of the Bacillus amyloliquefaciens strain XH7.

In another preferred embodiment, the host cell is a Bacillus pumilus host cell. For example, the host cell may be a host cell of the Bacillus pumilus strain DSM27.

In another preferred embodiment, the host cell is a Bacillus lentus host cell. For example, the host cell may be a host cell of the Bacillus lentus strain DSM9.

In another preferred embodiment, the host cell is a Bacillus alcalophilus host cell. For example, the host cell may be a host cell of the Bacillus alcalophilus strain ATCC27647.

In another preferred embodiment, the host cell is a Bacillus methanolicus host cell. For example, the host cell may be a host cell of the Bacillus methanolicus strain PB1 (DSM16454) or Bacillus methanolicus strain MGA3 (ATCC53907).

### Polypeptide of Interest

The host cell, preferably, comprises at least one polynucleotide encoding a polypeptide of interest.

The terms “polynucleotide”, “nucleic acid sequence”, “nucleotide sequence”, “nucleic acid”, “nucleic acid molecule” are used interchangeably herein and refer to nucleotides, typically deoxyribonucleotides, in a polymeric unbranched form of any length. The terms “polypeptide” and “protein” are used interchangeably herein and refer to amino acids in a polymeric form of any length, linked together by peptide bonds.

The terms “coding for” and “encoding” are used interchangeably herein. Typically, the terms refer to the property of specific sequences of nucleotides in a polynucleotide, such as a gene, a cDNA, or an mRNA, to serve as templates for synthesis of other macromolecules such as a defined sequence of amino acids. Thus, a gene codes for a protein, if transcription and translation of mRNA corresponding to that gene produces the protein in a cell or other biological system.

The term “polypeptide of interest” as used herein refers to any protein, peptide or fragment thereof which is intended to be produced in the bacterial host cell. A protein, thus, encompasses polypeptides, peptides, fragments thereof as well as fusion proteins and the like.

In an embodiment, the polynucleotide encoding the polypeptide of interest can be native to the host cell. In an alternative embodiment, the polynucleotide encoding at least one polypeptide of interest is heterologous to the Bacillus host cell.

The term “native” (or wildtype or endogenous) cell or organism and “native” (or wildtype or endogenous) polynucleotide or polypeptide refers to the cell or organism as found in nature and to the polynucleotide or polypeptide in question as found in a cell in its natural form and genetic environment, respectively (i.e., without there being any human intervention).

The term “heterologous” (or exogenous or foreign or recombinant or non-native) polypeptide or protein as used throughout the specification is defined herein as a polypeptide or protein that is not native to the host cell. Similarly, the term “heterologous” (or exogenous or foreign or recombinant or non-native) polynucleotide refers to a polynucleotide that is not native to the host cell In an embodiment, the at least one polynucleotide encoding a polypeptide of interest is stably integrated into the bacterial chromosome. In an alternative embodiment, the at least one polynucleotide encoding a polypeptide of interest is present on a plasmid. The term “plasmid” refers to an extrachromosomal circular DNA, i.e. a vector that is autonomously replicating in the host cell. Thus, a plasmid is understood as extrachromosomal vector.

Preferably, the polypeptide of interest is an enzyme, such as a hydrolase.

In a particularly preferred embodiment, the enzyme is classified as an oxidoreductase (EC 1), a transferase (EC 2), a hydrolase (EC 3), a lyase (EC 4), an isomerase (EC 5), or a ligase (EC 6). In a preferred embodiment, the protein of interest is an enzyme suitable to be used in detergents, feed or food applications or biofuel production processes.

Most preferably, the enzyme is a hydrolase (EC 3), preferably, an esterase (EC 3.1), a glycosylase (EC 3.2) or a peptidase (EC 3.4). Especially preferred enzymes are enzymes selected from the group of carboxylic-ester hydrolases (EC 3.1.1), phosphoric-monoester hydrolases (EC 3.1.3), glycosidases (EC 3.2.1), protease (EC 3.4). Most preferred enzymes are enzymes selected from the group of a cellulase (EC 3.2.1.4), an endo-1,3-beta-xylanase (EC 3.2.1.32), an endo-1,4-beta-xylanase (EC 3.2.1.8), a lactase (EC 3.2.1.108), a galactosidase (EC 3.2.1.23 and EC 3.2.1.24), a mannanase (EC 3.2.1.24 and EC 3.2.1.25), a lipase (EC 3.1.1.3), a phytase (EC 3.1.3.8), a nuclease (EC 3.1.11 to EC 3.1.31) and a protease (EC 3.4); in particular an enzyme selected from the group consisting of protease, lipase, mannanase, phytase, xylanase, phosphatase, ß-galactosidase, lactase, glucoamylase, nuclease, and cellulase, preferably, xylanase, mannanase, lactase, ß-galactosidase or protease, preferably, a lactase, ß-galactosidase, lactoferrin (EC 3.1.21.1) and a protease. Most preferred is a ß-galactosidase (EC 3.2.1.23).

In a particularly preferred embodiment, the polypeptide of interest is a ß-galactosidase (EC 3.2.1.23).

In yet another embodiment the polypeptide of interest is casein such as alpha-casein, beta-casein or kappa-casein. In some embodiments the alpha-casein, beta-casein or kappa-casein is a mammalian polypeptide like ape, baboon, bear, buffalo, camel, cat, chimpanzee, cow, donkey, dog, echidna, elephant, fox, gibbon, goat, gorilla, guinea pig, horse, human, lemur, lion, macaque, mandrill, mountain goat, monkey, mouse, opossum, orangutan, panda, pig, rat, rabbit, sheep, squirrel, tiger, wallaby, whale, wolf, or woolly mammoth. In a more preferred embodiment the casein polypeptide is from human and bovine.

Further, the polypeptide of interest may be used in feed or food production. The lysis at elevated temperature is advantageous for such proteins, because it allows for the inactivation of many Bacillus proteins, in particular enzymatic side activities not limited to protease side activities, amylase side activities, amyloglucosidase side activities, cellulase side activities chitinase side activities, lipase side activities, phospholipaseesterase side activities, invertase side activities, arylsulfatase side activities, mannanase side activities, pectinases side activities, pectate lyase side activities.

In an embodiment of the present invention, the polypeptide of interest which is used in food production is selected from the group consisting of ß-galactosidase, casein, lactoferrin, and preferably ß-galactosidase.

In one preferred embodiment, the polypeptide of interest is intracellularly accumulated during cultivation of the Bacillus host cell. Thus, the polypeptide of interest is preferably not secreted by the Bacillus host cell. Thus, the polypeptide of interest, preferably, lacks a functional secretion signal, i.e. it lacks a secretion signal which allows for secretion of the polypeptide from the host cell into the fermentation broth. The terms “signal peptide”, “secretion sequence”, “secretion signal peptide” are used interchangeably herein.

Signal peptides are well-known in the art and can be found, in general, at the N-terminus of secreted Bacillus proteins, such as the AmyB, AmyE, AmyL AmyM, AmyQ, AmyS, AprE, AprH, AspB, BglC, BglS, Bpr, CelA, CelA, Csn, Epr, ForD, GGT, LacZ, LipA, LytB, LytD, Pel, PhoD, PhrK, Vpr, WapA, YbdN, YckD, YddT, YfhK, YfjS, YhfM, YjfA, YkwD, YncM, YnfF, YobB, YvcE, YvfO or YwbN polypeptide.

Exemplary Bacillus signal peptides as shown in the following Table A.

TABLE A
Exemplary signal peptides from various Bacillus polypeptides

| Species | Origin | SEQ ID NO | Sequence |
|---|---|---|---|
| Bacillus cereus | AmyB | 47 | MKNQFQYCCIVILSVVMLFVSLLIPQASSA |
| Bacillus subtilis | AmyE | 48 | MFAKRFKTSLLPLFAGFLLLFHLVLAG |
| Bacillus licheniformis | AmyL | 49 | MKQQKRLYARLLTLLFALIFLLPHSAAAA |
| Bacillus | AmyM | 50 | MKKKTLSLFVGLMLLIGLLFSGSLPYNPNAAEA |
| stearothermophilus | | | |
| Bacillus | AmyQ | 51 | MIQKRKRTVSFRLVLMCTLLFVSLPITKTSA |
| amyloliquefaciens | | | |
| Bacillus | AmyS | 52 | MLTFHRIIRKGWMFLLAFLLTALLFCPTGQPAKA |
| stearothermophilus | | | |
| Bacillus subtilis | AprE | 53 | MRSKKLWISLLFALTLIFTMAFSNMSAQA |
| Bacillus licheniformis | AprE | 54 | MMRKKSFWLGMLTAFMLVFTMASIASA |
| Bacillus pumilus | AprE | 55 | MKKKNVMTSVLLAVPLLFSAGFGGSMAQA |
| Bacillus clausii | AprH | 56 | MKKPLGKIVASTALLISVAFSSSIASA |
| Bacillus subtilis | AspB | 57 | MKLAKRVSALTPSTTLAITAKA |
| Bacillus subtilis | BglC | 58 | MKRSISIFITCLLITLLTMGGMIASPASA |
| Bacillus subtilis | BglS | 59 | MPYLKRVLLLLVTGLFMSLFAVTATASA |
| Bacillus subtilis | Bpr | 60 | MRKKTKNRLISSVLSTVVISSLLFPGAAGA |
| Streptomyces lividans | CelA | 61 | MGFGSAPIALCPLRTRRNALKRLLALLATGVSIV- |
| | | | GLTALAGPPAQA |
| Bacillus licheniformis | CelA | 62 | MMAEKVFSKNKIIGGKRMSYMKRSISVFIACFMVAVL- |
| | | | GISGIIAPKASA |
| Bacillus subtilis | Csn | 63 | MKISMQKADFWKKAAISLLVFTMFFTLMMSETVFA |
| Bacillus subtilis | Epr | 64 | MKNMSCKLVVSVTLFFSFLTIGPLAHA |
| Bacillus licheniformis | ForD | 65 | MKNHLYEKKKRKPLTRTIKATLAVLTMSIALVGGAT- |
| | | | VPSLA |
| Bacillus licheniformis | GGT | 66 | MRRLAFLVVAFCLAVGCFFSPVSKA |
| Bifidobacterium | LacZ | 67 | MAVRRLGGRIVAFAATVALSIPLGLLTNSAWA |
| bifidum | | | |
| Bacillus subtilis | LipA | 68 | MKFVKRRIIALVTILMLSVTSLFALQPSAKA |
| Bacillus subtilis | LytB | 69 | MKSCKQLIVCSLAAILLLIPSVSFA |
| Bacillus subtilis | LytD | 70 | MKKRLIAPMLLSAASLAFFAMSGSAQA |
| Bacillus subtilis | Pel | 71 | MKKVMLATALFLGLTPAGANA |
| Bacillus subtilis | PhoD | 72 | MAYDSRFDEWVQKLKEESFQNNTFDRRKFIQGAGKI- |
| | | | AGLSLGLTIAQSVGA |
| Bacillus subtilis | PhrK | 73 | MKKLVLCVSILAVILSGVA |
| Bacillus subtilis | Vpr | 74 | MKKGIIRFLLVSFVLFFALSTGITGVQAAPA |
| Bacillus subtilis | WapA | 75 | MKKRKRRNFKRFIAAFLVLALMISLVPADVLA |
| Bacillus subtilis | YbdN | 76 | MVKKWLIQFAVMLSVLSTFTYSASA |
| Bacillus subtilis | YckD | 77 | MKRITINIITMFIAAAVISLTGTAEA |
| Bacillus subtilis | YddT | 78 | MRKKRVITCVMAASLTLGSLLPAGYASA |
| Bacillus subtilis | YfhK | 79 | MKKKQVMLALTAAAGLGLTALHSAPAAKA |
| Bacillus subtilis | YfjS | 80 | MKWMCSICCAAVLLAGGAAQA |
| Bacillus subtilis | YhfM | 81 | MKKIVAAIVVIGLVFIAFFYLYSRSGDVYQSVDA |
| Bacillus subtilis | YjfA | 82 | MKRLFMKASLVLFAVVFVFAVKGAPAKA |
| Bacillus subtilis | YkwD | 83 | MKKAFILSAAAAVGLFTFGGVQQASA |
| Bacillus subtilis | YncM | 84 | MAKPLSKGGILVKKVLIAGAVGTAVLFGTLSSGIPGL- |
| | | | PAADA |
| Bacillus subtilis | YnfF | 85 | MIPRIKKTICVLLVCFTMLSVMLGPGATEVLA |
| Bacillus subtilis | YobB | 86 | MKIRKILLSSALSFGMLISAVPALA |
| Bacillus subtilis | YvcE | 87 | MRKSLITLGLASVIGTSSFLIPFTSKTASA |
| Bacillus licheniformis | YvfO | 88 | MKNVLAVFVVLIFVLGAFGTSGPAEA |
| Bacillus subtilis | YwbN | 89 | MSDEQKKPEQIHRRDILKWGAMAGAAVAIGAS- |
| | | | GLGGLAPLVASA |

As set forth above, preferably the polypeptide of interest lacks a functional signal peptide. Thus, the polypeptide of interest may comprise a signal peptide which is not functional, such a truncated signal peptide or a mutated signal peptide.

Whether a peptide acts as secretion signal peptide, or not, can be assessed by a bioinformatics approach using SignalP signal peptide prediction tool (Almagro Armenteros J J, Nielsen H.; SignalP 5.0 improves signal peptide predictions using deep neural networks. Nat Biotechnol. 2019 April; 37(4):420-423); or by measuring the secretion ability for a given polypeptide when fused to a potential secretion signal peptide as previously shown (Brockmeier U, Eggert T. Systematic screening of all signal peptides from Bacillus subtilis: a powerful strategy in optimizing heterologous protein secretion in Gram-positive bacteria. J Mol Biol. 2006 Sep. 22; 362(3):393-402).

The polypeptide of interest may be also a polypeptide that naturally comprises a signal peptide, wherein said signal peptide has been removed or inactivated. For example, it may be a AmyB, AmyE, AmyL AmyM, AmyQ, AmyS, AprE, AprH, AspB, BglC, BglS, Bpr, CelA, CelA, Csn, Epr, ForD, GGT, LacZ, LipA, LytB, LytD, Pel, PhoD, PhrK, Vpr, WapA, YbdN, YckD, YddT, YfhK, YfjS, YhfM, YjfA, YkwD, YncM, YnfF, YobB, YvcE, YvfO or YwbN polypeptide lacking a functional signal peptide.

### Promoter

Preferably, the at least one polynucleotide encoding a polypeptide of interest of interest is operably linked to a promoter that allows for expressing said polypeptide.

The term “operably linked” as used herein refers to a functional linkage between the promoter sequence and the polynucleotide encoding a polypeptide of interest, such that the promoter sequence is able to initiate transcription of the polynucleotide encoding a polypeptide of interest (herein also referred to as gene of interest).

A “promoter” or “promoter sequence” is a nucleotide sequence located upstream of a gene on the same strand as the gene that enables that gene's transcription. A promoter is followed by the transcription start site of the gene. A promoter is recognized by RNA polymerase (together with any required transcription factors), which initiates transcription. A functional fragment or functional variant of a promoter is a nucleotide sequence which is recognizable by RNA polymerase, and capable of initiating transcription.

An “active promoter fragment”, “active promoter variant”, “functional promoter fragment” or “functional promoter variant” describes a fragment or variant of the nucleotide sequences of a promoter, which still has promoter activity.

A promoter can be an “inducer-dependent promoter” or an “inducer-independent promoter” comprising constitutive promoters or promoters which are under the control of other cellular regulating factors.

The person skilled in the art is capable to select suitable promoters for expressing the third alanine racemase and the polypeptide of interest. For example, the polynucleotide encoding the polypeptide of interest is, preferably, operably linked to an “inducer-dependent promoter” or an “inducer-independent promoter”. Further, the polynucleotide encoding the third alanine racemase is, preferably, operably linked to an “inducer-independent promoter”, such as a constitutive promoter.

An “inducer dependent promoter” is understood herein as a promoter that is increased in its activity to enable transcription of the gene to which the promoter is operably linked upon addition of an “inducer molecule” to the fermentation medium. Thus, for an inducer-dependent promoter, the presence of the inducer molecule triggers via signal transduction an increase in expression of the gene operably linked to the promoter. The gene expression prior activation by the presence of the inducer molecule does not need to be absent, but can also be present at a low level of basal gene expression that is increased after addition of the inducer molecule. The “inducer molecule” is a molecule, the presence of which in the fermentation medium is capable of affecting an increase in expression of a gene by increasing the activity of an inducer-dependent promoter operably linked to the gene. Preferably, the inducer molecule is a carbohydrate or an analog thereof.

In one embodiment, the inducer molecule is a secondary carbon source of the Bacillus cell. In the presence of a mixture of carbohydrates, cells selectively take up the carbon source that provide them with the most energy and growth advantage (primary carbon source). Simultaneously, they repress the various functions involved in the catabolism and uptake of the less preferred carbon sources (secondary carbon source). Typically, a primary carbon source for Bacillus is glucose and various other sugars and sugar derivates being used by Bacillus as secondary carbon sources. Secondary carbon sources include e.g. mannose or lactose without being restricted to these.

Examples of inducer dependent promoters are given in the table below by reference to the respective operon:

| Operon | Regulator a) | Type b) | Inducer | Organism |
|---|---|---|---|---|
| sacPA | SacT | AT | sucrose | B. subtilis |
| sacB | SacY | AT | sucrose | B. subtilis |
| bgl PH | LicT | AT | β-glucosides | B. subtilis |
| licBCAH | LicR | A | oligo-β-gluco- | B. subtilis |
| | | | sides | |
| levDEFG sacL | LevR | A | fructose | B. subtilis |
| mtIAD | MtIR | A | mannitol | B. subtilis |
| manPA-yjdF | ManR | A | mannose | B. subtilis |
| manR | ManR | A | mannose | B. subtilis |
| bglFB bgIG | BgIG | AT | β-glucosides | E. coli |
| lacTEGF | LacT | AT | lactose | L. casei |
| lacZYA | lacl | R | Allolactose; | E. coli |
| | | | IPTG (Isopropyl | |
| | | | β-D-1-thiogalac- | |
| | | | topyranoside) | |
| araBAD | araC | AR | L-arabinose | E. coli |
| xylAB | XyIR | R | Xylose | B. subtilis |

a: transcriptional regulator protein
b: A: activator
AT: antiterminator
R: repressor
AR: activator/repressor

In contrast thereto, the activity of promoters that do not depend on the presence of an inducer molecule (herein called ‘inducer-independent promoters’) are either constitutively active or can be increased regardless of the presence of an inducer molecule that is added to the fermentation medium.

Constitutive promoters are independent of other cellular regulating factors and transcription initiation is dependent on sigma factor A (sigA). The sigA-dependent promoters comprise the sigma factor A specific recognition sites ‘−35’-region and ‘−10’-region.

Preferably, the ‘inducer-independent promoter’ sequence is selected from the group consisting of constitutive promoters not limited to promoters Pveg, PlepA, PserA, PymdA, Pfba and derivatives thereof with different strengths of gene expression (Guiziou et al, (2016): Nucleic Acids Res. 44(15), 7495-7508), the aprE promoter of Subtilisin encoding aprE gene of Bacilli, the bacteriophage SPO1 promoters P4, P5, P15 (WO15118126), the crylIIA promoter from Bacillus thuringiensis (WO9425612), the amyQ promoter from Bacillus amyloliquefaciens, the amyL promoter and promoter variants from Bacillus licheniformis (U.S. Pat. No. 5,698,415) and combinations thereof, or active fragments or variants thereof, preferably an aprE promoter sequence.

WO9102792 discloses the functionality of the promoter of the alkaline protease gene for the largescale production of subtilisin Carlsberg-type protease in Bacillus licheniformis and its production in a fermentation process.

The term “transcription start site” or “transcriptional start site” shall be understood as the location where the transcription starts at the 5′ end of a gene sequence. In prokaryotes the first nucleotide, referred to as +1 is in general an adenosine (A) or guanosine (G) nucleotide. In this context, the terms “sites” and “signal” can be used interchangeably herein.

The term “expression” or “gene expression” means the transcription of a specific gene or specific genes or specific nucleic acid construct. The term “expression” or “gene expression” in particular means the transcription of a gene or genes or genetic construct into structural RNA (e.g., rRNA, tRNA) or mRNA with or without subsequent translation of the latter into a protein. The process includes transcription of DNA and processing of the resulting mRNA product.

Further optionally the promoter comprises a 5′UTR. This is a transcribed but not translated region downstream of the −1 promoter position. Such untranslated region for example should contain a ribosome binding site to facilitate translation in those cases where the target gene codes for a peptide or polypeptide.

The promoter may be combined, for example, with a 5′UTR comprising one or more stabilizing elements. This way the mRNAs synthesized from the promoter region may be processed to generate mRNA transcript with a stabilizer sequence at the 5′ end of the transcript. Preferably such a stabilizer sequence at the 5′ end of the mRNA transcripts increases their half-life as described by Hue et al, 1995, Journal of Bacteriology 177: 3465-3471. Suitable mRNA stabilizing elements are those described in
WO08148575, preferably SEQ ID NO. 1 to 5 of WO08140615, or fragments of these sequences which maintain the mRNA stabilizing function, and in
WO08140615, preferably Bacillus thuringiensis Cry/l/A mRNA stabilising sequence or bacteriophage SP82 mRNA stabilising sequence, more preferably a mRNA stabilising sequence according to SEQ ID NO. 4 or 5 of WO08140615, more preferably a modified mRNA stabilising sequence according to SEQ ID NO. 6 of WO08140615, or fragments of these sequences which maintain the mRNA stabilizing function.

Preferred mRNA stabilizing elements are selected from the group consisting of aprE, grpE, cotG, SP82, RSBgsiB, Cry/l/A mRNA stabilizing elements, or according to fragments of these sequences which maintain the mRNA stabilizing function. A preferred mRNA stabilizing element is the grpE mRNA stabilizing element (corresponding to SEQ ID NO. 2 of WO08148575).

The 5′UTR also preferably comprises a modified rib leader sequence located downstream of the promoter and upstream of a ribosome binding site (RBS). In the context of the present invention a rib leader is herewith defined as the leader sequence upstream of the riboflavin biosynthetic genes (rib operon) in a Bacillus cell, more preferably in a Bacillus subtilis cell. In Bacillus subtilis, the rib operon, comprising the genes involved in riboflavin biosynthesis, include ribG (ribD), ribB (ribE), ribA, and ribH genes. Transcription of the riboflavin operon from the rib promoter (Prib) in B. subtilis is controlled by a riboswitch involving an untranslated regulatory leader region (the rib leader) of almost 300 nucleotides located in the 5′-region of the rib operon between the transcription start and the translation start codon of the first gene in the operon, ribG. Suitable rib leader sequences are described in WO2015/1181296, in particular pages 23-25, incorporated herein by reference.

### IspA Knock-Out

In an embodiment of the present invention, the ispA gene has been inactivated in the Bacillus host cell. The intracellular serine protease gene ispA is encoding for the intracellular serine protease IspA. Serine proteases (EC 3.4.21) are enzymes that cleave peptide bonds in proteins, in which serine serves as the nucleophilic amino acid at the active site of enzyme.

The term “inactivated” in connection with the ispA gene, preferably, means that the enzymatic activity of the intracellular serine protease IspA encoded by said gene has been reduced as compared to the intracellular serine protease IspA activity in a control cell. A control cell is a corresponding host cell in which the ispA gene has not been inactivated, i.e. a corresponding host cell which comprises said chromosomal ispA gene. Preferably, the enzymatic activity of the intracellular serine protease in the bacterial host cell of the present invention has been reduced by at least 40% such as at least 50%, at least 60%, at least 70%, at least 80%, or at least 90% as compared to the corresponding enzymatic activity of IspA in the control cell. More preferably, said enzymatic activity has been reduced by at least 95%. Most preferably, said enzymatic activity has been reduced by 100%, i.e. has been eliminated completely.

The inactivation of the ispA gene as referred to herein may be achieved by any method deemed appropriate. In an embodiment, the ispA gene encoding the intracellular serine protease IspA has been inactivated by mutation, i.e. by mutating said gene. Preferably, said mutation is a deletion, i.e. said chromosomal ispA gene has been deleted.

As used herein, the “deletion” of a gene refers to the deletion of the entire coding sequence, deletion of part of the coding sequence, or deletion of the coding sequence including flanking regions. The end result is that the deleted gene is effectively non-functional. In simple terms, a “deletion” is defined as a change in either nucleotide or amino acid sequence in which one or more nucleotides or amino acid residues, respectively, have been removed (i.e., are absent). Thus, a deletion strain has fewer nucleotides or amino acids than the respective wild-type organism.

In an embodiment, the ispA gene has been inactivated by gene silencing. Gene silencing can be achieved by introducing into said bacterial host cell antisense expression constructs that result in antisense RNAs complementary to the mRNA of said chromosomal gene, thereby inhibiting expression of said gene. Alternatively, the expression of said gene can be inhibited by blocking transcriptional initiation or transcriptional elongation through the mechanism of CRISPR-inhibition (WO18009520).

The term “inactivating a protein” means that the protein is altered in its amino acid sequence in a way that the function of the protein in the cell has been reduced as compared to the non-altered protein. Preferably, the function of the protein in the bacterial host cell of the present invention has been reduced by at least 10%, such as at least 40%, as at least 50%, at least 60%, at least 70%, at least 80%, or at least 90% as compared to the corresponding function of the non-altered protein. More preferably, said function has been reduced by at least 95%. Most preferably, the function has been reduced by 100%, i.e. the protein is completely non-functional.

A “control cell” as referred to herein is a control cell of the same species which does not carry the respective modification, preferably which differs from the host cell only in that it does not carry the respective modification. Thus, the control cell is an unmodified cell, such as a wild-type cell, i.e. an unmodified wild-type cell. Thus, the control cell may be a Bacillus cell in which the ispA gene is not inactivated.

The sequences of the ispA gene and the IspA polypeptide are well-known for many Bacillus species.

The coding sequence of the Bacillus licheniformis ispA gene is shown in SEQ ID NO: 25. The IspA polypeptide encoded by said gene has an amino acid sequence as shown in SEQ ID NO: 121.

The coding sequence of the Bacillus subtilis ispA gene is shown in SEQ ID NO: 102. The IspA polypeptide encoded by said gene has an amino acid sequence as shown in SEQ ID NO: 119.

The amino acid sequence of the Bacillus pumilus IspA polypeptide is shown in SEQ ID NO: 124.

Moreover, the sequences for Bacillus IspA polypeptides are shown in the following table B.

TABLE B
Overview of IspA polypeptides of
various Bacillus species

| | SEQ ID NO: |
|---|---|
| | of the |
| | amino acid |
| | sequence |
| | of the ispA |
| Species | polypeptide |
| B. licheniformis | 121 |
| B. subtilis | 119 |
| Bacillus paralicheniformis | 123 |
| Bacillus pumilus | 124 |
| Bacillus megaterium | 125 |
| Bacillus thuringiensis | 126 |
| Bacillus methanolicus | 127 |
| MGA3 | |
| Bacillus velezensis | 128 |
| FZB42 | |
| Bacillus amyloliquefaciens | 129 |
| XH7 | |
| Bacillus cereus | 130 |
| Bacillus mojavensis | 131 |

For industrial fermentation processes, the bacterial host cell may be genetically modified to meet the needs of highest product purity and regulatory requirements. It is therefore in the scope of the invention to use Bacillus production hosts that may additionally contain modifications, e.g., deletions or disruptions, of other genes that may be detrimental to the production, recovery or application of a polypeptide of interest. In one embodiment, a bacterial host cell is a protease-deficient cell. The bacterial host cell, e.g., Bacillus cell, preferably comprises a disruption or deletion of extracellular protease genes including but not limited to aprE, mpr, bpr, vpr, epr, nprE, nprB and/or wprA, such as aprE, mpr, bpr, vpr, epr and/or wprA. Thus, one or more extracellular proteases selected from aprE, mpr, bpr, vpr, epr, nprE, nprB and wprA may be knocked-out, i.e. inactivated, in the host cell.

In a preferred embodiment the bacterial host cell further comprises a disruption or deletion of extracellular protease genes aprE, mpr. In a more preferred embodiment the bacterial host cell further comprises a disruption or deletion of extracellular protease genes aprE, mpr, bpr, vpr.

Further, the intracellular protease aprX may be inactivated.

The coding sequence of the Bacillus licheniformis aprX gene is shown in SEQ ID NO: 104. The AprX polypeptide encoded by said gene has an amino acid sequence as shown in SEQ ID NO: 122.

The coding sequence of the Bacillus subtilis aprX gene is shown in SEQ ID NO: 103. The AprX polypeptide encoded by said gene has an amino acid sequence as shown in SEQ ID NO: 120.

Moreover, one or more of the enzymes amylase, amyloglucosidase, cellulase, chitinase, lipase, phospholipase, esterase, invertase, arylsulfatase, mannanase, pectinases and pectate lyase may be inactivated.

Further, preferably the bacterial host cell does not produce spores. Further, preferably the bacterial host cell, e.g., a Bacillus cell, comprises a disruption or deletion of genes involved in sporulation. Genes involved in sporulation are well known in the art (EP1391502), comprising but not limited to sigE, sigF, spolIGA, spollE, sigG, spolVCB, yqfD. In a preferred embodiment, the sigF gene is deleted. Further, preferably the bacterial host cell, e.g., Bacillus cell, comprises a disruption or deletion of one of the genes involved in the biosynthesis of surfactin, e.g., srfA, srfB, srfC, and/or srfD, see, for example, U.S. Pat. No. 5,958,728. It is also preferred that the bacterial host cell comprises a disruption or deletion of one of the genes involved in the biosynthesis of polyglutamic acid (US2016002591). Accordingly, at least one gene involved in poly-gamma-glutamate (pga) production has been inactivated (such as deleted). Preferably, the at least one gene involved in poly-gamma-glutamate (pga) is at least one gene selected from ywsC (pgsB), ywtA (pgsC), ywtB (pgsA) and ywtC (pgsE). Preferably, all aforementioned genes, i.e. ywsC (pgsB), ywtA (pgsC), ywtB (pgsA) and ywtC (pgsE) have been inactivated (such as deleted). Other genes, including but not limited to the amyE gene, which are detrimental to the production, recovery or application of a polypeptide of interest may also be disrupted or deleted.

In one embodiment, the Bacillus cell comprises a selectable marker. The selectable marker can be antibiotic resistance markers such as ampicillin, kanamycin, erythromycin, chloramphenicol or tetracycline, or an auxotrophic resistance marker.

Methods for the generation of modified Bacillus cells and altered proteins, e.g., by introduction of foreign nucleic acids, chromosomal gene deletion, substitution, mutation, and inactivation, are known in the art.

The introduction of DNA into a host cell, in one embodiment a Bacillus cell, may, for instance, be effected by protoplast transformation (see, e.g., Chang and Cohen, 1979, Molecular General Genetics 168: 111-115), by using competent cells (see, e.g., Young and Spizizen, 1961, Journal of Bacteriology 81: 823-829, or Dubnau and Davidoff-Abelson, 1971, Journal of Molecular Biology 56: 209-221), by electroporation (see, e.g., Shigekawa and Dower, 1988, Biotechniques 6: 742-751), or by conjugation (see, e.g., Koehler and Thorne, 1987, Journal of Bacteriology 169: 5271-5278). Specific transformation protocols are known in the art for various types of host cells (see, e.g., for E. coli protoplast transformation Hanahan, 1983, J. Mol. Biol. 166: 557-580).

Gene inactivation can be achieved by homologous recombination, i.e. an incoming DNA molecule comprises sequences that are homologous to the 5′ and 3′ flanking sequences of the target sequence on the chromosome of the host cell (e.g. Bacillus) to be inactivated. Subsequently the sequence between said flanking sequences is replaced by the homologous sequences of the incoming DNA molecule in the process of homologous recombination, i.e. the sequence is deleted from the chromosome. Likewise “gene integration”, i.e. a DNA sequence such as a gene expression cassette with or without a selectable marker, can be integrated into the chromosome of the bacterial host cell by homologous recombination. Hence, the DNA sequence to be integrated is flanked by DNA sequences that are homologous to the 5′ and 3′ flanking sequences on the chromosome. It is understood in terms of the invention that gene integration can also combine gene integration and gene deletion in one step, i.e. a DNA sequence on the chromosome is replaced by the incoming DNA sequence for gene integration.

Homologous recombination can be achieved by two different methods known in the art: By two consecutive rounds of homologous recombination (Campbell recombination) with circular plasmid DNA, e.g. based on the well-known temperature sensitive plasmid pE194 (Nahrstedt et al., Strain development in Bacillus licheniformis: construction of biologically contained mutants deficient in sporulation and DNA repair. J Biotechnol. 2005 Sep. 29; 119(3):245-54).

The integration of the deletion plasmid containing an incoming DNA molecule comprising sequences that are homologous to the 5′ and 3′ flanking sequences of the target sequence on the chromosome is achieved by a first homologous recombination (Campbell recombination) with the first homologous region under selective conditions for the selectable marker and cultivation at the non-permissive temperature, i.e. that blocks plasmid replication. The second homologous recombination with the second homologous region is achieved by removal of selective pressure and cultivation at the permissive temperature, i.e. plasmid replication takes place, resulting in excision of the plasmid from the chromosome.

Alternatively, a non-replicative ‘suicide’ plasmid can be used forcing the integration by selection on the selectable marker. Only cells that have integrated the plasmid into the genome by homologous recombination are able to grow under the selective conditions. Plasmid removal/excision from the chromosome is achieved with a second homologous recombination which is forced by the activation of a counterselection marker present on the plasmid.

The second method of homologous recombination refers to two homologous recombination events simultaneously taking place, also known as ‘double crossing over’ or ‘double homologous recombination. The incoming DNA sequence is linear and can be obtained by PCR, linearization of plasmid DNA or preparation of chromosomal DNA which inevitable results in fragmented linear DNA. WO0308125 uses linear DNA constructs (either linearized plasmids or PCR fragments) comprising a selectable marker flanked by the 5′ and 3′ homologous regions which are used for genomic integration via double crossing over homologous recombination. It is well understood that next to the selectable marker additional DNA, such as gene expression cassettes, when flanked by said homologous region are integrated into the chromosome of the bacterial host cell.

Homologous recombination requires DNA sequences homologous to the 5′ and 3′ flanking sequences of the target sequence on the chromosome of the host cell of sufficient size, hence should contain a sufficient number of nucleic acid such as 100 to 1,500 base pairs, preferably 400 to 1,500 base pairs, and most preferably 800 to 1,500 base pairs, which have a high degree of identity to the corresponding target sequence to enhance the probability of homologous recombination (Dubnau, 1993, Genetic exchange and homologous recombination. In Bacillus subtilis and Other Gram-positive Bacteria, p. 555-584. Edited by A. I. Sonenshein, J. A. Hoch & R. Losick, Washington DC, American Society for Microbiology; Michel and Ehrlich, 1984, The EMBO Journal, vol. 3, pp. 2879-2884).

Gene inactivation by deletion/insertion/substitution can also be achieved by CRISPR/Cas9 genome editing technologies where the CRISPR cutting properties could be used to disrupt genes in almost any organism's genome with unprecedented ease (Mali P, et al (2013) Science. 339(6121):819-823; Cong L, et al (2013) Science 339(6121)). Recently it became clear that providing a template for repair, e.g. homologous regions, allowed for editing the genome with nearly any desired sequence at nearly any site, transforming CRISPR into a powerful gene editing tool (WO2014/150624, WO2014/204728).

CRISPR-based genome editing systems for application in gram positive organisms have been well described such as the Bacillus species based single-plasmid system approach, i.e. comprising the Cas9 endonuclease, the gRNA (e.g. sgRNA or crRNA/tracrRNA), repair homology sequences (donor DNA) on one single E. coli-Bacillus shuttle vector (Altenbuchner, (2016): Applied and environmental microbiology 82 (17), 5421-5427; Zhou, et al. (2019): International journal of biological macromolecules 122, 329-337), or dual plasmid system or with Cas9 endonuclease integrated into the Bacillus genome as described e.g. in WO2020/206202 and WO2020/206197.

Alternatively to “directed” methods of inactivation it is understood in the scope of the invention that whole-cell mutagenesis by applying mutagenizing conditions such as exposure of the cells to UV radiation, or chemical mutagenizing chemicals such as NTG (N-methyl-N′-nitro-N-nitrosoguanidine), EMS (ethyl-methane-sulfonate), in combination with screening and/or selection of the desired property, e.g. reduced lipase/esterase activity is a well-known approach to achieve functional inactivation.

Further, a gene may have been inactivated by gene silencing. Gene silencing can be achieved by introducing into said bacterial host cell antisense expression constructs that result in antisense RNAs complementary to the mRNA of the gene, thereby inhibiting expression of said genes. Alternatively, the expression of said gene can be inhibited by blocking transcriptional initiation or transcriptional elongation through the mechanism of CRISPR-inhibition (WO18/009520).

### Cultivation Step

In step b) of the method of the present invention, the Bacillus host cell is preferably cultivated under conditions which allow for intracellular accumulation of said at least one polypeptide of interest. In this step, the at least one polypeptide of interest is produced. The cultivations under conditions which allow for intracellular accumulation of said at least one polypeptide of interest is preferably achieved by expressing said at least one polypeptide of interest without a functional secretion signal peptide (as described herein above). Thus, the protein of interest produced by the Bacillus host cell is confined to the intracellular milieu of the host cell.

The term “cultivating” as used herein refers to keeping alive and/or propagating the modified host cell comprised in a culture at least for a predetermined time. The term encompasses phases of exponential cell growth at the beginning of growth after inoculation as well as phases of stationary growth. The cultivation conditions shall allow for the expression, i.e. the production, of the polypeptide of interest. Such conditions can be chosen by the skilled person without further ado. Further, the conditions are described herein below.

The invention provides methods for producing a protein of interest in a modified Bacillus cell by culturing the modified cell that is capable of producing a protein of interest and growing the cell under suitable growth conditions for expressing the protein of interest. In some embodiments, the host cells and modified host cells of the present invention are cultured in conventional nutrient media. The suitable specific culture conditions, such as temperature, pH and the like are known to those skilled in the art.

Additional preferred culture media are chemically defined fermentation media that are well known to those of skill in the art and are described in various reference publications (e.g. WO 2020/169563 A1). Exemplary conditions for the cultivation of the modified host cell are described in the Examples section. In an embodiment of the method of the present invention, the cultivation step is carried out as fed batch cultivation, preferably in a chemically defined fermentation medium, preferably, in a fermenter. In an embodiment, the fermenter comprises at least 1 m³, at least 10 m³, at least 50 m³ fermentation broth.

An industrially relevant fermentation process preferably encompasses a fermentation process on a volume scale which is at least 1 m³ with regard to the nominal fermenter size, preferably at least 5 m³, more preferably at least 10 m³, even more preferably at least 25 m³, most preferably at least 50 m³. Preferably, the industrially relevant fermentation process encompasses a fermentation process on a volume scale which is 1-500 m³ with regard to the nominal fermenter size, preferably 5-500 m³, more preferably 10-500 m³, even more preferably 25-500 m³, most preferably 50-500 m³.

Preferably, the temperature of the fermentation broth during cultivation is 25° C. to 45° C., preferably, 27° C. to 40° C., more preferably, 27° C. to 37° C.

Preferably, oxygen is added to the fermentation medium during cultivation, preferably by agitation and gassing, preferably with 0-3 bar air or oxygen. Oxygen is usually provided during the cultivation of the cells by aeration of the fermentation media by stirring or gassing. However, hydrogen and oxygen are also contained within the chemically defined carbon and/or chemically defined nitrogen source (see below) and can be provided that way.

Preferably, the fermentation time is preferably 1-200 hours, more preferably, 1-120 hours, most preferably 10-90h.

Preferably, a carbon source and/or nitrogen source is added to the fermentation broth during cultivation. Preferably, the cultivation is carried out in the chemically defined fermentation medium as the amount of added carbon source and nitrogen source can be precisely controlled.

Preferably, the chemically defined carbon source is selected from the group consisting of carbohydrates, organic acids, hydrocarbons, and alcohols and mixtures thereof. Preferred carbohydrates are selected from the group consisting of glucose, fructose, galactose, xylose, arabinose, sucrose, maltose, maltotriose, lactose, dextrin, maltodextrins, starch and inulin, and mixtures thereof. In particular, the carbon source is glucose. Preferred alcohols are selected from the group consisting of glycerol, methanol and ethanol, inositol, mannitol and sorbitol and mixtures thereof. Preferred organic acids are selected from the group consisting of acetic acid, propionic acid, lactic acid, formic acid, malic acid, citric acid, fumaric acid and higher alkanoic acids and mixtures thereof. Preferably, the chemically defined carbon source comprises glucose or sucrose. More preferably, the chemically defined carbon source comprises glucose, preferably wherein the predominant amount of the chemically defined carbon source is provided as glucose. Most preferably, the chemically defined carbon source is glucose. It is to be understood that the defined chemically defined carbon source can be provided in form of a syrup, preferably as glucose syrup. As understood herein, the term “glucose” shall include glucose syrups. A glucose syrup is a viscous sugar solution with high sugar concentration. The sugars in glucose syrup are mainly glucose and to a minor extent also maltose and maltotriose in varying concentrations depending on the quality grade of the syrup. Preferably, besides glucose, maltose and maltotriose the syrup can comprise up to 10%, preferably, up to 5%, more preferably up to 3% impurities. Preferably, the syrup is corn syrup.

The chemically defined nitrogen source is preferably selected from the group consisting of urea, ammonia, nitrate, nitrate salts, nitrite, ammonium salts such as ammonium chloride, ammonium sulphate, ammonium acetate, ammonium phosphate and ammonium nitrate, and amino acids such as glutamate or lysine and combinations thereof. More preferably, a chemically defined nitrogen source is selected from the group consisting of ammonia, ammonium sulphate and ammonium phosphate. Most preferably, the chemically defined nitrogen source is ammonia. The use of ammonia as a chemically defined nitrogen source has the advantage that ammonia can additionally function as a pH controlling agent. Preferably, at least 0.1 g of nitrogen is added per liter of initial fermentation medium in the initial fermentation medium.

### Lysis of Cells

After step b), the produced polypeptide of interest may be recovered from the cells. This is achieved by cell lysis. As described herein above, the product is preferably recovered by mild lysis conditions. This is achieved by stop of aeration and stop of addition of feed (step c)) and by incubating the cells for at least five hours, preferably, at an increased temperature (step d)).

The stop of aeration is preferably achieved by stopping the oxygen supply. Specifically, the stop of aeration is the stop of the supply of oxygen to the fermentation broth by active aeration.

The stop of addition of feed is preferably achieved by stopping the addition of the carbon source and/or nitrogen source, i.e. the carbon source and/or nitrogen source that was added during the cultivation step. More preferably, the stop of addition of feed is preferably achieved by stopping the addition of the carbon source, i.e. the carbon source that was added during the cultivation step. Thus, a carbon source is preferably no longer added to the fermentation broth.

In step d) of the method of the present invention, the cells are incubated (i.e. stored) for a period of at least five hours, such as at least 8 hours, such as 12 hours, such as 12 to 36 hours, such as 12 hours. Further, the cells are incubated for a period of 5-40 hours. Preferably, the cells are kept in the fermentation broth for incubation in step b). Thus, the cells are not separated from the fermentation broth prior to the incubation in step d). Further, the fermentation broth with the Bacillus cells may remain in the fermenter. During the incubation in step d), the cultivation broth comprising the cells may be agitated or stirred. Preferably, the cells are stirred.

In an embodiment, the cells are incubated in step d) at an increased temperature (at least for a certain period). In accordance with the present invention, an increased temperature is preferably a temperature of about 40° C. to about 60° C., more preferably of about 42° C. to about 57° C., even more preferably, of about 45° C. to about 55° C., and most preferably of about 50° C.

The cells are preferably incubated at an increased temperature for a certain period, for example for at least five hours, such as at least 10 hours. Preferably, the cells are stored (i.e. incubated) for 5-40 hours, more preferably for 5 to 30 hours, even more preferably for 10 to 30 hours, and most preferably for 10 to 24 hours.

In an embodiment, step d), thus, comprises:
d1) incubating the cells at a temperature of 40° C. to about 60° C., more preferably of about 42° C. to about 57° C., even more preferably, of about 45° C. to about 55° C., and most preferably of about 50° C. for at least 5 hours, such as at least 10 hours. Preferably, the cells are incubated for 5 to 40 hours, more preferably for 5 to 30 hours, even more preferably for 10 to 30 hours, and most preferably for 10 to 24 hours.

In an alternative embodiment, the cells are incubated at a low temperature prior to incubating them at an increased temperature. In accordance with the present invention, a low temperature is preferably a temperature of about 20° C. to about 40° C., more preferably of about 20° C. to about 35° C., even more preferably, of about 25° C. to about 35° C., and most preferably of about 30° C. Preferably, the cells are incubated at a low temperature for a certain period, for example for at least 5 hours, more preferably for at least 24 hours, most preferably at least 30 hours, or most preferably at least 36 hours. Preferably, the cells are incubated for a period of 5-48 hours, more preferably for 20 to 48 hours, even more preferably for 24 hours to 48 hours, and most preferably for 30 to 40 hours.

In an embodiment, step d), thus, comprises:
d2) incubating the cells at a temperature of about 20° C. to about 40° C., more preferably of about 20° C. to about 35° C., even more preferably, of about 25° C. to about 35° C., and most preferably of about 30° C. for at least 5 hours, in particular for at least 24 hours. Preferably, the cells are incubated for a period of 5-48 hours, more preferably for 20 to 48 hours, even more preferably for 24 hours to 48 hours, and most preferably for 30 to 40 hours.

After incubating the cells at a low temperature as described above, the cell may be also incubated at an increased temperature, for example for at least 2 hours, or at least 5 hours.

Preferably, the temperature in step d) is actively controlled. Thus, the temperature is kept constant during the incubation (e.g. by heating and/or cooling). Active control of the temperature can be achieved by a temperature control system.

Preferably, the cells are subjected to low mild lysis conditions. Thus, the lysis is not achieved by the addition of additives, preferably not by enzymatic lysis, such as lysis caused by adding lysozyme to the fermentation broth, and not by adding detergents, and not by mechanical lysis, such as ultrasound or French press. Further, the lysis is, preferably, not achieved by the change in pH to either pH<3 or >10 or salt concentration and not by hypoosmotic shock.

By carrying out steps c) and steps d), the cells comprised by the fermentation broth are lysed by stopping addition of feed and aeration and incubating the cells as set forth above. Preferably, at least 70%, such as at least 80%, such as at least 90%, or 100% of cells are lysed after step d) such as after step d1) or d2). How to determine the proportion of lysed cells is, e.g., described in the Examples section.

The method of the present invention may comprise further steps after step d, such as the isolation of the at least one protein of interest from the fermentation broth by separation of lysed biomass by addition of e.g. polyanionic or polycationic polymer flocculants and application of e.g. filter press or microfiltration generating a cleared fermentation supernatant. Further, the isolation of the at least one protein of interest, and/or removal of undesired contaminants such as but not limited to host cell proteins, nucleic acids, pigments is achieved by ultrafiltration, application of adsorbents, chromatographic separation (HIC, IEX) from the cleared fermentation supernatant. The at least one protein of interest may be recovered by methods known in the art. In some embodiments, the protein of interest produced by the cells is recovered by conventional procedures, including, but not limited to separating the host cell debris from the medium by centrifugation or filtration, precipitating the proteinaceous components of the supernatant or filtrate by means of a salt (e.g., ammonium sulfate) or chromatographic purification (e.g., ion exchange, gel filtration, affinity, etc.). Thus, any method suitable for recovering the protein of interest finds use in the present invention. Indeed, it is not intended that the present invention is limited to any particular purification method.

In an embodiment, the method of the present invention comprises the step of purifying the polypeptide of interest. Preferably, a solution (such as a concentrated solution) is provided after step d) comprising at least 10 g/l, more preferably at least 30 g/l, and most preferably at least 50 g/l of the polypeptide of interest. Preferably, the purity of the at least one polypeptide of interest in the provided solution is at least 40%, more preferably, at least 50%, and most preferably, at least 70%. The purity can be determined by calculating the amount of the polypeptide of interest as percentage of the total protein content. The total protein content can be determined using protein quantitation assays such the Bradford assay or BCA method. The amount of the polypeptide of interest can be determined by measuring the enzymatic activity with the known specific enzymatic activity (U/mg) of the polypeptide of interest. Alternatively, the amount of the polypeptide of interest can be quantified using SDS-PAGE and Coomassie Blue staining using reference quantitation proteins or a defined amount of the polypeptide of interest. Alternatively, the total amount of proteins and the polypeptide of interest can be quantified by SDS PAGE and Coomassie Blue staining using an image software such as the Software Image Lab™ Version 6.0.0 build 2.

The explanations and definitions given herein above in connection with the method of the present invention apply mutatis mutandis to the following:

The present invention further relates to a Bacillus host cell comprising at least one polynucleotide encoding a polypeptide of interest lacking a functional secretion sequence, wherein the ispA gene has been inactivated in the Bacillus host cell.

The skilled person understands the expression that ispA gene has been inactivated in the host cell. Thus, the unmodified wild-type cell, preferably comprises an ispA gene. Preferably, the gene has been actively inactivated by methods described above.

The present invention also relates to a method of producing a Bacillus host cell of the present invention. Preferably, the modified Bacillus host cell is obtained or is obtainable by the steps of
a) providing a Bacillus cell comprising an ispA gene,
b) inactivating the ispA gene, and
c) introducing at least one polynucleotide encoding at least one polypeptide of interest into the cell.

The present invention also relates to a method for producing a food product, the method comprising steps of producing at least one polypeptide of interest by carrying out the steps a), b), c) d) of the method of the present invention for producing a polypeptide of interest, wherein said at least one polypeptide of interest is an enzyme involved in food production, and contacting the polypeptide of interest with the food product.

Optionally, the above method comprises the step of isolating the polypeptide of interest from said fermentation broth after carrying out step d.

In an embodiment, the food product is a dairy product comprising galactooligosaccharides and the polypeptide of interest is a ß-Galactosidase (EC 3.2.1.23).

The present invention, thus, relates to a method for producing a dairy product comprising galactooligosaccharides, the method comprising steps of producing a polypeptide of interest by carrying out the steps a), b), c) d) of the method of the present invention for producing a polypeptide of interest, wherein said polypeptide of interest is a ß-Galactosidase, and contacting the polypeptide of interest with a dairy product.

### EXAMPLES

### Materials and Methods

The following examples only serve to illustrate the invention. The numerous possible variations that are obvious to a person skilled in the art also fall within the scope of the invention.

Unless otherwise stated the following experiments have been performed by applying standard equipment, methods, chemicals, and biochemicals as used in genetic engineering and fermentative production of chemical compounds by cultivation of microorganisms.

Electrocompetent Bacillus licheniformis Cells and Electroporation

Transformation of DNA into a Bacillus licheniformis strain as described in U.S. Pat. No. 5,352,604 is performed via electroporation. Preparation of electrocompetent Bacillus licheniformis cells and transformation of DNA is performed as essentially described by Brigidi et al (Brigidi, P., Mateuzzi, D. (1991). Biotechnol. Techniques 5, 5) with the following modification: Upon transformation of DNA, cells are recovered in 1 ml LBSPG buffer and incubated for 60 min at 37° C. (Vehmaanpers J., 1989, FEMS Microbio. Lett., 61: 165-170) following plating on selective LB-agar plates. In order to overcome the Bacillus licheniformis specific restriction modification system of Bacillus licheniformis strains, plasmid DNA is isolated from Ec #098 cells as described below.

### Plasmid Isolation

Plasmid DNA was isolated from Bacillus and E. coli cells by standard molecular biology methods or the alkaline lysis method (Birnboim, H. C., Doly, J. (1979). Nucleic Acids Res 7(6): 1513-1523). Bacillus cells were in comparison to E. coli treated with 10 mg/ml lysozyme for 30 min at 37° C. prior to cell lysis.

### Molecular Biology Methods and Techniques

Standard methods in molecular biology not limited to cultivation of Bacillus and E. coli microorganisms, electroporation of DNA, isolation of genomic and plasmid DNA, PCR reactions, cloning technologies were performed by well established method.

### Strains

B. subtilis Strain Bs #056

The prototrophic Bacillus subtilis strain KO-7S (BGSCID:1S145; Zeigler D. R) was made competent according to the method of Spizizen (Anagnostopoulos, C. and Spizizen, J. (1961). J. Bacteriol. 81, 741-746.) and transformed with the linearized DNA-methyltransferase expression plasmid pMIS012 for integration of the DNA-methyltransferase into the amyE gene as described for the generation of B. subtilis Bs #053 in WO2019016051. Cells were spread and incubated overnight at 37° C. on LB-agar plates containing 10 μg/ml chloramphenicol. Grown colonies were picked and stroked on both LB-agar plates containing 10 μg/ml chloramphenicol and LB-agar plates containing 10 μg/ml chloramphenicol and 0.5% soluble starch (Sigma) following incubation overnight at 37° C. The starch plates were covered with iodine containing Lugols solution and positive integration clones identified with negative amylase activity. Genomic DNA of positive clones was isolated by standard phenol/chloroform extraction methods after 30 min treatment with lysozyme (10 mg/ml) at 37° C., following analysis of correct integration of the MTase expression cassette by PCR. The resulting B. subtilis strain is named Bs #056.

E. coli Strain Ec #098

E. coli strain Ec #098 is an E. coli INV110 strain (Life technologies) carrying the DNA-methyltransferase encoding expression plasmid pMDS003 WO2019/016051.

Generation of Bacillus licheniformis Gene k.o Strains

For gene deletion in a Bacillus licheniformis strain as described in U.S. Pat. No. 5,352,604 and derivatives thereof deletion plasmids were transformed into E. coli strain Ec #098 made competent according to the method of Chung (Chung, C. T., Niemela, S. L., and Miller, R. H. (1989). One-step preparation of competent Escherichia coli: transformation and storage of bacterial cells in the same solution. Proc. Natl. Acad. Sci. U.S.A 86, 2172-2175), following selection on LB-agar plates containing 100 μg/ml ampicillin and 30 μg/ml chloramphenicol at 37° C. Plasmid DNA was isolated from individual clones and analyzed for correctness by PCR analysis. The isolated plasmid DNA carries the DNA methylation pattern of Bacillus licheniformis as described in WO2019/016051 and is protected from degradation upon transfer into Bacillus licheniformis.

aprE Gene Deletion Strain Bli #002

Electrocompetent Bacillus licheniformis cells as described in U.S. Pat. No. 5,352,604 were prepared as described above and transformed with 1 μg of pDe1003 aprE gene deletion plasmid isolated from E. coli Ec #098 following plating on LB-agar plates containing 5 μg/ml erythromycin at 30° C.

The gene deletion procedure was performed as described in the following:

Plasmid carrying Bacillus licheniformis cells were grown on LB-agar plates with 5 μg/ml erythromycin at 45° C. forcing integration of the deletion plasmid via Campbell recombination into the chromosome with one of the homology regions of pDe1003 homologous to the sequences 5′ or 3′ of the aprE gene. Clones were picked and cultivated in LB-media without selection pressure at 45° C. for 6 hours, following plating on LB-agar plates with 5 μg/ml erythromycin at 30° C. Individual clones were picked and analyzed by colony-PCR with oligonucleotides SEQ ID 06 and SEQ ID 07 for successful deletion of the aprE gene. Putative deletion positive individual clones were picked and taken through two consecutive overnight incubation in LB media without antibiotics at 45° C. to cure the plasmid and plated on LB-agar plates for overnight incubation at 30° C. Single clones were again restreaked on LB-agar plates with 5 μg/ml erythromycin and analyzed by colony PCR for successful deletion of the aprE gene. A single erythromycin-sensitive clone with the correct deleted aprE gene was isolated and designated Bli #002

amyB Gene Deletion Strain Bli #003

Electrocompetent Bacillus licheniformis Bli #002 cells were prepared as described above and transformed with 1 μg of pDel004 amyB gene deletion plasmid isolated from E. coli Ec #098 following plating on LB-agar plates containing 5 μg/ml erythromycin at 30° C.

The gene deletion procedure was performed as described for the aprE gene.

The deletion of the amyB gene was analyzed by PCR with oligonucleotides SEQ ID 09 and SEQ ID 10. The resulting Bacillus licheniformis strain with a deleted aprE and deleted amyB gene is designated Bli #003.

sigF Gene Deletion Strain Bli #004

Electrocompetent Bacillus licheniformis Bli #003 cells were prepared as described above and transformed with 1 μg of pDel005 sigF gene deletion plasmid isolated from E. coli Ec #098 following plating on LB-agar plates containing 5 μg/ml erythromycin at 30° C.

The gene deletion procedure was performed as described for the aprE gene.

The deletion of the sigF gene was analyzed by PCR with oligonucleotides SEQ ID 12 and SEQ ID 13. The resulting Bacillus licheniformis strain with a deleted aprE, a deleted amyB gene and a deleted sigF gene is designated Bli #004. Bacillus licheniformis strain Bli #004 is no longer able to sporulate as described (WO97/03185).

### Poly-Gamma Glutamate Synthesis Genes Deletion Strain Bli #008

Electrocompetent Bacillus licheniformis Bli #004 cells were prepared as described above and transformed with 1 μg of pDel007 pga gene deletion plasmid isolated from E. coli Ec #098 following plating on LB-agar plates containing 5 μg/ml erythromycin at 30° C.

The gene deletion procedure was performed as described for the deletion of the aprE gene.

The deletion of the pga genes was analyzed by PCR with oligonucleotides SEQ ID 15 and SEQ ID 16 The resulting Bacillus licheniformis strain with a deleted aprE, a deleted amyB gene, a deleted sigF gene and a deleted pga gene cluster is designated Bli #008.

### bpr Gene Deletion Strain Bli #74

Electrocompetent Bacillus licheniformis Bli #008 cells were prepared as described above and transformed with 1 μg of pDe1041 gene deletion plasmid isolated from E. coli Ec #098 following plating on LB-agar plates containing 5 μg/ml erythromycin at 30° C.

The gene deletion procedure was performed as described for the aprE gene.

The deletion of the bpr gene was analyzed by PCR with oligonucleotides SEQ ID 23 and SEQ ID 24 The resulting Bacillus licheniformis strain with the additional deleted bpr gene is designated Bli #074.

### vpr Gene Deletion Strain Bli #75

Electrocompetent Bacillus licheniformis Bli #008 cells were prepared as described above and transformed with 1 μg of pDe1040 gene deletion plasmid isolated from E. coli Ec #098 following plating on LB-agar plates containing 5 μg/ml erythromycin at 30° C.

The gene deletion procedure was performed as described for the aprE gene.

The deletion of the vpr gene was analyzed by PCR with oligonucleotides SEQ ID 19 and SEQ ID 20. The resulting Bacillus licheniformis strain with the additional deleted vpr gene is designated Bli #075.

### vpr Gene Deletion Strain Bli #76

Electrocompetent Bacillus licheniformis Bli #074 cells were prepared as described above and transformed with 1 μg of pDe1040 gene deletion plasmid isolated from E. coli Ec #098 following plating on LB-agar plates containing 5 μg/ml erythromycin at 30° C.

The gene deletion procedure was performed as described for the aprE gene.

The deletion of the vpr gene was analyzed by PCR with oligonucleotides SEQ ID 19 and SEQ ID 20. The resulting Bacillus licheniformis strain with the additional deleted vpr gene is designated Bli #076.

ispA Gene Deletion Strains of Bacillus licheniformis

The ispA gene (SEQ ID 25) was partially deleted in various B. licheniformis strains (Table 1) using the ispA gene deletion plasmid pDe1042 and the procedure described before. The correct partial deletion of the ispA gene was analyzed by PCR with oligonucleotides SEQ ID 27 and SEQ ID 28.

TABLE 1

| Parental | Gene | Resulting |
|---|---|---|
| Strain | deletion | strain |
| Bli#008 | ispA | Bli#083 |
| Bli#074 | ispA | Bli#084 |
| Bli#075 | ispA | Bli#085 |
| Bli#076 | ispA | Bli#086 |

### Plasmids

pEC194RS—Bacillus Temperature Sensitive Deletion Plasmid.

The plasmid pE194 (Villafane, et al (1987): J. Bacteriol. 169(10), 4822-4829) is PCR-amplified with oligonucleotides SEQ ID 01 and SEQ ID 02 with flanking PvuII sites, digested with restriction endonuclease PvuII and ligated into vector pCE1 digested with restriction enzyme SmaI. pCE1 is a pUC18 derivative, where the BsaI site within the ampicillin resistance gene has been removed by a silent mutation. The ligation mixture was transformed into E. coli DH10B cells (Life technologies).

Transformants were spread and incubated overnight at 37° C. on LB-agar plates containing 100 μg/ml ampicillin. Plasmid DNA was isolated from individual clones and analyzed for correctness by restriction digest. The resulting plasmid is named pEC194S.

The type-II-assembly mRFP cassette is PCR-amplified from plasmid pBSd141R (accession number: KY995200) (Radeck, J., Mascher, T. 2017; Sci. Rep. 7: 14134) with oligonucleotides SEQ ID 03 and SEQ ID 04, comprising additional nucleotides for the restriction site BamHI. The PCR fragment and pEC194S were restricted with restriction enzyme BamHI following ligation and transformation into E. coli DH10B cells (Life technologies). Transformants were spread and incubated overnight at 37° C. on LB-agar plates containing 100 μg/ml ampicillin. Plasmid DNA was isolated from individual clones and analyzed for correctness by restriction digest. The resulting plasmid pEC194RS carries the mRFP cassette with the open reading frame opposite to the reading frame of the erythromycin resistance gene.

pDel003—aprE Gene Deletion Plasmid

The gene deletion plasmid for the aprE gene of Bacillus licheniformis was constructed with plasmid pEC194RS and the gene synthesis construct SEQ ID 05 comprising the genomic regions 5′ and 3′ of the aprE gene flanked by BsaI sites compatible to pEC194RS. The type-II-assembly with restriction endonuclease BsaI was performed as described (Radeck et al., 2017) and the reaction mixture subsequently transformed into E. coli DH10B cells (Life technologies). Transformants were spread and incubated overnight at 37° C. on LB-agar plates containing 100 μg/ml ampicillin. Plasmid DNA was isolated from individual clones and analyzed for correctness by restriction digest. The resulting aprE deletion plasmid is named pDel003.

pDel004—amyB Gene Deletion Plasmid

The gene deletion plasmid for the amyB gene of Bacillus licheniformis was constructed as described for pDel003, however the gene synthesis construct SEQ ID 08 comprising the genomic regions 5′ and 3′ of the amyB gene flanked by BsaI sites compatible to pEC194RS was used. The resulting amyB deletion plasmid is named pDel004.

pDel005—sigF Gene Deletion Plasmid

The gene deletion plasmid for the sigF gene (spoIIAC gene) of Bacillus licheniformis was constructed as described for pDel003, however the gene synthesis construct SEQ ID 11 comprising the genomic regions 5′ and 3′ of the sigF gene flanked by BsaI sites compatible to pEC194RS was used. The resulting sigF deletion plasmid is named pDel005.

pDel007—Poly-Gamma-Glutamate Synthesis Genes Deletion Plasmid

The deletion plasmid for deletion of the genes involved in poly-gamma-glutamate (pga) production, namely ywsC (pgsB), ywtA (pgsC), ywtB (pgsA), ywtC (pgsE) of Bacillus licheniformis was constructed as described for pDel003, however the gene synthesis construct SEQ ID 14 comprising the genomic regions 5′ and 3′ flanking the ywsC, yvwtA (pgsC), yvwtB (pgsA), yvwtC (pgsE) genes flanked by BsaI sites compatible to pEC194RS was used. The resulting pga deletion plasmid is named pDel007.

pDel040—vpr Deletion Plasmid

The gene deletion plasmid for the vpr gene (SEQ ID 17) of Bacillus licheniformis was constructed as described for pDel003, however the gene synthesis construct SEQ ID 18 comprising the genomic regions 5′ and 3′ of the vpr gene flanked by BsaI sites compatible to pEC194RS was used. The resulting vpr deletion plasmid is named pDe1040.

pDel041—bpr Deletion Plasmid

The gene deletion plasmid for the bpr gene (SEQ ID 21) of Bacillus licheniformis encoding the Bpr bacillopeptidase was constructed as described for pDel003, however the gene synthesis construct SEQ ID 22 comprising the genomic regions 5′ and 3′ of the bpr gene flanked by BsaI sites compatible to pEC194RS was used. The resulting bpr deletion plasmid is named pDe1041.

pDel042—ispA Deletion Plasmid

The gene deletion plasmid for the ispA gene (SEQ ID 25) of Bacillus licheniformis encoding the intracellular protease IspA was constructed as described for pDel003, however the gene synthesis construct SEQ ID 26 comprising the genomic region 5′ of the ispA and the 3′ end of the ispA gene flanked by BsaI sites compatible to pEC194RS was used. The resulting ispA (partial) deletion plasmid is named pDe1042.

pBW424—Low Copy Bacillus T2A—E. coli Shuttle Vector

The plasmid pBW424 is a low-copy Bacillus-E. coli shuttle vector based on the pBS72 origin of replication (Titok, M. A. et al. 2003, Plasmid, 49(1), 53-62.) with the kanamycin resistance gene of pUB110, a ColE1 origin of replication for E. coli and a type-II-assembly cassette for subsequent cloning of expression construct. The plasmid was constructed as described below with gene synthesis constructs optimized for low numbers of restriction endonuclease sites and flanked by restriction endonuclease BsaI sites.

SEQ ID 29 comprises the origin of replication of pBS72 (accession number: AY102630) which has been optimized for less endonuclease restriction sites compared to the published sequence.

SEQ ID 30 comprises the modified mRFP cassette from plasmid pBSd141R (accession number: KY995200; Radeck et al., 2017; Sci. Rep. 7: 14134) with flanking type-II restriction enzyme sites of BpiI, the terminator region of the aprE gene from Bacillus licheniformis.

SEQ ID 31 comprises a modified kanamycin resistance gene of pUB110 and a modified ColE1 origin of replication. The plasmid pBW424 was assembled by type-II restriction cloning described by Radeck et al. 2017 using BsaI restriction endonucelase. The ligation mixture was transformed into E. coli DH10B cells (Life technologies). Transformants were spread and incubated overnight at 37° C. on LB-agar plates containing 20 μg/ml kanamycin. Plasmid DNA was isolated from individual clones and analyzed for correctness by restriction digest following the sequencing. The resulting plasmid is named pBW424.

pBW1327—GFP Expression Plasmid pBW1327-GFPmut2

A constitutive sigA promoter (nt 13-90 of SEQ ID 32) was used for expression of the GFP gene and ordered as gene synthesis fragment. The GFPmut2 gene variant (accession number AF302837) with flanking BpiI restriction sites (SEQ ID 40) was ordered as gene synthesis fragment. The gene expression construct comprising the promoter and the GFPmut2 variant was cloned into plasmid pBW424 by type-II-assembly with restriction endonuclease BpiI as described (Radeck et al., 2017; Sci. Rep. 7: 14134) and the reaction mixture subsequently transformed into electrocompetent E. coli Ec #098 cells. Transformants were spread and incubated overnight at 37° C. on LB-agar plates containing 20 μg/ml kanamycin. Plasmid DNA was isolated from individual clones and analyzed for correctness by restriction digest and sequencing. The resulting plasmid is named pBW1327-GFPmut2.

Plasmid pUK57S: Type-II-assembly destination shuttle plasmid

The BsaI site within the repU gene as well as the BpiI site 5′ of the kanamycin resistance gene of the protease expression plasmid pUK56S (WO2019016051) were removed in two sequential rounds by applying the Quickchange mutagenesis Kit (Agilent) with quick change oligonucleotides SEQ ID 33, SEQ ID 34, SEQ ID 35, SEQ ID 36 respectively.

Subsequently the plasmid was restricted with restriction endonuclease NdeI and SacI following ligation with a modified type-II assembly mRFP cassette, cut with enzymes NdeI and SacI.

The modified mRFP cassette (SEQ ID 37) comprises the mRPF cassette from plasmid pBSd141 R (accession number: KY995200)(Radeck et al., 2017) with flanking type-II restriction enzyme sites of BpiI, the terminator region of the aprE gene from Bacillus licheniformis and flanking NdeI and SacI sites and was ordered as gene synthesis fragment.

The ligation mixture was transformed into E. coli DH10B cells (Life technologies). Transformants were spread and incubated overnight at 37° C. on LB-agar plates containing 100 μg/ml ampicillin. Plasmid DNA was isolated from individual clones and analyzed for correctness by restriction digest. The resulting plasmid is named pUK57S.

Plasmid pUK57: Type-II-Assembly Destination Bacillus Plasmid

The backbone of pUK57S was PCR-amplified with oligonucleotides SEQ ID 38 and SEQ ID 39 comprising additional EcoRI sites. After EcoRI and DpnI restriction the PCR fragment was ligated using T4 ligase (NEB) following transformation into B. subtilis Bs #056 cells made competent according to the method of Spizizen (Anagnostopoulos, C. and Spizizen, J. (1961). J. Bacteriol. 81, 741-746) following plating on LB-agar plates with 20 μg/ml Kanamycin. Correct clones of final plasmid pUK57 were analyzed by restriction enzyme digest and sequencing.

### ß-Galactosidase Expression Plasmids

The ß-Galactosidase expression plasmids pUK57-Lac36 and pUK57-Lac14 are each composed of 3 parts—the plasmid backbone of pUK57, the promoter of the aprE gene from Bacillus licheniformis from pCB56C (U.S. Pat. No. 5,352,604) and the codon-optimized ß-Galactosidase genes SEQ ID 44 (ß-Galactosidase protein accession number WP_033513636, SEQ ID 43) and SEQ ID 46 (ß-Galactosidase protein accession number WP_055309314, SEQ ID 45) respectively.

The promoter fragment is PCR-amplified with oligonucleotides SEQ ID 41 and SEQ ID 42 comprising additional nucleotides for the restriction endonuclease BpiI. The type-II-assembly with restriction endonuclease BpiI was performed as described (Radeck et al., 2017) and the reaction mixture subsequently transformed into B. subtilis Bs #056 cells made competent according to the method of Spizizen (Anagnostopoulos, C. and Spizizen, J. (1961). J. Bacteriol. 81, 741-746) following plating on LB-agar plates with 20 μg/ml Kanamycin). Correct clones of final plasmids pUK57-Lac36 and pUK57-Lac14 were analyzed by restriction enzyme digest and sequencing.

### Example 1: Release of Intracellularly Accumulated 13-Galactosidase into in the Cultivation Supernatant by Cell Lysis

Bacillus licheniformis strain expressing and accumulating intracellularly ß-Galactosidase was cultivated in a fed-batch process in 21 L stirred tank reactor at temperature- and pH-controlled conditions and glucose and air supply as described (WO 2020/169563 A1). At the end of the fermentation process the process of cell lysis was monitored by measuring the ß-Galactosidase activity in the supernatant.

The activity of ß-Galactosidase was measured by the hydrolysis of an artificial Lactase substrate (2-Nitrophenyl p-D-galactopyranoside; OPNG) whereby 2-Nitrophenol (2-NP) is released with consequently a change of the extinction coefficient at 405 nm over time. The assay was performed in 50 mM MES (pH6.5), 1 mM MgSO4, 0.45 g/L Brij L23 as assay buffer for dilution of the sample and the substrate (final substrate concentration of OPNG is 1.46 mM). The reaction time was monitored for 10 min at 405 nm using at suitable dilution. For calibration either 2-Nitrophenol was used or a suitable reference beta-galactosidase.

To analyze the process of cell lysis was conducted as described in the following.

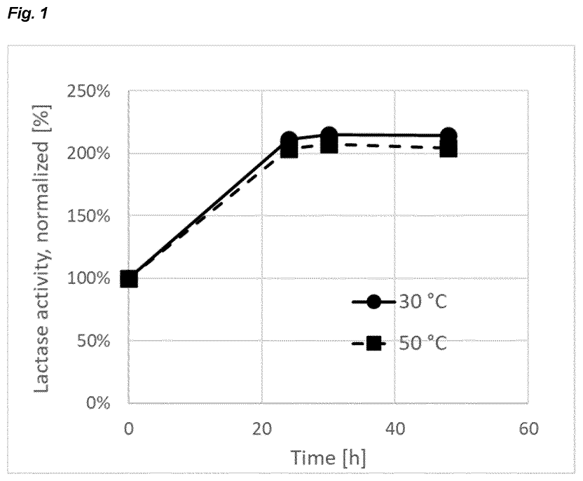

After the end of fermentation, the stirred speed was reduced, and the glucose feed and air gassing switched off. 20 mL of fermentation broth was transferred into 50 mL Falcon tubes each. The pH of the fermentation broth was corrected to pH=7.0 by addition of NH₄OH (20 wt-%) or H₂SO₄ (20 wt-%). The incubation of the fermentation broth was conducted in two thermomixers (Eppendorf) at 200 rpm shaking rate, one set to 30° C., the other set to 50° C. Samples were taken at 24, 30 and 48 h. Each sample was sterile filtered with 0.2 μm syringe filters and the ß-Galactosidase activity measured. The ß-Galactosidase activity values for each temperature were normalized to the values at t=0 h. As seen in FIG. 1, surprisingly the ß-Galactosidase activity in the supernatant increased by more than 100% for both temperatures within the first 24 h levelling off on a plateau upon further incubation. The intracellularly accumulated ß-Galactosidase was released into the supernatant by lysis within the first 24 h, explaining the increase in supernatant activity by more than 100%. Even incubation at ambient temperature (30° C.) resulted in cell lysis making the ß-Galactosidase available for purification.

### Example 2: Induction of Cell Lysis by Incubation

The fermentation process for the intracellular accumulation of ß-Galactosidase was conducted as described in Example 1. At the end of fermentation 500 g broth each was then transferred into a pH and temperature controlled small-scale stirred tank reactor system (2 L) for parallel incubation. The incubation conditions were set to a stirrer rate of 500 rpm, temperature control at values of 30° C., 40° C. and 45° C. respectively, pH control at 7.0 and automated antifoam addition when foam formation was detected. The three batches were regularly sampled and directly analyzed for optical density at 600 nm. The optical density measurement was performed with a standard photometer at wavelength of 600 nm and the usage of standard polystyrene cuvettes with standard width of 1 cm. The measured optical density (alternatively: extinction) is defined as the decimal logarithm of the ratio of light intensity before sample passage to the light intensity after sample passage. Consequently, the measured values do not possess a physical unit, but are given in arbitrary units “AU”.

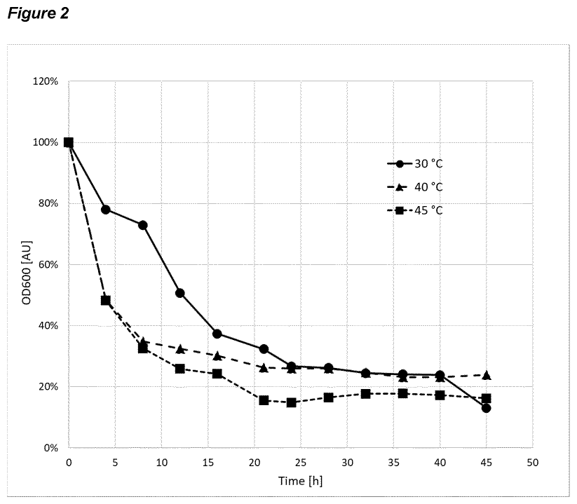

The relative optical density, with the start timepoint (t=Oh) being set to 100%, is plotted for each temperature against the incubation time as shown in in FIG. 2. The optical density was highest directly at the end of fermentation representing intact cells. Incubation without aeration and feed of nutrients (in particular the C-source) results in cell lysis as observed with a sharp drop of optical density over the time course of 45 h.

### Example 3: Analysis of the Amount of the Cytosolic 13-Galactosidase in the Cultivation Supernatant

Bacillus licheniformis strains with genetic modifications as listed in Table 2 were cultivated in six replicates each in a microtiter plate-based fed-batch process (Habicher et al., 2019 Biotechnol J.; 15(2), 1900088).

TABLE 2
B. licheniformis mutant strains

| Strain | Genotype |
|---|---|
| Bli#008 | B. licheniformis DaprE DamyB DsigF Dpga |
| Bli#074 | B. licheniformis DaprE DamyB DsigF Dpga Dbpr |
| Bli#075 | B. licheniformis DaprE DamyB DsigF Dpga Dvpr |
| Bli#076 | B. licheniformis DaprE DamyB DsigF Dpga Dbpr Dvpr |
| Bli#083 | B. licheniformis DaprE DamyB DsigF Dpga DispA |
| Bli#084 | B. licheniformis DaprE DamyB DsigF Dpga Dbpr DispA |
| Bli#085 | B. licheniformis DaprE DamyB DsigF Dpga Dvpr DispA |
| Bli#086 | B. licheniformis DaprE DamyB DsigF Dpga Dbpr Dvpr DispA |

All cultivations were conducted in an orbital shaker with a diameter of 25 mm (Innova 42, New Brunswick Scientific, Eppendorf AG; Hamburg, Germany) at 30° C. and 400 rpm. Strains were cultivated in two subsequent precultures in FlowerPlates (MTP-48-OFF, m2p-labs GmbH) for synchronization of growth. The first preculture was carried out in 800 μl TB medium inoculated with a fresh single colony from the strain streaked onto LB agar plates. After 20 h, the second preculture containing 800 μl V3 minimal medium (Meissner et al., 2015, Journal of industrial microbiology & biotechnology 42 (9): 1203-1215) was inoculated with 8 μl of the first preculture and cultivated for 24 h. Microtiter plate-based fed-batch main cultivations were conducted using 48-well round- and deep-well-microtiter plates with glucose-containing polymer on the bottom of each well (FeedPlate, article number: SMFP08004, Kuhner Shaker GmbH; Herzogenrath, Germany). 70 μl of the second preculture were used to inoculate 700 μl V3 minimal medium without glucose. Main cultures were incubated for 72 h. Precultures were covered with a sterile gas-permeable sealing foil (AeraSeal film, Sigma-Aldrich) to avoid contamination. FeedPlates were sealed with a sterile gas-permeable, evaporation reducing foil (F-GPR48-10, m2p-labs GmbH) to reduce evaporation and to avoid contamination.

At the end of the fermentation process, 250 microliter samples were withdrawn and the supernatant of each cultivation recovered by centrifugation (13.000 g 10 min, 4° C.) and filtration (0.2 micrometer).

The activity of the B. licheniformis endogenous ß-Galactosidase was measured by the hydrolysis of an artificial Lactase substrate (2-Nitrophenyl p-D-galactopyranoside; OPNG) whereby 2-Nitrophenol (2-NP) is released with consequently a change of the extinction coefficient at 405 nm over time. The assay was performed in 50 mM MES (pH6.5), 1 mM MgSO4, 0.45 g/L Brij L23 as assay buffer for dilution of the sample and the substrate (final substrate concentration of OPNG is 1.46 mM). The reaction time was monitored for 10 min at 405 nm using at suitable dilution. For calibration either 2-Nitrophenol was used or a suitable reference beta-galactosidase.

The completion of cell lysis was tested by measuring the beta-galactosidase activity of the supernatant of samples being either untreated with lysozyme or lysozyme treated (Lysozyme treatment: 60 min incubation at 30° C. with 10 g/L Lysozyme and 25 U/mL Benzonase). If the beta-galactosidase activity was identical for the samples with and without lysozyme the lysis was complete already without lysozyme.

The relative increased amount of ß-Galactosidase released through ispA gene-inactivation-mediated release was determined by setting the ß-Galactosidase activity of the corresponding ispA(+) strain to 100%.

Table 3 summarizes the relative increase of ß-Galactosidase in Bacillus licheniformis strains with inactivated ispA gene. Surprisingly the inactivation of the ispA gene leads to increased release of intracellular proteins as determined for the ß-Galactosidase and eases subsequent recovery the ß-Galactosidase in downstream processing.

TABLE 3

| | | rel. | |
|---|---|---|---|
| | | β-Galactosidase | |
| | | Activity | |
| Strain | Genotype | [%] | % CV |
| Bli#008 | Bli#008 | 100 | 39 |
| Bli#083 | Bli#008 DispA | 514 | 38 |
| Bli#074 | Bli#008 Dbpr | 100 | 11 |
| Bli#084 | Bli#008 Dbpr DispA | 487 | 115 |
| Bli#075 | Bli#008 Dvpr | 100 | 12 |
| Bli#085 | Bli#008 Dvpr DispA | 349 | 33 |
| Bli#076 | Bli#008 Dbpr Dvpr | 100 | 25 |
| Bli#086 | Bli#008 Dbpr Dvpr DispA | 803 | 104 |

### Example 4: Increased Release of Heterologous Intracellular ß-Galactosidase in Bacillus Licheniformis Strain with Inactivated ispA Gene

Bacillus licheniformis strains as listed in Table 2 were made competent as described above. ß-Galactosidase expression plasmids pUK57-Lac36 and pUK57-Lac14 were isolated from Bs #056 Bacillus cells. Plasmids pUK57-Lac36 and pUK57-Lac14 were transformed in the indicated strains and plated on LB-agar plates with 20 μg/μl kanamycin. Individual clones were analyzed for correctness of the plasmid DNA by restriction digest and Sanger sequencing. The resulting B. licheniformis expression strains are listed in Table 4.

TABLE 4
Overview on B. licheniformis expression strains

| B. licheniformis | Expression | B. licheniformis |
|---|---|---|
| Expression strain | plasmid | strain |
| BES#173 | pUK57-Lac36 | Bli#076 |
| BES#174 | pUK57-Lac36 | Bli#086 |
| BES#175 | pUK57-Lac14 | Bli#076 |
| BES#176 | pUK57-Lac14 | Bli#086 |

Bacillus licheniformis expression strains BES #173, BES #174, BES #175 and BES #176 were cultivated in two independent experiments with six replicates each as described in example 1.

At the end of the fermentation process, 250 microliter whole broth samples were withdrawn and the supernatant of each cultivation recovered by centrifugation (13.000 g 10 min, 4° C.) and filtration (0.2 micrometer). The remaining cell pellet was resuspended in an equal volume of resuspension buffer (0.9% NaCl solution containing lysozyme at 10 mg/ml and 25 U Benzonase (Merck) following incubation at 30° C. for 60 min.

The activity of the heterologous ß-Galactosidase enzymes was measured as described in Example 1.

The relative increased amount of ß-Galactosidase released through ispA gene-inactivation-mediated release was determined as described in the following:

The cell-lysis mediated release of lactase was calculated at the percentage of lactase activity of the supernatant sample divided by the total lactase activity, that is of both supernatant and lysed cells.

Table 5 summarizes the cell-lysis mediated release of ß-Galactosidase in Bacillus licheniformis strains with and without inactivated ispA gene at the end of the cultivation process. Surprisingly the inactivation of the ispA gene leads to increased release of intracellular expressed heterologous ß-Galactosidase proteins.

TABLE 5

| | | | | rel. | |
|---|---|---|---|---|---|
| Bacillus | | | | β-Galactosidase | |
| Expression | | | | Activity | |
| Strain | Strain | Plasmid | Genotype | [%] | % CV |
| BES#173 | Bli#076 | pUK57- | Bli#008 Dbpr | 54 | 3 |
| | | Lac36 | Dvpr | | |
| BES#174 | Bli#086 | pUK57- | Bli#008 Dbpr | 67 | 4 |
| | | Lac36 | Dvpr DispA | | |
| BES#175 | Bli#076 | pUK57- | Bli#008 Dbpr | 42 | 3 |
| | | Lac14 | Dvpr | | |
| BES#176 | Bli#086 | pUK57- | Bli#008 Dbpr | 59 | 6 |
| | | Lac14 | Dvpr DispA | | |

### Example 5: Release of Intracellularly Accumulated 13-Galactosidase into in the Cultivation Supernatant by Cell Lysis

Bacillus licheniformis strains BES #175 and BES #176 (Table 4) expressing and accumulating intracellularly ß-Galactosidase Lac-14 were cultivated in a fed-batch process in 21 L stirred tank reactors at temperature- and pH-controlled conditions and glucose and air supply as described (WO 2020/169563 A1). At the end of the fermentation process the process of cell lysis was monitored by measuring the ß-Galactosidase activity in the supernatant as described in Example 1.

The process of cell lysis was monitored under different conditions as described in the following.

After the end of the fermentations the stirred speed was reduced and the glucose feed and air gassing switched off. 800 ml of fermentation broth was transferred into two 2 L stirred tank reactors for each strain and the pH of the fermentation broths were corrected to pH=7.0 by addition of NH₄OH (20 wt-%) or H₂SO₄ (20 wt-%). The incubation of the fermentation broths of BES #175 and BES #176 were set to a stirrer speed rate of 500 rpm, and for each strain one vessel was set to 30° C., and the other vessel set to 50° C. Whole-broth samples were taken at 0 h, 0.5 h, 4 h, and 4 h intervals up to 48 h and the supernatant of each cultivation recovered by centrifugation (13.000 g 10 min, 4° C.) and filtration (0.2 micrometer). The remaining cell pellet was resuspended in an equal volume of resuspension buffer (0.9% NaCl solution containing lysozyme at 10 mg/ml and 25 U Benzonase (Merck) following incubation at 30° C. for 60 min. The activity of the heterologous ß-Galactosidase enzymes was measured as described in Example 1. The cell-lysis mediated release of lactase was calculated as the percentage of lactase activity of the supernatant sample divided by the total lactase activity, that is of both supernatant and lysed cells.

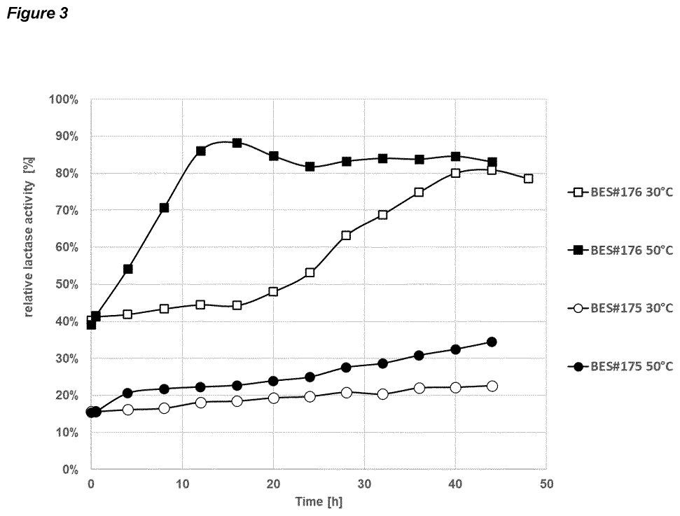

As seen in FIG. 3, surprisingly the ß-Galactosidase activity in the supernatant of the ispA knockout strain BES #176 at the end of the fermentation (t=0 h) is higher (40%) compared to the amount of ß-Galactosidase activity in the supernatant of BES #175 strain (15%) with intact ispA gene relative to the total ß-Galactosidase produced in each strain which is set to 100%. Prolonged incubation time and temperature leads to further cell lysis and release of increased intracellularly accumulated ß-Galactosidase. Again, the ispA knockout strain BES #176 shows cell lysis up to more than 80% for both temperatures 30° C. and 50° C. at 40 hours and 10 hours respectively, levelling off on a plateau upon further incubation. In contrast, cell lysis and hence release of the intracellularly accumulated ß-Galactosidase was much lower for BES #175 (intact ispA gene) for both temperatures 30° C. and 50° C. and did not reach the levels of BES #176.

1. A method for producing at least one polypeptide of interest in a Bacillus host cell, said method comprising the steps of
a) providing a Bacillus host cell comprising a polynucleotide encoding the at least one polypeptide of interest, wherein the Bacillus host cell is a ispA knock-out cell,
b) cultivating the Bacillus host cell under conditions which allow for intracellular accumulation of said at least one polypeptide of interest, thereby producing said at least one polypeptide of interest,
c) stop of aeration and addition of feed, and
d) incubating the Bacillus host cell for at least five hours.

2. The method of claim 1, wherein the at least one polypeptide of interest does not comprise a functional secretion sequence.

3. The method of claim 1, wherein the host cell in step d) is incubated at an increased temperature.

4. The method of claim 1, wherein the cells are incubated in step d) for 5 to 40 hours.

5. The method of claim 1, wherein at least 70% of cells are lysed after d).

6. The method of claim 1, wherein one or more of the following genes have been knocked-out: one or more extracellular proteases, the intracellular protease aprX, an amylase, an amyloglucosidase, a cellulase, a chitinase, a lipase, a phospholipase, an esterase, an invertase, an arylsulfatase, a mannanase, a pectinase and/or a pectate lyase.

7. The method of claim 6, wherein the one or more extracellular proteases is/are aprE, mpr, bpr, vpr, epr, nprE, nprB and/or wprA,

8. The method of claim 1, wherein the at least one polypeptide of interest is an enzyme.

9. The method of claim 1, wherein the at least one polypeptide of interest is selected from the group consisting of a ß-galactosidase, xylanase, protease, phytase, casein, lactoferrin, dehydrogenase, nitrilase, transaminase, laccase, lipase, esterase, lactase, mannanase and lactoferrin.

10. The method of claim 1, wherein the Bacillus host cell is a Bacillus alcalophilus, Bacillus amyloliquefaciens, Bacillus brevis, Bacillus cereus, Bacillus circulans, Bacillus clausii, Bacillus coagulans, Bacillus firmus, Bacillus globigii, Bacillus halodurans, Bacillus lautus, Bacillus lentus, Bacillus licheniformis, Bacillus paralicheniformis, Bacillus megaterium, Bacillus methanolicus, Bacillus methylotrophicus, Bacillus mojavensis, Bacillus pumilus, Geobacillus stearothermophilus (Bacillus stearothermophilus), Bacillus thuringiensis or Bacillus velezensis host cell.

11. The method of claim 1, wherein a fermentation broth comprising lysed cells is obtained in step d), and wherein the method further comprises a step of isolating the at least one polypeptide of interest from the lysed fermentation broth.

12. A Bacillus host cell comprising a polynucleotide encoding at least one polypeptide of interest lacking a functional secretion sequence, wherein the ispA gene has been inactivated in the Bacillus host cell.

13. The Bacillus host cell of claim 12, wherein the at least one polypeptide of interest is an enzyme.

14. A method of producing a Bacillus host cell comprising
a) providing a Bacillus cell comprising an ispA gene,
b) inactivating the ispA gene, and
c) introducing at least one polynucleotide encoding at least one polypeptide of interest into the cell, wherein the at least one polypeptide of interest lacks a functional secretion sequence.

15. The method of claim 14, wherein the polypeptide of interest is an enzyme.
